SEQUENCE LISTING (213 sequences)

SEQ ID NO: 1 - DNA, 1947 bp, Homo sapiens
  CDS (35)..(1564)
gtggtttctaacgctgaacttaaaaagtgttgagatgaataatgaagaggaccttctgcaggaggactcaacaagagatgaaggcaatgagactgaagccaacagcatgaacacattaagaaggacaaggaagaaagtcactaaaccatatgtttgttcaactgaagtgggagaaacggatatgtccaattcaaatgattgcatgagggacagcagtcaaattttgaccccacctcaactctcttctagaatgaaacacattagacaagccatggccaaaaatcgcctccagtttgtacgatttgaagcaacagacctccacggcgtgtccaggtctaagactatccctgcacacttttttcaagagaaagtgagccatggtgtttgcatgccccgaggttatcttgaagtgataccaaatccaaaggacaatgaaatgaataacataagagccacatgttttaatagcgacatagtcctaatgccagagttatcaacctttagagttttgccatgggctgacagaactgcaagagtgatatgtgataccttcactgtgactggtgagcctcttttgacttccccaaggtacattgcaaagaggcagctgagccatctgcaggcctctggcttttccctgctttctgctttcatctatgatttttgcatttttggtgtgcccgaaattttaaattcaaagattatatcttttcctgctttaacatttttaaataaccatgatcagcccttcatgcaggaacttgttgatggcttgtatcacactggagccaatgtcgagagtttttcctcctctaccaggcctggtcagatggaaatctctttcctgcctgaatttggcattagctcagctgataatgcatttaccctcagaacaggtgtcaaagaagtggcaaggaaatataattacattgccagcttcttcattgagactggattttgtgattcagggattttgtctcatagtctctgggatgtcgataggaagaaaaacatgttctgcagcacttctggaactgagcagctcacgatcactgggaaaaaatggttggcaggactcttgaagcactctgctgcgctcagctgcctgatggcgccttctgttagctgccgaaagcgttattccaaggacaggaaagacctgaagaagagtgtgcctacaacatggggatacaatgacaacagctgtatatttaatatcaaatgtcatggagagaaaggcacccggatagaaaataaactaggctcagcaacagcaaacccttacttggtgctggctgcaactgttgctgccggcttagatggacttcatagcagtaatgaggtcttggctggtccagatgagagcacagacttttaccaagtggaaccttctgagatccctttaaaactagaagatgcccttgtggcactggaagaagatcaatgccagagacaggctctaggagaaacctttattcgatattttgttgccatgaagaaatatgagttggagaatgaagaaatagctgcagagagaaataaattcttagagtattttatttagaatagagctcacaactactcctttagacatgtaattgttacttaaagctaatctaccaaaacaaaaagactgaacttttgtaattaacaacagcaacaataacaagattacgaatgcttttgacttttttttttgtccatggaatatttgacagatgaagtaggactgctgagaagattctgataacagaaacaaacaacaaagttgtggtgaagctataatatgcaaacttaccagatcttgtcagtcatttcctatgtgtatgttgacctggataagaatatccaattttgggtggcaataaatatattcgcacattaagaaaaaaaagacttaagcattagaaagcaaaatttaaatgactaaaaaaaaaaaaaaaaaaa

SEQ ID NO: 2 - protein, 509 aa, Homo sapiens
MNNEEDLLQEDSTRDEGNETEANSMNTLRRTRKKVTKPYVCSTEVGETDMSNSNDCMRDSSQILTPPQLSSRMKHIRQAMAKNRLQFVRFEATDLHGVSRSKTIPAHFFQEKVSHGVCMPRGYLEVIPNPKDNEMNNIRATCFNSDIVLMPELSTFRVLPWADRTARVICDTFTVTGEPLLTSPRYIAKRQLSHLQASGFSLLSAFIYDFCIFGVPEILNSKIISFPALTFLNNHDQPFMQELVDGLYHTGANVESFSSSTRPGQMEISFLPEFGISSADNAFTLRTGVKEVARKYNYIASFFIETGFCDSGILSHSLWDVDRKKNMFCSTSGTEQLTITGKKWLAGLLKHSAALSCLMAPSVSCRKRYSKDRKDLKKSVPTTWGYNDNSCIFNIKCHGEKGTRIENKLGSATANPYLVLAATVAAGLDGLHSSNEVLAGPDESTDFYQVEPSEIPLKLEDALVALEEDQCQRQALGETFIRYFVAMKKYELENEEIAAERNKFLEYFI

SEQ ID NO: 3 - DNA, 2946 bp, Homo sapiens
  CDS (346)..(1650)
ggaaagccggctcaccttcgcctccccctgcggctgggaggagaggaaatatcccatggctgactgtgccaaggaggtgtctgagccagccctcccggcccgagggcagggcaggtggccctgagagataagccaatcccgcagctgcagatgaggagttctgagaagcattgctcaggacagcggtaaatcacttcttggaggtgccctgcacgccggtcctgggagcaggcggcctcccgggggtgcgggagccccactcctccgtggtgtgttccatttgcttcccacatctggaggagctgacgtgccagcctcccccagcaccacccagggacgggaggcatgagccggtcaaggcacctgggcaaaatccggaagcgtctggaagatgtcaagagccagtgggtccggccagccagggctgactttagtgacaacgagagtgcccggctggccacggacgccctcttggatgggggttctgaagcctactggcgggtgctcagccaggaaggcgaggtggacttcttgtcctcggtggaggcccagtacatccaggcccaggccagggagcccccgtgtcccccagacaccctgggaggggcggaagcaggccctaagggactggactccagctccctacagtccggcacctacttccctgtggcctcagagggcagcgagccggccctactgcacagctgggcctcagctgagaagccctacctgaaggaaaaatccagcgccactgtgtacttccagaccgtcaagcacaacaacatcagagacctcgtccgccgctgcatcacccggactagccaggtcctggtcatcctgatggatgtgttcacggatgtggagatcttctgtgacattctagaggcagccaacaagcgtggggtgttcgtttgtgtgctcctggaccagggaggtgtgaagctcttccaggagatgtgtgacaaagtccagatctctgacagtcacctcaagaacatttccatccggagtgtggaaggagagatatactgtgccaagtcaggcaggaaattcgctggccaaatccgggagaagttcatcatctcggactggagatttgtcctgtctggatcttacagcttcacctggctctgcggacacgtgcaccggaacatcctctccaagttcacaggccaggcggtggagctgtttgacgaggagttccgccacctctacgcctcctccaagcctgtgatgggcctgaagtccccgcggctggtcgcccccgtcccgcccggagcagccccggccaatggccgccttagcagcagcagtggctccgccagtgaccgcacgtcctccaaccccttcagcggccgctcggcaggcagccaccccggtacccgaagtgtgtccgcgtcttcagggccctgtagccccgcggccccacacccgcctccaccgccccggttccagccccaccaaggcccttggggagccccgagtccccaggcccacctctccccgcggccccacgacggcccgcccgccgctgtctacagcaacctgggggcctacaggcccacgcggctgcagctggagcagctgggcctggtgccgaggctgactccaacctggaggcccttcctgcaggcctcccctcacttctgaaggtcccatcccctgctgccctccgcaggcccagggctgggcactccctgagacccaaagacccacctcaacgacgagtggcgttgagccacttccctttgaaaagacactcaaaatcactgccatggttcaatgttcccaggccccaggccatccacttgccggcccccaccagttcttgggttccccgctctagtttgacctgtgcagcacattccagaaggttccagggaggttgtggggcagctagaggacaaaatcatgaaaacagagtccctgtcttccagagatcatccggggctttaatattaatggcccccaaaactccgtaagaagcaggaaatgcagcccaagttttacaaatgggtaaacagaggcactgagagatagatggtagtttggtacttctggttcccagtgcccaggaatggtccactcccaagaaattcaggaaagaaagactgaggagaaggtgtgggaacattctggatgtttcgggagagttggggaaactcctcctcttaggaaaggctaatactagggtatccttgggcccaatgaattaggggtgaggccccagaacccgttatctatgagttgtatgggggagccatctgaagctgtagccaccagggatgcagctagctgaggagtttggggtgttgggttggacaaggcaggttagtagactcagattcttgcttcaaagagccttgggctggcctggaggtccctggagtctagactggacctaggagcttgagttgtcaggggccaggactggccccactgcagtgcccaggccagtcttgagcagcagggagggctcagctgtccccagatccaggtgcctctgaccagcctggtcacctcctgaggaataaatgctgaacctcacaagccccatcattcatttcttctcaattcacagtgcccctctttgtttctggggtggaactaggtcctgagggcacagcctagctgagtgcaaagaaatataggatgcttagaaagcatacaggaggggccaggcgtggtggctcatgcctgtaatcccagaactttgggatgccaaggtggttggattacctgagatcaggtggattacctggtctcgagaccagcctgaccaatatggtgaaaccccgtctctactaaaaatacaaaaattaggctgagacaggagaattgcttgaacccaggaagcagaggttgcaatgagctgagattgcatcactgcactccagcatgggcaacaaagcaagactccgtcacagaaaaaaaaaaaaaaaaaaa

SEQ ID NO: 4 - protein, 434 aa, Homo sapiens
MSRSRHLGKIRKRLEDVKSQWVRPARADFSDNESARLATDALLDGGSEAYWRVLSQEGEVDFLSSVEAQYIQAQAREPPCPPDTLGGAEAGPKGLDSSSLQSGTYFPVASEGSEPALLHSWASAEKPYLKEKSSATVYFQTVKHNNIRDLVRRCITRTSQVLVILMDVFTDVEIFCDILEAANKRGVFVCVLLDQGGVKLFQEMCDKVQISDSHLKNISIRSVEGEIYCAKSGRKFAGQIREKFIISDWRFVLSGSYSFTWLCGHVHRNILSKFTGQAVELFDEEFRHLYASSKPVMGLKSPRLVAPVPPGAAPANGRLSSSSGSASDRTSSNPFSGRSAGSHPGTRSVSASSGPCSPAAPHPPPPPRFQPHQGPWGAPSPQAHLSPRPHDGPPAAVYSNLGAYRPTRLQLEQLGLVPRLTPTWRPFLQASPHF

SEQ ID NO: 5 - DNA, 5168 bp, Homo sapiens
  CDS (790)..(2823)
ctggagtctcccctggtcgggagcctcagccttctggagactgacaccaccctcttactcagagacgaatcgttttgtcgttgccttcacctccctgaccacaagtgctccggggctctttctaagggaggcagctgttctaggcgggaggctggagtctcctgggcctggacgcaccccggggtgtgagtgatgggtatgcctgaaaggaggggaagtggcgcggctttaatcatctgggctagtccccggcgggcctgggggaacagggaaactggaggcggccttaaagcgtcaggatgagacatcgcaagaggagctgcagatactgagcgtgcgccccgggttctcgccgccttctctccgccgagcagcccttcggccaccctttgcccttaaaaatctgcagactgcgcctcctctccgcgggagcgagacctagcaggcccggggctgggcgtgccctcgcctgccacgctgcgcgctgccctcagccgggccgctggggccgtgcagtgcaccgggcacgccgcgccaggctgggggcaggcaccgagcctccgtgggaggtcccgaggcagcttcgcctgctcgccctggctccagccctcacctgccgcagccttagctgagcagccgccgccactgggcgccccccgctccccacttcgccagcgcccgctcctcggctcggcccggggtagtttgtagggacgcagctctccacgtgcgcgactgcgaggctggacgctacgggctcctggaaaggagacaccagcatttgccacaatgctgtcatccactgactttacatttgcttcctgggagcttgtggtccgcgttgaccatcccaatgaagagcagcagaaagacgtcacactgagagtatctggagaccttcatgttggaggagtgatgctcaagttagtagaacagatcaatatatcccaagactggtcagactttgctctttggtgggaacagaagcattgctggcttctgaaaacccactggaccctggacaaatatggggtccaggcagatgcaaagcttctcttcacccctcagcataaaatgctgcgccttcgtctgccgaatttgaagatggtgaggttgcgagtcagcttctcagctgtggtttttaaagctgtcagtgatatctgcaaaatcctgaatattagaagatcagaagagctttccttgttaaagccgtctggtgactattttaagaagaagaagaaaaaagacaaaaataataaggaacccataattgaagatattctaaacctggagagttctccaacagcttcaggttcatcagtaagtcctggtttatacagtaaaaccatgacccctatatatgaccccatcaatggaacaccagcatcatccaccatgacttggttcagtgacagccctttgacggaacaaaactgcagcatcctcgcattcagccaacccccccagtccccagaagcacttgcggatatgtaccagcctcggtctctggttgataaagccaagctcaatgcaggttggctagactcctcacgctcccttatggaacaaggcatccaagaggatgagcagctgctcttacgatttaaatattattctttcttcgacttgaatcctaaatatgatgctgtccgaataaaccaactctatgagcaagccaggtgggccattctcttagaagaaattgattgcacagaggaagaaatgttgatctttgcagctctacagtaccacattagcaaactgtcgttgtctgctgaaacacaggattttgcaggcgagtccgaggttgatgaaatagaagcggcgctttctaatttggaagtaaccctagaaggtggaaaagcggacagccttttggaggacattactgatatccctaaacttgcagataatctcaaattatttaggcccaagaagttactaccaaaagctttcaaacaatattggtttatctttaaagacacatccatagcatactttaaaaataaggaacttgaacaaggagaaccactagaaaaactaaatcttagaggctgcgaagttgtgcccgatgtaaatgtagcaggaagaaaatttggaatcaagttactaatccctgttgccgatggtatgaatgaaatgtatttgagatgtgaccatgagaatcaatacgcccaatggatggctgcctgcatgttggcatcgaagggcaaaaccatggcagacagctcctaccagccagaggtcctcaacatcctttcatttctgaggatgaaaaacaggaactctgcatctcaggtggcttccagtctcgaaaacatggatatgaacccagaatgttttgtgtcaccacggtgtgcaaagaaacacaaatccaaacagctggccgcccggatcctggaggcgcaccagaacgtggcccagatgcccctggtcgaagccaagctgcggttcatccaggcgtggcagtcactgcctgagtttggcctcacctactaccttgtcagatttaaaggaagcaaaaaagatgacattctgggagtttcatataacaggttgattaaaattgatgcagccaccgggattccagtgacaacatggagattcacaaatatcaaacagtggaatgtaaactgggaaacccggcaggtggtcatcgagtttgaccaaaacgtctttactgctttcacctgcctgagtgcagattgcaagattgtgcacgagtacattggcggctacattttcttgtccacccgctccaaggaccagaatgaaacactcgatgaggacttgttccacaaattgaccggcggtcaggattgaaacaagcacgcgtgctcggctcacaccaacaaggcaagccaaaggcgcccctccccagagggatccctaacgtgcccagcatgtagattctggactaacagacaacatacattcaccgctggtcacccagatcctcattcaaacccactgctggcacatccctttccttactttgccctgtgctaccagccacggaaggagcctctcttgttttttctataaaatgggtaggcaggagaaaagcaggtgccctaagattgctctaaggcccagcatgtggttacagttctctgacttgcagaacctgccaggtgtatggctacaagttatcctcgtgctgatctgtctcattactaagtcaatggagaagacagaaaggtaaaaatcacgtgtagcaagaacaactcttatttcacaaactcaggtatgaaacgaaacgcctgtccttcatggaactgcttttagctcctgtcttttcaaaatggcagagggagttcctacacacactttttccctggaggccaaggtctaggggtagaaaggggaggggtggggctaccaggtagcagttgacaacccaaggtcagaggagtggccctcagtgtcatctgtccacagtgatacctgccaagatgaccactgacccacatctggtcttagtcattggtctcctcagatttctggggccacctgcaagccccattccattcctacagatctctcagccacctgtaagtcctttgtgaagatgtgggtgacacagggggacaggaaaacccatttctcaacccagatccatgtctccactgcttctactctgggttgggattcaggaagacaggcacagtcctctctgttcatagaaacacctgccagtgtcaaggattccagtcaggtgtctatcccaactggtcagggagagaagggcagacccattctcaaagaccaccatgtccaaggtctgacagctccccactggctgcccccacaggggctttaggctggtctgggtcatggggaagcgtccctcttatcgctggtctgtgttctcctggatttggtatctatgttggtacgactcctggccttttatctaaaggactttggcttttgtaaatcacaagccaataatagacttttttctccccctctgttttttgctgtgtcatctctgccttgagactgccttgagacagtgcttgccttgagagagtgagccaattaacagctgcctgaattgtcattttccattttggtttgttagaggtgggaggggtgggttttgagaaggtcaaaagcaataccagaagtaaagggaaatatcagacaatattttattattttttcatagatgttctgccacacaaagaacttggggtgtaaggataaggcaaaagctccaatcccatttttcagttctcctaggatgcacccctcagggagcctggccagagttccgaggctcgtgagcgtcagctgttgctttattttccatcaaagccctctgagaagtgagacctcagcaattccgggagccacatagagacagacttggcaagggaccccctggttctgagccagtagctgccatctggaaattcctcttttagcctctccttagaggtgaatgtgaatgaagcctcccaggcacccgctgaatttctgaggccttgcttaaagctcagaagtggtttaggcatttggaaaatctggttcacatcataaagaacttgatttgaaatgttttctatagaaacaagtgctaagtgtaccgtattatacttgatgttggtcatttctcagtcctatttctcagttctattattttagaacctagtcagttctttaagattataactggtcctacattaaaataatgcttctcgatgtcagattttacctgtttgctgctgagaacatctctgcctaatttaccaaagccagaccttcagttcaacatgcttccttagcttttcatagttgtctgacatttccatgaaaacaaaggaaccaactttgttttaaccaaactttgtttggttacagttttcaggggagcgtttcttccatgacacacagcaacatcccaaagaaataaacaagtgtgacaaaaaaaaaaaaaaacaaacctaaatgctactgttccaaagagcaacttgatggttttttttaatactgagtgcaaaaggtcacccaaattcctatgatgaaattttaaattaatgggcacctttcaacatcatttgcttccttatctacagttgattcagaaatctgcattttttattcttttatatgacttttaagtaaaagatttatatggatttgaaaaaaaaaaaaaaaaa

SEQ ID NO: 6 - protein, 677 aa, Homo sapiens
MLSSTDFTFASWELVVRVDHPNEEQQKDVTLRVSGDLHVGGVMLKLVEQINISQDWSDFALWWEQKHCWLLKTHWTLDKYGVQADAKLLFTPQHKMLRLRLPNLKMVRLRVSFSAVVFKAVSDICKILNIRRSEELSLLKPSGDYFKKKKKKDKNNKEPIIEDILNLESSPTASGSSVSPGLYSKTMTPIYDPINGTPASSTMTWFSDSPLTEQNCSILAFSQPPQSPEALADMYQPRSLVDKAKLNAGWLDSSRSLMEQGIQEDEQLLLRFKYYSFFDLNPKYDAVRINQLYEQARWAILLEEIDCTEEEMLIFAALQYHISKLSLSAETQDFAGESEVDEIEAALSNLEVTLEGGKADSLLEDITDIPKLADNLKLFRPKKLLPKAFKQYWFIFKDTSIAYFKNKELEQGEPLEKLNLRGCEVVPDVNVAGRKFGIKLLIPVADGMNEMYLRCDHENQYAQWMAACMLASKGKTMADSSYQPEVLNILSFLRMKNRNSASQVASSLENMDMNPECFVSPRCAKKHKSKQLAARILEAHQNVAQMPLVEAKLRFIQAWQSLPEFGLTYYLVRFKGSKKDDILGVSYNRLIKIDAATGIPVTTWRFTNIKQWNVNWETRQVVIEFDQNVFTAFTCLSADCKIVHEYIGGYIFLSTRSKDQNETLDEDLFHKLTGGQD

SEQ ID NO: 7 - DNA, 3486 bp, Homo sapiens
  CDS (93)..(3350)
gagccgactgcggctgcgcggggtattcgaatcggcggcggcttctagtttgcggttcaggtttggccgctgccggccagcgtcctctggccatggacaccccggaaaatgtccttcagatgcttgaagcccacatgcagagctacaagggcaatgaccctcttggtgaatgggaaagatacatacagtgggtagaagagaattttcctgagaataaagaatacttgataactttactagaacatttaatgaaggaatttttagataagaagaaataccacaatgacccaagattcatcagttattgtttaaaatttgctgagtacaacagtgacctccatcaattttttgagtttctgtacaaccatgggattggaaccctgtcatcccctctgtacattgcctgggcggggcatctggaagcccaaggagagctgcagcatgccagtgctgtccttcagagaggaattcaaaaccaggctgaacccagagagttcctgcaacaacaatacaggttatttcagacacgcctcactgaaacccatttgccagctcaagctagaacctcagaacctctgcataatgttcaggttttaaatcaaatgataacatcaaaatcaaatccaggaaataacatggcctgcatttctaagaatcagggttcagagctttctggagtgatatcttcagcttgtgataaagagtcaaatatggaacgaagagtgatcacgatttctaaatcagaatattctgtgcactcatctttggcatccaaagttgatgttgagcaggttgttatgtattgcaaggagaagcttattcgtggggaatcagaattttcctttgaagaattgagagcccagaaatacaatcaacggagaaagcatgagcaatgggtaaatgaagacagacattatatgaaaaggaaagaagcaaatgcttttgaagaacagctattaaaacagaaaatggatgaacttcataagaagttgcatcaggtggtggagacatcccatgaggatctgcccgcttcccaggaaaggtccgaggttaatccagcacgtatggggccaagtgtaggctcccagcaggaactgagagcgccatgtcttccagtaacctatcagcagacaccagtgaacatggaaaagaacccaagagaggcacctcctgttgttcctcctttggcaaatgctatttctgcagctttggtgtccccagccaccagccagagcattgctcctcctgttcctttgaaagcccagacagtaacagactccatgtttgcagtggccagcaaagatgctggatgtgtgaataagagtactcatgaattcaagccacagagtggagcagagatcaaagaagggtgtgaaacacataaggttgccaacacaagttcttttcacacaactccaaacacatcactgggaatggttcaggcaacgccatccaaagtgcagccatcacccaccgtgcacacaaaagaagcattaggtttcatcatgaatatgtttcaggctcctacacttcctgatatttctgatgacaaagatgaatggcaatctctagatcaaaatgaagatgcatttgaagcccagtttcaaaaaaatgtaaggtcatctggggcttggggagtcaataagatcatctcttctttgtcatctgcttttcatgtgtttgaagatggaaacaaagaaaattatggattaccacagcctaaaaataaacccacaggagccaggacctttggagaacgctctgtcagcagacttccttcaaaaccaaaggaggaagtgcctcatgctgaagagtttttggatgactcaactgtatggggtattcgctgcaacaaaaccctggcacccagtcctaagagcccaggagacttcacatctgctgcacaacttgcgtctacaccattccacaagcttccagtggagtcagtgcacattttagaagataaagaaaatgtggtagcaaaacagtgtacccaggcgactttggattcttgtgaggaaaacatggtggtgccttcaagggatggaaaattcagtccaattcaagagaaaagcccaaaacaggccttgtcgtctcacatgtattcagcatccttacttcgtctgagccagcctgctgcaggtggggtacttacctgtgaggcagagttgggcgttgaggcttgcagactcacagacactgacgctgccattgcagaagatccaccagatgctattgctgggctccaagcagaatggatgcagatgagttcacttgggactgttgatgctccaaacttcattgttgggaacccatgggatgataagctgattttcaaacttttatctgggctttctaaaccagtgagttcctatccaaatacttttgaatggcaatgtaaacttccagccatcaagcccaagactgaatttcaattgggttctaagctggtctatgtccatcaccttcttggagaaggagcctttgcccaggtgtacgaagctacccagggagatctgaatgatgctaaaaataaacagaaatttgttttaaaggtccaaaagcctgccaacccctgggaattctacattgggacccagttgatggaaagactaaagccatctatgcagcacatgtttatgaagttctattctgcccacttattccagaatggcagtgtattagtaggagagctctacagctatggaacattattaaatgccattaacctctataaaaatacccctgaaaaagtgatgcctcaaggtcttgtcatctcttttgctatgagaatgctttacatgattgagcaagtgcatgactgtgaaatcattcatggagacattaaaccagacaatttcatacttggaaacggatttttggaacaggatgatgaagatgatttatctgctggcttggcactgattgacctgggtcagagtatagatatgaaactttttccaaaaggaactatattcacagcaaagtgtgaaacatctggttttcagtgtgttgagatgctcagcaacaaaccatggaactaccagatcgattactttggggttgctgcaacagtatattgcatgctctttggcacttacatgaaagtgaaaaatgaaggaggagagtgtaagcctgaaggtctttttagaaggcttcctcatttggatatgtggaatgaattttttcatgttatgttgaatattccagattgtcatcatcttccatctttggatttgttaaggcaaaagctgaagaaagtatttcaacaacactatactaacaagattagggccctacgtaataggctaattgtactgctcttagaatgtaagcgttcacgaaaataaaatttggatatagacagtccttaaaaatcacactgtaaatatgaatctgctcactttaaacctgtttttttttcatttattgtttatgtaaatgtttgttaaaaataaatcccatggaatatttccatgtaaaaaa

SEQ ID NO: 8 - protein, 1085 aa, Homo sapiens
MDTPENVLQMLEAHMQSYKGNDPLGEWERYIQWVEENFPENKEYLITLLEHLMKEFLDKKKYHNDPRFISYCLKFAEYNSDLHQFFEFLYNHGIGTLSSPLYIAWAGHLEAQGELQHASAVLQRGIQNQAEPREFLQQQYRLFQTRLTETHLPAQARTSEPLHNVQVLNQMITSKSNPGNNMACISKNQGSELSGVISSACDKESNMERRVITISKSEYSVHSSLASKVDVEQVVMYCKEKLIRGESEFSFEELRAQKYNQRRKHEQWVNEDRHYMKRKEANAFEEQLLKQKMDELHKKLHQVVETSHEDLPASQERSEVNPARMGPSVGSQQELRAPCLPVTYQQTPVNMEKNPREAPPVVPPLANAISAALVSPATSQSIAPPVPLKAQTVTDSMFAVASKDAGCVNKSTHEFKPQSGAEIKEGCETHKVANTSSFHTTPNTSLGMVQATPSKVQPSPTVHTKEALGFIMNMFQAPTLPDISDDKDEWQSLDQNEDAFEAQFQKNVRSSGAWGVNKIISSLSSAFHVFEDGNKENYGLPQPKNKPTGARTFGERSVSRLPSKPKEEVPHAEEFLDDSTVWGIRCNKTLAPSPKSPGDFTSAAQLASTPFHKLPVESVHILEDKENVVAKQCTQATLDSCEENMVVPSRDGKFSPIQEKSPKQALSSHMYSASLLRLSQPAAGGVLTCEAELGVEACRLTDTDAAIAEDPPDAIAGLQAEWMQMSSLGTVDAPNFIVGNPWDDKLIFKLLSGLSKPVSSYPNTFEWQCKLPAIKPKTEFQLGSKLVYVHHLLGEGAFAQVYEATQGDLNDAKNKQKFVLKVQKPANPWEFYIGTQLMERLKPSMQHMFMKFYSAHLFQNGSVLVGELYSYGTLLNAINLYKNTPEKVMPQGLVISFAMRMLYMIEQVHDCEIIHGDIKPDNFILGNGFLEQDDEDDLSAGLALIDLGQSIDMKLFPKGTIFTAKCETSGFQCVEMLSNKPWNYQIDYFGVAATVYCMLFGTYMKVKNEGGECKPEGLFRRLPHLDMWNEFFHVMLNIPDCHHLPSLDLLRQKLKKVFQQHYTNKIRALRNRLIVLLLECKRSRK

SEQ ID NO: 9 - DNA, 2635 bp, Homo sapiens
  CDS (285)..(1679)
ggccgacgcgagcgccgcgcttcgcttcagctgctagctggcccaagggaggcgaccgcggagggtggcgaggggcggccaggacccgcagccccggggccgggccggtccggaccgccagggagggcaggtcagtgggcagatcgcgtccgcgggattcaatctctgcccgctctgataacagtccttttccctggcgctcacttcgtgcctggcacccggctgggcgcctcaagaccgttgtctcttcgatcgcttctttggacttggcgaccatttcagagatgtcttccagaagtaccaaagatttaattaaaagtaagtggggatcgaagcctagtaactccaaatccgaaactacattagaaaaattaaagggagaaattgcacacttaaagacatcagtggatgaaatcacaagtgggaaaggaaagctgactgataaagagagacacagacttttggagaaaattcgagtccttgaggctgagaaggagaagaatgcttatcaactcacagagaaggacaaagaaatacagcgactgagagaccaactgaaggccagatatagtactaccgcattgcttgaacagctggaagagacaacgagagaaggagaaaggagggagcaggtgttgaaagccttatctgaagagaaagacgtattgaaacaacagttgtctgctgcaacctcacgaattgctgaacttgaaagcaaaaccaatacactccgtttatcacagactgtggctccaaactgcttcaactcatcaataaataatattcatgaaatggaaatacagctgaaagatgctctggagaaaaatcagcagtggctcgtgtatgatcagcagcgggaagtctatgtaaaaggacttttagcaaagatctttgagttggaaaagaaaacggaaacagctgctcattcactcccacagcagacaaaaaagcctgaatcagaaggttatcttcaagaagagaagcagaaatgttacaacgatctcttggcaagtgcaaaaaaagatcttgaggttgaacgacaaaccataactcagctgagttttgaactgagtgaatttcgaagaaaatatgaagaaacccaaaaagaagttcacaatttaaatcagctgttgtattcacaaagaagggcagatgtgcaacatctggaagatgataggcataaaacagagaagatacaaaaactcagggaagagaatgatattgctaggggaaaacttgaagaagagaagaagagatccgaagagctcttatctcaggtccagtttctttacacatctctgctaaagcagcaagaagaacaaacaagggtagctctgttggaacaacagatgcaggcatgtactttagactttgaaaatgaaaaactcgaccgtcaacatgtgcagcatcaattgcttgtaattcttaaggagctccgaaaagcaagaaatcaaataacacagttggaatccttgaaacagcttcatgagtttgccatcacagagccattagtcactttccaaggagagactgaaaacagagaaaaagttgccgcctcaccaaaaagtcccactgctgcactcaatgaaagcctggtggaatgtcccaagtgcaatatacagtatccagccactgagcatcgcgatctgcttgtccatgtggaatactgttcaaagtagcaaaataagtatttgttttgatattaaaagattcaatactgtattttctgttagcttgtgggcattttgaattatatatttcacattttgcataaaactgcctatctacctttgacactccagcatgctagtgaatcatgtatcttttaggctgctgtgcatttctcttggcagtgatacctccctgacatggttcatcatcaggctgcaatgacagaatgtggtgagcagcgtctactgagactactaacattttgcactgtcaaaatacttggtgaggaaaagatagctcaggttattgctaatgggttaatgcaccagcaagcaaaatattttatgttttgggggtttgaaaaatcaaagataattaaccaaggatcttaactgtgttcgcattttttatccaagcacttagaaaacctacaatcctaattttgatgtccattgttaagaggtggtgatagatactattttttttttcatattgtatagcggttattagaaaagttggggattttcttgatctttattgctgcttaccattgaaacttaacccagctgtgttccccaactctgttctgcgcacgaaacagtatctgtttgaggcataatcttaagtggccacacacaatgttttctcttatgttatctggcagtaactgtaacttgaattacattagcacattctgcttagctaaaattgttaaaataaactttaataaacccatgtagccctctcatttgattgacagtattttagttatttttggcattcttaaagctgggcaatgtaatgatcagatctttgtttgtctgaacaggtatttttatacatgctttttgtaaaccaaaaacttttaaatttcttcaggttttctaacatgcttaccactgggctactgtaaatgagaaaagaataaaattatttaatgttttaaaaaaaaaaaaaaaaaaaa

SEQ ID NO: 10 - protein, 464 aa, Homo sapiens
MSSRSTKDLIKSKWGSKPSNSKSETTLEKLKGEIAHLKTSVDEITSGKGKLTDKERHRLLEKIRVLEAEKEKNAYQLTEKDKEIQRLRDQLKARYSTTALLEQLEETTREGERREQVLKALSEEKDVLKQQLSAATSRIAELESKTNTLRLSQTVAPNCFNSSINNIHEMEIQLKDALEKNQQWLVYDQQREVYVKGLLAKIFELEKKTETAAHSLPQQTKKPESEGYLQEEKQKCYNDLLASAKKDLEVERQTITQLSFELSEFRRKYEETQKEVHNLNQLLYSQRRADVQHLEDDRHKTEKIQKLREENDIARGKLEEEKKRSEELLSQVQFLYTSLLKQQEEQTRVALLEQQMQACTLDFENEKLDRQHVQHQLLVILKELRKARNQITQLESLKQLHEFAITEPLVTFQGETENREKVAASPKSPTAALNESLVECPKCNIQYPATEHRDLLVHVEYCSK

SEQ ID NO: 11 - DNA, 2765 bp, Homo sapiens
  CDS (329)..(1048)
cggagtctggccgcagtcgcggcagtggtggcttcccatccccaaaaggcgccctccgactccttgcgccgcactgctcgccgggccagtccggaaacgggtcgtggagctccgcaccactcccgctggttcccgaaggcagatcccttctcccgagagttgcgagaaactttcccttgtccccgacgctgcagcggctcgggtaccgtggcagccgcaggtttctgaaccccgggccacgctccccgcgcctcggcttcgcgctcgtgtagatcgttccctctctggttgcacgctggggatcccggacctcgattctgcgggcgagatgcccctgggacacatcatgaggctggacctggagaaaattgccctggagtacatcgtgccctgtctgcacgaggtgggcttctgctacctggacaacttcctgggcgaggtggtgggcgactgcgtcctggagcgcgtcaagcagctgcactgcaccggggccctgcgggacggccagctggcggggccgcgcgccggcgtctccaagcgacacctgcggggcgaccagatcacgtggatcgggggcaacgaggagggctgcgaggccatcagcttcctcctgtccctcatcgacaggctggtcctctactgcgggagccggctgggcaaatactacgtcaaggagaggtctaaggcaatggtggcttgctatccgggaaatggaacaggttatgttcgccacgtggacaaccccaacggtgatggtcgctgcatcacctgcatctactatctgaacaagaattgggatgccaagctacatggtgggatcctgcggatatttccagaggggaaatcattcatagcagatgtggagcccatttttgacagactcctgttcttctggtcagatcgtaggaacccacacgaagtgcagccctcttacgcaaccagatatgctatgactgtctggtactttgatgctgaagaaagggcagaagccaaaaagaaattcaggaatttaactaggaaaactgaatctgccctcactgaagactgaccgtgctctgaaatctgctggccttgttcattttagtaacggttcctgaattctcttaaattctttgagatccaaagatggcctcttcagtgacaacaatctccctgctacttcttgcatccttcacatccctgtcttgtgtgtggtacttcatgttttcttgccaagactgtgttgatcttcagatactctctttgccagatgaagttacttgctaactccagaaattcctgcagacatcctactcggccagcggtttacctgatagattcggtaatactatcaagagaagagcctaggagcacagcgagggaatgaaccttacttgcactttatgtatacttcctgatttgaaaggaggaggtttgaaaagaaaaaaatggaggtggtagatgccacagagaggcatcacggaagccttaacagcaggaaacagagaaatttgtgtcatctgaacaatttccagatgttcttaatccagggctgttggggtttctggagaattatcacaacctaatgacattaatacctctagaaagggctgctgtcatagtgaacaatttataagtgtcccatggggcagacactccttttttcccagtcctgcaacctggattttctgcctcagccccattttgctgaaaataatgactttctgaataaagatggcaacacaattttttctccattttcagttcttacctgggaacctaattccccagaagctaaaaaactagacattagttgttttggttgctttgttggaatggaatttaaatttaaatgaaaggaaaaatatatccctggtagttttgtgttaaccactgataactgtggaaagagctaggtctactgatatacaataaacatgtgtgcatcttgaacaatttgagaggggaggtggagttggaaatgtgggtgttcctgttttttttttttttttttttttagttttcctttttaatgagctcaccctttaacacaaaaaaagcagggtgatgtattttaaaaaaggaagtggaaataaaaaaatctcaaagctatttgagttctcgtctgtccctagcagtctttcttcagctcacttggctctctagatccactgtggttggcagtatgaccagaatcatggaatttgctagaactgtggaagcttctactcctgcagtaagcacagatcgcactgcctcaataacttggtattgagcacgtattttgcaaaagctacttttcctagttttcagtattactttcatgttttaaaaatccctttaatttcttgcttgaaaatcccatgaacattaaagagccagaaatattttcctttgttatgtacggatatatatatatatagtcttccaagatagaagtttactttttcctcttctggttttggaaaatttccagataagacatgtcaccattaattctcaacgactgctctattttgttgtacggtaatagttatcaccttctaaattactatgtaatttactcacttattatgtttattgtcttgtatcctttctctggagtgtaagcacaatgaagacaggaattttgtatatttttaaccaatgcaacatactctcagcacctaaaatagtgccgggaacatagtaagggctcagtaaatacttgttgaataaactcagtctcctacattagcattctaaaaaaaaaaaaaaaaaaaaaaaaaaaaaaaaaaaaaaaaaag

SEQ ID NO: 12 - protein, 239 aa, Homo sapiens
MPLGHIMRLDLEKIALEYIVPCLHEVGFCYLDNFLGEVVGDCVLERVKQLHCTGALRDGQLAGPRAGVSKRHLRGDQITWIGGNEEGCEAISFLLSLIDRLVLYCGSRLGKYYVKERSKAMVACYPGNGTGYVRHVDNPNGDGRCITCIYYLNKNWDAKLHGGILRIFPEGKSFIADVEPIFDRLLFFWSDRRNPHEVQPSYATRYAMTVWYFDAEERAEAKKKFRNLTRKTESALTED

SEQ ID NO: 13 - protein, 10 aa, artificial
  Synthetic peptide
KYNYIASFFI

SEQ ID NO: 14 - protein, 9 aa, artificial
  Synthetic peptide
RYSKDRKDL

SEQ ID NO: 15 - protein, 9 aa, artificial
  Synthetic peptide
FYQVEPSEI

SEQ ID NO: 16 - protein, 9 aa, artificial
  Synthetic peptide
KYELENEEI

SEQ ID NO: 17 - protein, 9 aa, artificial
  Synthetic peptide
DMSNSNDCM

SEQ ID NO: 18 - protein, 9 aa, artificial
  Synthetic peptide
CMRDSSQIL

SEQ ID NO: 19 - protein, 9 aa, artificial
  Synthetic peptide
RMKHIRQAM

SEQ ID NO: 20 - protein, 10 aa, artificial
  Synthetic peptide
QFVRFEATDL

SEQ ID NO: 21 - protein, 10 aa, artificial
  Synthetic peptide
CMPRGYLEVI

SEQ ID NO: 22 - protein, 9 aa, artificial
  Synthetic peptide
CFNSDIVLM

SEQ ID NO: 23 - protein, 10 aa, artificial
  Synthetic peptide
PWADRTARVI

SEQ ID NO: 24 - protein, 10 aa, artificial
  Synthetic peptide
TFTVTGEPLL

SEQ ID NO: 25 - protein, 9 aa, artificial
  Synthetic peptide
GFSLLSAFI

SEQ ID NO: 26 - protein, 10 aa, artificial
  Synthetic peptide
PFMQELVDGL

SEQ ID NO: 27 - protein, 10 aa, artificial
  Synthetic peptide
QMEISFLPEF

SEQ ID NO: 28 - protein, 10 aa, artificial
  Synthetic peptide
LWDVDRKKNM

SEQ ID NO: 29 - protein, 10 aa, artificial
  Synthetic peptide
TWGYNDNSCI

SEQ ID NO: 30 - protein, 9 aa, artificial
  Synthetic peptide
GYNDNSCIF

SEQ ID NO: 31 - protein, 9 aa, artificial
  Synthetic peptide
TFIRYFVAM

SEQ ID NO: 32 - protein, 9 aa, artificial
  Synthetic peptide
ILSHSLWDV

SEQ ID NO: 33 - protein, 9 aa, artificial
  Synthetic peptide
FMQELVDGL

SEQ ID NO: 34 - protein, 9 aa, artificial
  Synthetic peptide
GLYHTGANV

SEQ ID NO: 35 - protein, 10 aa, artificial
  Synthetic peptide
FIYDFCIFGV

SEQ ID NO: 36 - protein, 10 aa, artificial
  Synthetic peptide
LMPELSTFRV

SEQ ID NO: 37 - protein, 10 aa, artificial
  Synthetic peptide
AMAKNRLQFV

SEQ ID NO: 38 - protein, 10 aa, artificial
  Synthetic peptide
GILSHSLWDV

SEQ ID NO: 39 - protein, 10 aa, artificial
  Synthetic peptide
FLPEFGISSA

SEQ ID NO: 40 - protein, 10 aa, artificial
  Synthetic peptide
FLNNHDQPFM

SEQ ID NO: 41 - protein, 10 aa, artificial
  Synthetic peptide
QLTITGKKWL

SEQ ID NO: 42 - protein, 10 aa, artificial
  Synthetic peptide
QWVRPARADF

SEQ ID NO: 43 - protein, 10 aa, artificial
  Synthetic peptide
SWASAEKPYL

SEQ ID NO: 44 - protein, 10 aa, artificial
  Synthetic peptide
YFQTVKHNNI

SEQ ID NO: 45 - protein, 10 aa, artificial
  Synthetic peptide
LMDVFTDVEI

SEQ ID NO: 46 - protein, 10 aa, artificial
  Synthetic peptide
IYCAKSGRKF

SEQ ID NO: 47 - protein, 9 aa, artificial
  Synthetic peptide
KFTGQAVEL

SEQ ID NO: 48 - protein, 9 aa, artificial
  Synthetic peptide
LFDEEFRHL

SEQ ID NO: 49 - protein, 9 aa, artificial
  Synthetic peptide
LYASSKPVM

SEQ ID NO: 50 - protein, 9 aa, artificial
  Synthetic peptide
ILMDVFTDV

SEQ ID NO: 51 - protein, 9 aa, artificial
  Synthetic peptide
VLSGSYSFT

SEQ ID NO: 52 - protein, 9 aa, artificial
  Synthetic peptide
LQSGTYFPV

SEQ ID NO: 53 - protein, 10 aa, artificial
  Synthetic peptide
SLQSGTYFPV

SEQ ID NO: 54 - protein, 10 aa, artificial
  Synthetic peptide
VLLDQGGVKL

SEQ ID NO: 55 - protein, 10 aa, artificial
  Synthetic peptide
KLFQEMCDKV

SEQ ID NO: 56 - protein, 10 aa, artificial
  Synthetic peptide
FLSSVEAQYI

SEQ ID NO: 57 - protein, 10 aa, artificial
  Synthetic peptide
ILSKFTGQAV

SEQ ID NO: 58 - protein, 10 aa, artificial
  Synthetic peptide
VILMDVFTDV

SEQ ID NO: 59 - protein, 10 aa, artificial
  Synthetic peptide
KYGVQADAKL

SEQ ID NO: 60 - protein, 9 aa, artificial
  Synthetic peptide
LYSKTMTPI

SEQ ID NO: 61 - protein, 10 aa, artificial
  Synthetic peptide
KYDAVRINQL

SEQ ID NO: 62 - protein, 10 aa, artificial
  Synthetic peptide
LYEQARWAIL

SEQ ID NO: 63 - protein, 9 aa, artificial
  Synthetic peptide
QYHISKLSL

SEQ ID NO: 64 - protein, 10 aa, artificial
  Synthetic peptide
SYQPEVLNIL

SEQ ID NO: 65 - protein, 9 aa, artificial
  Synthetic peptide
EYIGGYIFL

SEQ ID NO: 66 - protein, 9 aa, artificial
  Synthetic peptide
VMLKLVEQI

SEQ ID NO: 67 - protein, 10 aa, artificial
  Synthetic peptide
CWLLKTHWTL

SEQ ID NO: 68 - protein, 9 aa, artificial
  Synthetic peptide
LFTPQHKML

SEQ ID NO: 69 - protein, 10 aa, artificial
  Synthetic peptide
TMTWFSDSPL

SEQ ID NO: 70 - protein, 9 aa, artificial
  Synthetic peptide
GWLDSSRSL

SEQ ID NO: 71 - protein, 10 aa, artificial
  Synthetic peptide
RFKYYSFFDL

SEQ ID NO: 72 - protein, 10 aa, artificial
  Synthetic peptide
YWFIFKDTSI

SEQ ID NO: 73 - protein, 10 aa, artificial
  Synthetic peptide
QYAQWMAACM

SEQ ID NO: 74 - protein, 9 aa, artificial
  Synthetic peptide
NMDMNPECF

SEQ ID NO: 75 - protein, 9 aa, artificial
  Synthetic peptide
QMPLVEAKL

SEQ ID NO: 76 - protein, 9 aa, artificial
  Synthetic peptide
RFIQAWQSL

SEQ ID NO: 77 - protein, 10 aa, artificial
  Synthetic peptide
AWQSLPEFGL

SEQ ID NO: 78 - protein, 10 aa, artificial
  Synthetic peptide
TWRFTNIKQW

SEQ ID NO: 79 - protein, 9 aa, artificial
  Synthetic peptide
WLLKTHWTL

SEQ ID NO: 80 - protein, 9 aa, artificial
  Synthetic peptide
SLLEDITDI

SEQ ID NO: 81 - protein, 9 aa, artificial
  Synthetic peptide
KLFRPKKLL

SEQ ID NO: 82 - protein, 9 aa, artificial
  Synthetic peptide
KLNLRGCEV

SEQ ID NO: 83 - protein, 9 aa, artificial
  Synthetic peptide
ILLEEIDCT

SEQ ID NO: 84 - protein, 9 aa, artificial
  Synthetic peptide
KQWNVNWET

SEQ ID NO: 85 - protein, 9 aa, artificial
  Synthetic peptide
VVIEFDQNV

SEQ ID NO: 86 - protein, 10 aa, artificial
  Synthetic peptide
QLYEQARWAI

SEQ ID NO: 87 - protein, 10 aa, artificial
  Synthetic peptide
KMLRLRLPNL

SEQ ID NO: 88 - protein, 10 aa, artificial
  Synthetic peptide
LLFTPQHKML

SEQ ID NO: 89 - protein, 10 aa, artificial
  Synthetic peptide
EYLITLLEHL

SEQ ID NO: 90 - protein, 9 aa, artificial
  Synthetic peptide
KYHNDPRFI

SEQ ID NO: 91 - protein, 9 aa, artificial
  Synthetic peptide
LYNHGIGTL

SEQ ID NO: 92 - protein, 9 aa, artificial
  Synthetic peptide
LYIAWAGHL

SEQ ID NO: 93 - protein, 9 aa, artificial
  Synthetic peptide
QYRLFQTRL

SEQ ID NO: 94 - protein, 9 aa, artificial
  Synthetic peptide
MYSASLLRL

SEQ ID NO: 95 - protein, 9 aa, artificial
  Synthetic peptide
VYEATQGDL

SEQ ID NO: 96 - protein, 9 aa, artificial
  Synthetic peptide
SYGTLLNAI

SEQ ID NO: 97 - protein, 9 aa, artificial
  Synthetic peptide
HYTNKIRAL

SEQ ID NO: 98 - protein, 10 aa, artificial
  Synthetic peptide
RYIQWVEENF

SEQ ID NO: 99 - protein, 10 aa, artificial
  Synthetic peptide
EYNSDLHQFF

SEQ ID NO: 100 - protein, 9 aa, artificial
  Synthetic peptide
NMERRVITI

SEQ ID NO: 101 - protein, 9 aa, artificial
  Synthetic peptide
VMYCKEKLI

SEQ ID NO: 102 - protein, 9 aa, artificial
  Synthetic peptide
KMDELHKKL

SEQ ID NO: 103 - protein, 9 aa, artificial
  Synthetic peptide
TYQQTPVNM

SEQ ID NO: 104 - protein, 10 aa, artificial
  Synthetic peptide
EFKPQSGAEI

SEQ ID NO: 105 - protein, 10 aa, artificial
  Synthetic peptide
MFQAPTLPDI

SEQ ID NO: 106 - protein, 10 aa, artificial
  Synthetic peptide
TFGERSVSRL

SEQ ID NO: 107 - protein, 9 aa, artificial
  Synthetic peptide
EFLDDSTVW

SEQ ID NO: 108 - protein, 10 aa, artificial
  Synthetic peptide
VWGIRCNKTL

SEQ ID NO: 109 - protein, 10 aa, artificial
  Synthetic peptide
PWDDKLIFKL

SEQ ID NO: 110 - protein, 9 aa, artificial
  Synthetic peptide
LMERLKPSM

SEQ ID NO: 111 - protein, 10 aa, artificial
  Synthetic peptide
LYKNTPEKVM

SEQ ID NO: 112 - protein, 9 aa, artificial
  Synthetic peptide
NFILGNGFL

SEQ ID NO: 113 - protein, 10 aa, artificial
  Synthetic peptide
DMKLFPKGTI

SEQ ID NO: 114 - protein, 10 aa, artificial
  Synthetic peptide
VYCMLFGTYM

SEQ ID NO: 115 - protein, 10 aa, artificial
  Synthetic peptide
LFRRLPHLDM

SEQ ID NO: 116 - protein, 10 aa, artificial
  Synthetic peptide
MWNEFFHVML

SEQ ID NO: 117 - protein, 10 aa, artificial
  Synthetic peptide
VFQQHYTNKI

SEQ ID NO: 118 - protein, 9 aa, artificial
  Synthetic peptide
QLGSKLVYV

SEQ ID NO: 119 - protein, 9 aa, artificial
  Synthetic peptide
MLFGTYMKV

SEQ ID NO: 120 - protein, 9 aa, artificial
  Synthetic peptide
DMWNEFFHV

SEQ ID NO: 121 - protein, 9 aa, artificial
  Synthetic peptide
TVTDSMFAV

SEQ ID NO: 122 - protein, 9 aa, artificial
  Synthetic peptide
RMLYMIEQV

SEQ ID NO: 123 - protein, 9 aa, artificial
  Synthetic peptide
GLFRRLPHL

SEQ ID NO: 124 - protein, 9 aa, artificial
  Synthetic peptide
YMIEQVHDC

SEQ ID NO: 125 - protein, 9 aa, artificial
  Synthetic peptide
LLSGLSKPV

SEQ ID NO: 126 - protein, 9 aa, artificial
  Synthetic peptide
YLITLLEHL

SEQ ID NO: 127 - protein, 9 aa, artificial
  Synthetic peptide
FLYNHGIGT

SEQ ID NO: 128 - protein, 9 aa, artificial
  Synthetic peptide
KMDELHKKL

SEQ ID NO: 129 - protein, 9 aa, artificial
  Synthetic peptide
KLVYVHHLL

SEQ ID NO: 130 - protein, 9 aa, artificial
  Synthetic peptide
WVNEDRHYM

SEQ ID NO: 131 - protein, 9 aa, artificial
  Synthetic peptide
RLTDTDAAI

SEQ ID NO: 132 - protein, 9 aa, artificial
  Synthetic peptide
ALIDLGQSI

SEQ ID NO: 133 - protein, 9 aa, artificial
  Synthetic peptide
LIFKLLSGL

SEQ ID NO: 134 - protein, 9 aa, artificial
  Synthetic peptide
VMYCKEKLI

SEQ ID NO: 135 - protein, 9 aa, artificial
  Synthetic peptide
FITYCLKFA

SEQ ID NO: 136 - protein, 9 aa, artificial
  Synthetic peptide
KLPVESVHI

SEQ ID NO: 137 - protein, 9 aa, artificial
  Synthetic peptide
IMNMFQAPT

SEQ ID NO: 138 - protein, 10 aa, artificial
  Synthetic peptide
FQLGSKLVYV

SEQ ID NO: 139 - protein, 10 aa, artificial
  Synthetic peptide
FLDDSTVWGI

SEQ ID NO: 140 - protein, 10 aa, artificial
  Synthetic peptide
LLGEGAFAQV

SEQ ID NO: 141 - protein, 10 aa, artificial
  Synthetic peptide
KLLSGLSKPV

SEQ ID NO: 142 - protein, 10 aa, artificial
  Synthetic peptide
GEWERYIQWV

SEQ ID NO: 143 - protein, 10 aa, artificial
  Synthetic peptide
KLFPKGTIFT

SEQ ID NO: 144 - protein, 10 aa, artificial
  Synthetic peptide
CMLFGTYMKV

SEQ ID NO: 145 - protein, 10 aa, artificial
  Synthetic peptide
KLIFKLLSGL

SEQ ID NO: 146 - protein, 10 aa, artificial
  Synthetic peptide
VLTCEAELGV

SEQ ID NO: 147 - protein, 10 aa, artificial
  Synthetic peptide
NLYKNTPEKV

SEQ ID NO: 148 - protein, 10 aa, artificial
  Synthetic peptide
FLYNHGIGTL

SEQ ID NO: 149 - protein, 10 aa, artificial
  Synthetic peptide
QLLKQKMDEL

SEQ ID NO: 150 - protein, 10 aa, artificial
  Synthetic peptide
QLMERLKPSM

SEQ ID NO: 151 - protein, 10 aa, artificial
  Synthetic peptide
KLPVESVHIL

SEQ ID NO: 152 - protein, 10 aa, artificial
  Synthetic peptide
WMQMSSLGTV

SEQ ID NO: 153 - protein, 10 aa, artificial
  Synthetic peptide
FIMNMFQAPT

SEQ ID NO: 154 - protein, 10 aa, artificial
  Synthetic peptide
RLFQTRLTET

SEQ ID NO: 155 - protein, 10 aa, artificial
  Synthetic peptide
WNYQIDYFGV

SEQ ID NO: 156 - protein, 10 aa, artificial
  Synthetic peptide
GMVQATPSKV

SEQ ID NO: 157 - protein, 10 aa, artificial
  Synthetic peptide
DLLRQKLKKV

SEQ ID NO: 158 - protein, 10 aa, artificial
  Synthetic peptide
VYVKGLLAKI

SEQ ID NO: 159 - protein, 10 aa, artificial
  Synthetic peptide
QYPATEHRDL

SEQ ID NO: 160 - protein, 10 aa, artificial
  Synthetic peptide
EMEIQLKDAL

SEQ ID NO: 161 - protein, 9 aa, artificial
  Synthetic peptide
QMQACTLDF

SEQ ID NO: 162 - protein, 10 aa, artificial
  Synthetic peptide
VYVKGLLAKI

SEQ ID NO: 163 - protein, 10 aa, artificial
  Synthetic peptide
QYPATEHRDL

SEQ ID NO: 164 - protein, 10 aa, artificial
  Synthetic peptide
EMEIQLKDAL

SEQ ID NO: 165 - protein, 9 aa, artificial
  Synthetic peptide
QMQACTLDF

SEQ ID NO: 166 - protein, 9 aa, artificial
  Synthetic peptide
GLLAKIFEL

SEQ ID NO: 167 - protein, 9 aa, artificial
  Synthetic peptide
QLLVILKEL

SEQ ID NO: 168 - protein, 9 aa, artificial
  Synthetic peptide
ALLEQLEET

SEQ ID NO: 169 - protein, 9 aa, artificial
  Synthetic peptide
KQQEEQTRV

SEQ ID NO: 170 - protein, 9 aa, artificial
  Synthetic peptide
YLQEEKQKC

SEQ ID NO: 171 - protein, 9 aa, artificial
  Synthetic peptide
TQLESLKQL

SEQ ID NO: 172 - protein, 10 aa, artificial
  Synthetic peptide
LLYSQRRADV

SEQ ID NO: 173 - protein, 10 aa, artificial
  Synthetic peptide
KLTDKERHRL

SEQ ID NO: 174 - protein, 10 aa, artificial
  Synthetic peptide
ALLEQQMQAC

SEQ ID NO: 175 - protein, 10 aa, artificial
  Synthetic peptide
LLSQVQFLYT

SEQ ID NO: 176 - protein, 9 aa, artificial
  Synthetic peptide
IMRLDLEKI

SEQ ID NO: 177 - protein, 9 aa, artificial
  Synthetic peptide
GFCYLDNFL

SEQ ID NO: 178 - protein, 10 aa, artificial
  Synthetic peptide
SFLLSLIDRL

SEQ ID NO: 179 - protein, 10 aa, artificial
  Synthetic peptide
YYVKERSKAM

SEQ ID NO: 180 - protein, 10 aa, artificial
  Synthetic peptide
NWDAKLHGGI

SEQ ID NO: 181 - protein, 9 aa, artificial
  Synthetic peptide
IFPEGKSFI

SEQ ID NO: 182 - protein, 9 aa, artificial
  Synthetic peptide
SFIADVEPI

SEQ ID NO: 183 - protein, 9 aa, artificial
  Synthetic peptide
RYAMTVWYF

SEQ ID NO: 184 - protein, 9 aa, artificial
  Synthetic peptide
FLLSLIDRL

SEQ ID NO: 185 - protein, 9 aa, artificial
  Synthetic peptide
YLDNFLGEV

SEQ ID NO: 186 - protein, 9 aa, artificial
  Synthetic peptide
YIVPCLHEV

SEQ ID NO: 187 - protein, 9 aa, artificial
  Synthetic peptide
KLHGGILRI

SEQ ID NO: 188 - protein, 9 aa, artificial
  Synthetic peptide
QLAGPRAGV

SEQ ID NO: 189 - protein, 9 aa, artificial
  Synthetic peptide
CVLERVKQL

SEQ ID NO: 190 - protein, 10 aa, artificial
  Synthetic peptide
CLHEVGFCYL

SEQ ID NO: 191 - protein, 10 aa, artificial
  Synthetic peptide
FLGEVVGDCV

SEQ ID NO: 192 - protein, 10 aa, artificial
  Synthetic peptide
SLIDRLVLYC

SEQ ID NO: 193 - protein, 10 aa, artificial
  Synthetic peptide
FLLSLIDRLV

SEQ ID NO: 194 - protein, 10 aa, artificial
  Synthetic peptide
YLNKNWDAKL

SEQ ID NO: 195 - protein, 11 aa, artificial
  Synthetic peptide
LMPELSTFRVL

SEQ ID NO: 196 - protein, 12 aa, artificial
  Synthetic peptide
NMFCSTSGTEQL

SEQ ID NO: 197 - protein, 12 aa, artificial
  Synthetic peptide
AMKKYELENEEI

SEQ ID NO: 198 - protein, 11 aa, artificial
  Synthetic peptide
RYIAKRQLSHL

SEQ ID NO: 199 - protein, 11 aa, artificial
  Synthetic peptide
LYHTGANVESF

SEQ ID NO: 200 - protein, 11 aa, artificial
  Synthetic peptide
RYFVAMKKYEL

SEQ ID NO: 201 - protein, 12 aa, artificial
  Synthetic peptide
IYDFCIFGVPEI

SEQ ID NO: 202 - DNA, 20 bp, Artificial
  primer
tggcactggaagaagatcaa

SEQ ID NO: 203 - DNA, 23 bp, Artificial
  primer
tcaccacaactttgttgtttgtt

SEQ ID NO: 204 - DNA, 20 bp, Artificial
  primer
cagatccaggtgcctctgac

SEQ ID NO: 205 - DNA, 21 bp, Artificial
  primer
tcaggtaatccaaccaccttg

SEQ ID NO: 206 - DNA, 20 bp, Artificial
  primer
tgaatttctgaggccttgct

SEQ ID NO: 207 - DNA, 20 bp, Artificial
  primer
tgttctcagcagcaaacagg

SEQ ID NO: 208 - DNA, 20 bp, Artificial
  primer
ttatctgctggcttggcact

SEQ ID NO: 209 - DNA, 21 bp, Artificial
  primer
gcttttgccttaacaaatcca

SEQ ID NO: 210 - DNA, 22 bp, Artificial
  primer
tgtccattgttaagaggtggtg

SEQ ID NO: 211 - DNA, 20 bp, Artificial
  primer
tgagagggctacatgggttt

SEQ ID NO: 212 - DNA, 20 bp, Artificial
  primer
catccctgtcttgtgtgtgg

SEQ ID NO: 213 - DNA, 20 bp, Artificial
  primer
ccaacagccctggattaaga

(19) United States
(12) Patent Application Publication
(10) Pub. No.: US 2010/0015101 A1
(43) Pub. Date: Jan. 21, 2010

(54) NOVEL TUMOR ANTIGEN PEPTIDES

(76) Inventors: Noriyuki Sato, Hokkaido (JP); Toshihiko Torigoe, Hokkaido (JP); Yoshihiko Hirohashi, Hokkaido (JP); Kenji Harada, Osaka-fu (JP)

Correspondence Address: BIRCH STEWART KOLASCH & BIRCH, PO BOX 747, FALLS CHURCH, VA, 22040-0747, US

(21) Appl. No.: 12/294,899

(22) PCT Filed: Mar. 27, 2007
(86) PCT No.: PCT/JP2007/056279

§ 371 Date: Sep. 26, 2008

(30) Foreign Application Priority Data
Mar. 28, 2006 (JP) 2006-086820

(51) Int. Cl. A61K 35/12; C12N 9/10; A61K 38/45; C07H 21/04; A61K 31/7088; C12N 5/02; C07K 16/00; C12Q 1/48; A61P 35/04; C12Q 1/68

(52) U.S. Cl. 424/93.7; 435/193; 424/94.5; 536/23.2; 514/44 R; 435/375; 424/93.71; 530/387.9; 435/15; 536/24.3; 536/24.33; 435/6

(57) ABSTRACT

The invention relates to novel tumor antigen proteins and peptides derived therefrom and use of the same in the filed of cancer immunity. Specifically, the inventions relates to a peptide which comprises a partial peptide derived from Lengsin, BJ-TSA-9, C20orf42, BUB1, C10orf3 or HIFPH3 and is capable of binding to an HLA antigen and is recognized by a CTL, and a pharmaceutical composition comprising the peptide and a pharmaceutically acceptable carrier.

### TECHNICAL FIELD

The invention relates to novel tumor antigen proteins and peptides derived therefrom and use of the same in the field of cancer immunity.

### BACKGROUND ART

It is known that the immune system, particularly T cells, plays an important role in the elimination of cancer (tumor) by a living body. Indeed, infiltration of lymphocytes exhibiting cytotoxic activity on cancer cells in human cancer foci has been observed (Non-patent literature 1), and cytotoxic T lymphocytes (CTLs) recognizing autologous tumor cells have been isolated from melanomas without great difficulties ((Non-patent literature 2, 3, 4). In addition, the results of clinical treatment of melanomas by transfer of the CTLs also suggested the importance of T cells in cancer elimination (Non-patent literature 5).

Although the target molecules of CTLs attacking autologous tumor cells had long been unclear, such molecules have become clearer gradually along the advance in immunology and molecular biology in recent years. Specifically, it has been revealed that CTLs recognize a complex between a peptide, called cancer antigen peptide, and a major histocompatibility complex class I antigen (MHC class I antigen, also referred to as HLA antigen) through the T cell receptors (TCRs), and thereby attacking autologous tumor cells.

Cancer antigen peptides are generated by intracellular proteasomal degradation of cancer-specific antigen proteins after synthesis in cells. The cancer antigen peptides thus generated bind to MHC class I antigens (HLA antigens) in endoplasmic reticulum to form complexes, and the complexes are transported to the cell surface to be presented as an antigen. Antigen-specific CTLs recognize the complex presented as an antigen, and exhibits anti-cancer effects through the cytotoxic action or production of lymphokines. As a consequence of elucidation of a series of the actions, it has become possible to treat cancer by using cancer antigen protein or a cancer antigen peptide as a so-called cancer vaccine to enhance cancer-specific CTLs in the body of a cancer patient.

As a tumor antigen protein, T. Boon et al. identified a protein named MAGE from human melanoma cells for the first time in 1991 (Non-patent literature 6). Subsequently, several additional tumor antigen proteins have been identified mainly from melanoma cells.

In order to apply tumor antigen proteins and peptides to tumor therapy or diagnostics, it is necessary to identify novel ones which can be applied widely to adenocarcinoma, such as lung cancer, which occurs much more frequently than melanoma.

Lengsin (Glutamate-ammonia ligase (glutamine synthase) domain containing 1 (GLULD1) UniGene Hs.149585) was identified as a novel gene highly expressing in human lens and has been reported to be a possible member of a glutamine synthase family from the similarity of the amino acid sequence. The physiological function of the gene, however, is still unknown (Non-patent literature 7).

BJ-TSA-9 (Hypothetical protein MGC14128) (UniGene Hs.379821) is a novel gene cloned in The National Institutes of Health Mammalian Gene Collection (MGC) Program, and its physiological function is still unknown (Non-patent literature 8).

C20orf42 (or URP1, Kindlerin) was cloned as a gene highly expressing in lung cancer and colon cancer (Non-patent literature 9), and has a high similarity to C. elegans UNC-112. It has been reported that C20orf42 interacts with Integrin as UNC-112 does and the expression of C20orf42 is induced by TGF-β (Non-patent literature 10). C20orf42 was also identified as Kindlerin gene which is mutated in Kindler syndrome, a rare autosomal recessive dermatosis (Non-patent literature 11, 12).

BUB1 is Serine/threonine Kinase involved in spindle checkpoint of cell cycle M phase and lack of functional BUB1 results in abnormal chromosome segregation. In addition, BUB1 phosphorylates Cdc2 and suppresses an ubiquitin ligase activity of APC/C^{c d c 2 0}, resulting in prevention of abnormal chromosome segregation (Non-patent literature 13). Decrease of expression and mutation of BUB1 have been reported in cancer tissue (Non-patent literature 14, 15).

C10orf3 is a novel gene cloned in The National Institutes of Health Mammalian Gene Collection (MGC) Program and its physiological function is still unknown (Non-patent literature 16).

HIFPH3 (egl nine homolog3, EGLN3) was cloned as one of three molecules having a similar activity to egl9, which was identified as a dioxygenase regulating the activity of Hypoxia-inducible factor1 (HIF1) in C. elegans (Non-patent literature 17). It is considered to regulate activities under hypoxic condition through HIF1, but differences between the three egl nine homologs is not known.
Non-patent literature 1: Arch. Surg., 126:200 (1990)
Non-patent literature 2: Immunol. Today, 8:385 (1987)
Non-patent literature 3: J. Immunol., 138:989 (1987)
Non-patent literature 4: Int. J. Cancer, 52:52 (1992)
Non-patent literature 5: J. Natl. Cancer. Inst., 86:1159 (1994)
Non-patent literature 6: Science, 254, 1643-1647 (1991)
Non-patent literature 7: Mol. Vis. Jun. 15; 8:185-95 (2002)
Non-patent literature 8: Proc. Natl. Acad. Sci. USA., 99(26):16899-903 (2002)
Non-patent literature 9: Biochim. Biophys. Acta 1637: 207-216 (2003)
Non-patent literature 10: J. Biol. Chem., February 20; 279(8):6824-33 (2004)
Non-patent literature 11: Hum. Molec. Genet., 12: 925-935 (2003)
Non-patent literature 12: Am. J. Hum. Genet., 73: 174-187 (2003)
Non-patent literature 13: Mol. Cell., November 5; 16(3):387-97 (2004)
Non-patent literature 14: Oncogene July 11; 21(30):4673-9 (2002)
Non-patent literature 15: Leuk. Lymphoma. February; 43(2):385-91 (2002)
Non-patent literature 16: Proc. Natl. Acad. Sci. USA., December 24; 99(26):16899-903 (2002)
Non-patent literature 17: Cell 107: 43-54 (2001)

### DISCLOSURE OF INVENTION

### Problem to be Solved by the Invention

An object of the invention is to provide a novel tumor antigen protein and peptide as well as use of the same in the field of cancer immunity.

### Means for Solving the Problem

The inventors intensively tried to find genes showing increase of expression level or frequency in cancer tissues compared to normal tissues and selected six genes, Lengsin, BJ-TSA-9, C20orf42, BUB1, C10orf3 and HIFPH3 genes, and thus those genes and products expressed therefrom (i.e., proteins) were found to be useful as a disease marker for cancer.

Then, the inventors selected partial peptides possible to bind to an HLA molecule from the amino acid sequence of the proteins encoded by Lengsin, BJ-TSA-9, C20orf42, BUB1, C10orf3 and HIFPH3 genes (Lengsin, BJ-TSA-9, C20orf42, BUB1, C10orf3 and HIFPH3) and examined their biding affinities to an HLA molecule and CTL-inducing activities. As a result, it was found that those six proteins (especially BJ-TSA-9, C20orf42 and C10orf3) were tumor antigen proteins and the partial peptides derived therefrom were tumor antigen peptides. Accordingly, Lengsin, BJ-TSA-9, C20orf42, BUB1, C10orf3 and HIFPH3 and the peptides derived therefrom are considered to be useful as cancer vaccines for various cancers showing expression (particularly increase of expression) of those proteins.

The present invention was accomplished from the findings as described above.

Accordingly, the present invention relates to the followings:

(1) A peptide which comprises a partial peptide derived from Lengsin, BJ-TSA-9, C20orf42, BUB1, C10orf3 or HIFPH3 and is capable of binding to an HLA antigen and is recognized by a CTL;
(2) The peptide of (1) above, wherein the HLA antigen is HLA-A24 or HLA-A2 antigen;
(3) The peptide of (2) above, which comprises the amino acid sequence of any one of SEQ ID NOS: 13 to 201;
(4) A peptide which comprises an amino acid sequence which is the same as the amino acid sequence of any one of SEQ ID NOS: 13 to 31, 42 to 49, 59 to 78, 89 to 117, 158 to 165, 176 to 183, and 195 to 201 except that the amino acid at position 2 is substituted by tyrosine, phenylalanine, methionine or tryptophan, and/or the C terminal amino acid by phenylalanine, leucine, isoleucine, tryptophan or methionine, and is capable of binding to HLA-A24 antigen and is recognized by a CTL;
(5) A peptide which comprises an amino acid sequence which is the same as the amino acid sequence of any one of SEQ ID NOS:32 to 41, 50 to 58, 79 to 88, 118 to 157, 166 to 175, and 184 to 194 except that the amino acid at position 2 is substituted by leucine, methionine, valine, isoleucine or glutamine and/or the C terminal amino acid by valine or leucine, and is capable of binding to HLA-A2 antigen and is recognized by a CTL;
(6) An epitope peptide which comprises the peptide of any one of (1) to (5) above;
(7) A pharmaceutical composition which comprises the peptide of any one of (1) to (6) above and a pharmaceutically acceptable carrier;
(8) A nucleic acid which comprises a polynucleotide encoding the peptide of any one of (1) to (6) above;
(9) A pharmaceutical composition which comprises the nucleic acid of (8) above and a pharmaceutically acceptable carrier;
(10) A pharmaceutical composition which comprises Lengsin, BJ-TSA-9, C20orf42, BUB1, C10orf3 or HIFPH3 and a pharmaceutically acceptable carrier;
(11) The pharmaceutical composition of (10) above, wherein Lengsin comprises the amino acid sequence of SEQ ID NO: 2;
(12) The pharmaceutical composition of (10) above, wherein BJ-TSA-9 comprises the amino acid sequence of SEQ ID NO: 4;
(13) The pharmaceutical composition of (10) above, wherein C20orf42 comprises the amino acid sequence of SEQ ID NO: 6;
(14) The pharmaceutical composition of (10) above, wherein BUB1 comprises the amino acid sequence of SEQ ID NO: 8;
(15) The pharmaceutical composition of (10) above, wherein C10orf3 comprises the amino acid sequence of SEQ ID NO: 10;
(16) The pharmaceutical composition of (10) above, wherein HIFPH3 comprises the amino acid sequence of SEQ ID NO: 12;
(17) A pharmaceutical composition which comprises a nucleic acid comprising a polynucleotide encoding Lengsin, BJ-TSA-9, C20orf42, BUB1, C10orf3 or HIFPH3 and a pharmaceutically acceptable carrier;
(18) The pharmaceutical composition of (17) above, wherein the polynucleotide encoding Lengsin comprises the base sequence of SEQ ID NO: 1, or encodes the amino acid sequence of SEQ ID NO: 2;
(19) The pharmaceutical composition of (17) above, wherein the polynucleotide encoding BJ-TSA-9 comprises the base sequence of SEQ ID NO: 3, or encodes the amino acid sequence of SEQ ID NO: 4;
(20) The pharmaceutical composition of (17) above, wherein the polynucleotide encoding C20orf42 comprises the base sequence of SEQ ID NO: 5, or encodes the amino acid sequence of SEQ ID NO: 6;
(21) The pharmaceutical composition of (17) above, wherein the polynucleotide encoding BUB1 comprises the base sequence of SEQ ID NO: 7, or encodes the amino acid sequence of SEQ ID NO: 8;
(22) The pharmaceutical composition of (17) above, wherein the polynucleotide encoding C10orf3 comprises the base sequence of SEQ ID NO: 9, or encodes the amino acid sequence of SEQ ID NO: 10;
(23) The pharmaceutical composition of (17) above, wherein the polynucleotide encoding HIFPH3 comprises the base sequence of SEQ ID NO: 11, or encodes the amino acid sequence of SEQ ID NO: 12;
(24) A method of preparing an antigen presenting cell, wherein a cell having an antigen-presenting ability is brought into contact in vitro with any one of:
(a) the peptide of any one of (1) to (6) above,
(b) the nucleic acid of (8) above,
(c) Lengsin, BJ-TSA-9, C20orf42, BUB1, C10orf3 or HIFPH3, and
(d) a nucleic acid comprising a polynucleotide encoding Lengsin, BJ-TSA-9, C20orf42, BUB1, C10orf3 or HIFPH3;
(25) An antigen presenting cell prepared by the method of (24) above;
(26) A pharmaceutical composition which comprises the antigen presenting cell of (25) above and a pharmaceutically acceptable carrier;
(27) A method of inducing a CTL, wherein peripheral blood lymphocytes are brought into contact in vitro with any one of:
(a) the peptide of any one of (1) to (6) above,
(b) the nucleic acid of (8) above,
   (c) Lengsin, BJ-TSA-9, C20orf42, BUB1, C10orf3 or HIFPH3, and
   (d) a nucleic acid comprising a polynucleotide encoding Lengsin, BJ-TSA-9, C20orf42, BUB1, C10orf3 or HIFPH3;
   (28) A CTL induced by the method of (27) above;
   (29) A pharmaceutical composition which comprises the CTL of (28) above and a pharmaceutically acceptable carrier;
   (30) The pharmaceutical composition of (7), (9) to (23), (26), or (29) above, which is used as an inducer of CTL;
   (31) The pharmaceutical composition of (7), (9) to (23), (26), or (29) above, which is used as a cancer vaccine;
   (32) An antibody which specifically binds to the peptide of any one of (1) to (5) above;
   (33) An HLA monomer, HLA dimer, HLA tetramer or HLA pentamer which comprises the peptide of any one of (1) to (5) above and an HLA antigen;
   (34) A reagent for detecting a CTL specific to a tumor antigen peptide derived from Lengsin, BJ-TSA-9, C20orf42, BUB1, C10orf3 or HIFPH3, which comprises as a component the HLA monomer, HLA dimer, HLA tetramer or HLA pentamer of (33) above;
   (35) A disease marker consisting of a polynucleotide and/or a complementary polynucleotide thereof, wherein the polynucleotide comprises at least 15 contiguous nucleotides from the base sequence of Lengsin, BJ-TSA-9, C20orf42, BUB1, C10orf3 or HIFPH3 gene;
   (36) The disease marker of (35) above, which is used as a probe or primer for detecting cancer;
   (37) The disease marker of (35) or (36) above, wherein the disease marker derived from Lengsin gene is used for lung adenocarcinoma, lung squamous cell carcinoma or gastric cancer;
   (38) The disease marker of (35) or (36) above, wherein the disease marker derived from BJ-TSA-9 gene is used for leukemia, lung adenocarcinoma, lung squamous cell carcinoma, small cell lung cancer, oral cancer, gastric cancer, pancreas cancer or lymphoma;
   (39) The disease marker of (35) or (36) above, wherein the disease marker derived from C20orf42 gene is used for lung squamous cell carcinoma, lung adenocarcinoma, liver cancer, gastric cancer, leukemia, malignant lymphoma tissues, rectal cancer, colon cancer or pancreas cancer;
   (40) The disease marker of (35) or (36) above, wherein the disease marker derived from BUB1 gene is used for breast cancer, lung adenocarcinoma, lung squamous cell carcinoma, ovarian cancer, oral squamous cell carcinoma, renal cancer, large bowel cancer (colon cancer, rectal cancer), gastric cancer, pancreas cancer, liver cancer, leukemia, lymphoma or melanoma;
   (41) The disease marker of (35) or (36) above, wherein the disease marker derived from C10orf3 gene is used for breast cancer, colon cancer, rectal cancer, renal cancer, gastric cancer, ovarian cancer, liver cancer, pancreas cancer, lung squamous cell carcinoma, lung adenocarcinoma, small cell lung cancer or melanoma;
   (42) The disease marker of (35) or (36) above, wherein the disease marker derived from HIFPH3 gene is used for breast cancer, colon cancer, gastric cancer, renal cancer, pancreas cancer, liver cancer, lung adenocarcinoma or lung squamous cell carcinoma;
   (43) A method for detecting cancer which comprises the following steps (a), (b) and (c):

(a) allowing RNA prepared from a biological sample of a test subject or complementary polynucleotides transcribed therefrom to hybridize with the disease marker of any one of (35) to (42) above;

b) detecting RNA prepared from the biological sample or complementary polynucleotides transcribed therefrom hybridized with the disease marker by using the disease marker as an indicator; and

(c) determining whether or not the test subject has cancer based on the result of the detection in (b);

(44) The method of (43) above, wherein the test subject is determined to have cancer in the step (C) when the result of the detection from the test subject is compared with that from a healthy subject and the level of hybridization to the disease marker observed in the test subject is higher than that observed in the healthy subject;
(45) A disease marker for cancer which comprises an antibody specifically recognizing Lengsin, BJ-TSA-9, C20orf42, BUB1, C10orf3 or HIFPH3;
(46) The disease marker of (45) above, which is used as a probe for detecting cancer;
(47) A method for detecting cancer which comprises the following steps (a), (b) and (c):

(a) allowing proteins prepared from a biological sample of a test subject to bind to the disease marker of (45) or (46) above;

b) detecting proteins prepared from the biological sample or partial peptides derived therefrom bound to the disease marker by using the disease marker as an indicator; and

(c) determining whether or not the test subject has cancer based on the result of the detection in (b);

(48) The method of (47) above, wherein the test subject is determined to have cancer in the step (C) when the result of the detection from the test subject is compared with that from a healthy subject and the level of binding to the disease marker observed in the test subject is higher than that observed in the healthy subject.

### EFFECTS OF THE INVENTION

The novel tumor antigen peptides, the nucleic acids encoding the same, and the like of the present invention can be useful as cancer vaccines. Further, the tumor antigen peptides are also useful as components of HLA tetramers and the like to detect CTLs.

### BEST MODE FOR CARRYING OUT THE INVENTION

Abbreviations for amino acids, (poly)peptides, (poly)nucleotides and the like used herein, follow rules of IUPAC-IUB (IUPAC-IUB Communication on Biological Nomenclature, Eur. J. Biochem., 138: 9 (1984)), “Guidelines for preparing the specification containing a base sequence or an amino acid sequence” (Japan Patent Office), and symbols commonly used in this field.

### 1) Protein of the Present Invention

The proteins of the present invention are Lengsin, BJ-TSA-9, C20orf42, BUB1, C10orf3 and HIFPH3.

The protein of the present invention Lengsin comprises the amino acid sequence of SEQ ID NO: 2 or an amino acid sequence similar to the aforementioned amino acid sequence. The protein Lengsin of the invention may be a protein originated from natural source (e.g., a lung adenocarcinoma cell line 1-87) or a recombinant protein. Here, the amino acid sequence of SEQ ID NO: 2 is registered with the GenBank database under Accession No. NM_{—}016571, Accession No. NP_{—}057655, and represents human Lengsin (Glutamate-ammonia ligase (glutamine synthase) domain containing 1, also referred to as GLULD1). The human Lengsin was disclosed in Non-patent literature 7 (Mol. Vis. Jun. 15; 8:185-95 (2002)).

The protein of the present invention BJ-TSA-9 comprises the amino acid sequence of SEQ ID NO: 4 or an amino acid sequence similar to the aforementioned amino acid sequence. The protein BJ-TSA-9 of the invention may be a protein originated from natural source (e.g., a lung adenocarcinoma cell line 1-87) or a recombinant protein. Here, the amino acid sequence of SEQ ID NO: 4 is registered with the GenBank database under Accession No. NM_{—}032899, Accession No. NP_{—}116288, and represents human BJ-TSA-9 (Hypothetical protein MGC14128). The human BJ-TSA-9 was disclosed in Non-patent literature 8 (Proc. Natl. Acad. Sci. USA., 99(26):16899-903 (2002)).

The protein of the present invention C20orf42 comprises the amino acid sequence of SEQ ID NO: 6 or an amino acid sequence similar to the aforementioned amino acid sequence. The protein C20orf42 of the invention may be a protein originated from natural source (e.g., a colon cancer cell line SW480) or a recombinant protein. Here, the amino acid sequence of SEQ ID NO: 6 is registered with the GenBank database under Accession No. NM_{—}017671, Accession No. NP_{—}060141, and represents human C20orf42 (URP1, also referred to as Kindlerin). The human C20orf42 was disclosed in Non-patent literature 9 (Biochim. Biophys. Acta 1637: 207-216 (2003)).

The protein of the present invention BUB1 comprises the amino acid sequence of SEQ ID NO: 8 or an amino acid sequence similar to the aforementioned amino acid sequence. The protein BUB1 of the invention may be a protein originated from natural source (e.g., a pancreas cancer cell line PUN) or a recombinant protein. Here, the amino acid sequence of SEQ ID NO: 8 is registered with the GenBank database under Accession No. NM_{—}004336, Accession No. NP_{—}004327, and represents human BUB1. The human BUB1 was disclosed in a literature (Genomics 46:379-388 (1997)).

The protein of the present invention C10orf3 comprises the amino acid sequence of SEQ ID NO: 10 or an amino acid sequence similar to the aforementioned amino acid sequence. The protein C10orf3 of the invention may be a protein originated from natural source (e.g., a lung adenocarcinoma cell line 1-87) or a recombinant protein. Here, the amino acid sequence of SEQ ID NO: 10 is registered with the GenBank database under Accession No. NM_{—}018131, Accession No. NP_{—}060601, and represents human C10orf3. The human C10orf3 was disclosed in Non-patent literature 16 (Proc. Natl. Acad. Sci. USA., December 24; 99(26):16899-903 (2002)).

The protein of the present invention HIFPH3 comprises the amino acid sequence of SEQ ID NO: 12 or an amino acid sequence similar to the aforementioned amino acid sequence. The protein HIFPH3 of the invention may be a protein originated from natural source (e.g., a renal cancer cell line SMKTR-1) or a recombinant protein. Here, the amino acid sequence of SEQ ID NO: 12 is registered with the GenBank database under Accession No. NM_{—}022073, Accession No. NP_{—}071356, and represents human HIFPH3 (egl nine homolog 3, also referred to as EGLN3). The human HIFPH3 was disclosed in Non-patent literature 17 (Cell 107: 43-54 (2001))

Each of Lengsin, BJ-TSA-9, C20orf42, BUB1, C10orf3, and HIFPH3 is also referred herein as “the protein of the invention”.

The “protein comprising the amino acid sequence of SEQ ID NO: 2, 4, 6, 8, 10 or 12” specifically includes a protein consisting of the amino acid sequence of SEQ ID NO: 2, 4, 6, 8, 10 or 12, and a protein consisting of an amino acid sequence which comprises the amino acid sequence of SEQ ID NO: 2, 4, 6, 8, 10 or 12 having an additional amino acid sequence(s) attached to the N and/or C terminus. “Additional amino acid sequence” may be the amino acid sequence derived from other structural genes than the proteins of the invention.

The “protein comprising an amino acid sequence similar to the amino acid sequence of SEQ ID NO: 2, 4, 6, 8, 10 or 12” specifically includes the following proteins (a) to (c):

(a) a protein comprising an amino acid sequence which is the same as the amino acid sequence of SEQ ID NO: 2, 4, 6, 8, 10 or 12 except that one or more amino acids are deleted, substituted and/or added, and having an activity as a tumor antigen protein;

(b) a protein comprising an amino acid sequence having at least 70% sequence identity with the amino acid sequence of SEQ ID NO: 2, 4, 6, 8, 10 or 12, and having an activity as a tumor antigen protein;

(c) a protein being encoded by a polynucleotide capable of hybridizing to a complementary strand of a polynucleotide encoding the amino acid sequence of SEQ ID NO: 2, 4, 6, 8, 10 or 12 under stringent conditions, and having an activity as a tumor antigen protein.

Preferred examples include a protein consisting of an amino acid sequence similar to the amino acid sequence of SEQ ID NO: 2, 4, 6, 8, 10 or 12. Examples of such a protein include the proteins (a′) to (c′) below:

(a′) a protein consisting of an amino acid sequence which is the same as the amino acid sequence of SEQ ID NO: 2, 4, 6, 8, 10 or 12 except that one or more amino acids are deleted, substituted and/or added, and having an activity as a tumor antigen protein;

(b′) a protein consisting of an amino acid sequence having at least 70% sequence identity with the amino acid sequence of SEQ ID NO: 2, 4, 6, 8, 10 or 12, and having an activity as a tumor antigen protein;

(c′) a protein being encoded by a polynucleotide capable of hybridizing to a complementary strand of a polynucleotide encoding the amino acid sequence of SEQ ID NO: 2, 4, 6, 8, 10 or 12 under stringent conditions, and having an activity as a tumor antigen protein.

The “protein comprising an amino acid sequence which is the same as the amino acid sequence of SEQ ID NO: 2, 4, 6, 8, 10 or 12 except that one or more amino acids are deleted, substituted and/or added” in (a) above refers to a protein produced artificially, that is, a modified (variant) protein, or an allele variant present in a living body, for example.

In this respect, there is no limitation regarding the number or position of modification (mutation) in the protein as far as the activity of the protein of the invention is maintained. Criteria based on which one can determine the number or position of the amino acid residue to be deleted, substituted and/or added without reducing the activity can be obtained using a computer program well known in the art, such as DNA Star software. For example, the number of mutation would typically be within 10%, preferably 5% of the total amino acid residues. Furthermore, the amino acid introduced by substitution preferably has similar characteristics to that to be substituted in view of retention of structure, which characteristics include polarity, charge, solubility, hydrophobicity, hydrophilicity, amphipathicity, and the like. For instance, Ala, Val, Leu, Ile, Pro, Met, Phe and Trp are classified into nonpolar amino acids; Gly, Ser, Thr, Cys, Tyr, Asn and Gln into non-charged amino acids; Asp and Glu into acidic amino acids; and Lys, Arg and His into basic amino acids. One of ordinary skill in the art can select an appropriate amino acid(s) falling within the same group on the basis of these criteria.

The “protein comprising an amino acid sequence having at least 70% sequence identity with the amino acid sequence of SEQ ID NO: 2, 4, 6, 8, 10 or 12” in (b) above includes a protein comprising an amino acid sequence having at least about 70%, preferably about 80%, more preferably about 90%, and further more preferably about 95% sequence identity with the amino acid sequence of SEQ ID NO: 2, 4, 6, 8, 10 or 12, and specifically, a protein consisting of a partial amino acid sequence of SEQ ID NO: 2, 4, 6, 8, 10 or 12.

The term “sequence identity” herein used refers to the identity and homology between two proteins. The “sequence identity” is determined by comparing two sequences aligned optimally over the sequence region to be compared. In this context, the optimum alignment of the proteins to be compared may have an addition or deletion (e.g., “gap”)

The sequence identity can be calculated by preparing an alignment using, for example, Vector NTI, ClustalW algorithm (Nucleic Acid Res., 22 (22): 4673-4680 (1994)). The sequence identity can be determined using software for sequence analysis, specifically, Vector NTI or GENETYX-MAC, or a sequencing tool provided by a public database. Such a public database is commonly available at Web site (http://www.ddbj.nig.ac.ip).

The “polynucleotide capable of hybridizing to a complementary strand of a polynucleotide encoding the amino acid sequence of SEQ ID NO: 2, 4, 6, 8, 10 or 12 under stringent conditions” in (c) above includes a polynucleotide comprising a base sequences having at least about 40%, preferably about 60%, more preferably about 70%, still more preferably about 80%, further more preferably about 90%, and most preferably about 95% sequence identity with a polynucleotide encoding the amino acid sequence of SEQ ID NO: 2, 4, 6, 8, 10 or 12. Specifically, a polynucleotide comprising a base sequence having at least about 40%, preferably about 60%, more preferably about 70%, still more preferably about 80%, further more preferably about 90%, and most preferably about 95% sequence identity with the base sequence of SEQ ID NO: 1, 3, 5, 7, 9 or 11 is exemplified. More specifically, a polynucleotide consisting of a partial sequence of the base sequence of SEQ ID NO: 1, 3, 5, 7, 9 or 11 is exemplified.

Hybridization can be conducted according to a method known per se or a method equivalent thereto, for example, that described in a fundamental text book such as “Molecular Cloning 2nd Edt. Cold Spring Harbor Laboratory Press (1989)”, and the like. Also, it can be performed using a commercially available library according to the instructions attached thereto.

The “stringent conditions” herein used can be determined on the basis of the melting temperature (Tm) of nucleic acids forming a complex or a nucleic acid binding to a probe as described in literatures (Berger and Kimmel, 1987, “Guide to Molecular Cloning Techniques Methods in Enzymology”, Vol. 152, Academic Press, San Diego Calif.; or “Molecular Cloning” 2nd Edt. Cold Spring Harbor Laboratory Press (1989), ibid.).

For example, hybridization can be carried out in a solution containing 6×SSC (20×SSC means 333 mM sodium citrate, 333 mM NaCl), 0.5% SDS and 50% formamide at 42° C., or in a solution containing 6×SSC (without 50% formamide) at 65° C.

Washing after the hybridization can be conducted under a condition around “1×SSC, 0.1% SDS, 37° C.”. The complementary strand preferably remains to be bound to the target forward strand when washed under such washing conditions. More stringent hybridization conditions may involve washing conditions of around “0.5×SSC, 0.1% SDS, 42° C.” and still more stringent hybridization conditions around “0.1×SSC, 0.1% SDS, 65° C.”, although it is not limited thereto.

The protein of the invention has an activity as a tumor antigen protein. The term “activity as a tumor antigen protein” refers to an activity detected by a conventional assay for the activity of a tumor antigen protein. Specifically, it refers to the characteristics that a cell expressing Lengsin, BJ-TSA-9, C20orf42, BUB1, C10orf3 or HIFPH3 is recognized by a CTL, that is, the cell exhibits reactivity to a CTL, in other words, the protein of the present invention or antigen peptides derived therefrom activates or induces a CTL.

In this respect, the “cell” preferably expresses an HLA antigen. Accordingly, the “activity as a tumor antigen protein” more specifically refers to the characteristics that, when the protein of the present invention is expressed in a cell expressing an HLA antigen such as HLA-A24 or HLA-A2, a complex between a tumor antigen peptide originated from the protein of the invention and the HLA antigen is presented on the cell surface and consequently the cell is recognized by a CTL, in other words, a CTL is activated (induced).

The characteristics of the protein of the invention as mentioned above can be easily determined by a known method or a method equivalent thereto, such as ⁵¹Cr release assay (J. Immunol., 159: 4753, 1997), LDH release assay using LDH Cytotoxicity Detection Kit (Takara Bio, Inc.), measurement of cytokines, and the like. The detailed protocol of assay will hereinafter be illustrated.

First, a host cell such as 293-EBNA cell (Invitrogen) is co-transfected with an expression vector comprising a DNA encoding the protein of the invention and an expression vector comprising a DNA encoding an HLA antigen. The DNA encoding an HLA antigen includes a DNA encoding HLA-A24 antigen or HLA-A2 antigen. Examples of a DNA encoding HLA-A24 antigen include HLA-A2402 cDNA (Cancer Res., 55: 4248-4252 (1995), Genbank Accession No. M64740). Examples of DNA encoding HLA-A2 antigen include HLA-A0201 cDNA (GenBank Acc. No. M84379).

The transfection as mentioned above can be conducted by Lipofectin method using lipofectamine reagent (GIBCO BRL), and the like. Then, a CTL restricted to the HLA antigen used is added and allowed to react, followed by measurement of various cytokines (for example, IFN-γ) produced by the activated (reacting) CTL by a method such as ELISA, for example. The CTL usable herein may be prepared by stimulating peripheral blood lymphocytes with the protein of the invention or established according to the method of Int. J. Cancer, 39, 390-396, 1987, N. Eng. J. Med, 333, 1038-1044, 1995, or the like.

The CTL-inducing activity of the protein of the invention can also be examined in vivo by an assay where a model animal for human is used (WO 02/47474; Int. J. Cancer. 100, 565-570 (2002)).

The protein of the present invention can be prepared by a method known per se that is used for purifying a protein from natural products (e.g., cancer cell lines) or by a method hereinafter described comprising culturing a transformant carrying a nucleic acid comprising a polynucleotide encoding the protein of the present invention.

2) Peptide Derived from the Protein of the Invention

The peptide of the present invention, which may be referred to as “the peptide of the invention” or “the tumor antigen peptide of the present invention”, is a tumor antigen peptide which comprises a partial peptide of Lengsin, BJ-TSA-9, C20orf42, BUB1, C10orf3 or HIFPH3 and is capable of binding to an HLA antigen and is recognized by a CTL. Thus, the peptide of the present invention may comprise a peptide corresponding to any position of the amino acid sequence of the protein of the invention and being of any length, as long as the peptide comprises a part of the amino acid sequence of the protein of the invention as defined above and can form a complex with an HLA antigen that is recognized by a CTL.

The peptide of the invention can be identified by synthesizing a candidate peptide, which is a partial fragment of Lengsin, BJ-TSA-9, C20orf42, BUB1, C10orf3 or HIFPH3, and subjecting the candidate peptide to an assay to examine whether or not a CTL recognizes a complex between the candidate peptide and an HLA antigen, that is, whether or not the candidate peptide has the activity as a tumor antigen peptide.

Synthesis of the peptide can be conducted according to a method generally used in the field of peptide chemistry. Such a method can be found in literatures including Peptide Synthesis, Interscience, New York, 1966; The Proteins, Vol. 2, Academic Press Inc., New York, 1976; Peptide-Gosei, Maruzen, Inc., 1975; Peptide-Gosei no Kiso to Jikken, Maruzen, Inc., 1985; and Iyakuhin no Kaihatsu (Zoku), Vol. 14, Peptide-Gosei, Hirokawa-syoten, 1991.

The method for identification of the tumor antigen peptide of the present invention will hereinafter be described in detail.

The regularity (motif) in an amino acid sequence of a tumor antigen peptide that binds to an HLA antigen to be presented has been elucidated in relation to some HLA types such as HLA-A1, -A0201, -A0204, -A0205, -A0206, -A0207, -A11, -A24, -A31, -A6801, -B7, -B8, -B2705, -B37, -Cw0401, and -Cw0602. See, Immunogenetics, 41: p. 178, 1995, etc. For example, the motifs for HLA-A24 are known to have an amino acid sequence of 8 to 11 amino acids, wherein the amino acid at position 2 is tyrosine, phenylalanine, methionine or tryptophan, and the C-terminal amino acid phenylalanine, leucine, isoleucine, tryptophan or methionine (J. Immunol., 152, p 3913, 1994, Immunogenetics, 41: p 178, 1995. J. Immunol., 155:p 4307, 1994). As for motifs for HLA-A2, those listed in Table 1 are known (Immunogenetics, 41, p 178, 1995, J. Immunol., 155: p 4749, 1995).

TABLE 1

| | Amino acid | |
|---|---|---|
| | at position 2 | Amino acid |
| HLA-A2 type | from N-terminus | at C-terminus |
| HLA-A0201 | L, M | V, L |
| HLA-A0204 | L | L |
| HLA-A0205 | V, L, I, M | L |
| HLA-A0206 | V, Q | V, L |
| HLA-A0207 | L | L |

* All the peptides are 8 to 11 amino acids in length.

Recently, it has become possible to search a peptide sequence expected to be capable of binding to an HLA antigen via the internet using BIMAS software; NIH (http://bimas.dcrt.nih.gov/molbio/hla_bind/).

As for the length of the peptide, analysis of antigen peptides binding to various HLA molecules revealed that it is generally about 8 to 14 amino acids (Immunogenetics, 41: 178, 1995). However, in the cases of HLA-DR, -DP, and -DQ, peptides consisting of 14 amino acids or more are also known.

It is easy to select a portion corresponding to the peptide from the amino acid sequence of the protein of the invention considering the motif. For example, a sequence expected to be capable of binding to an HLA antigen may be easily selected by means of BIMAS software. The peptide of the present invention can be identified by synthesizing the selected candidate peptide by the above-mentioned method, and examining whether or not the candidate peptide binds to an HLA antigen and is recognized by a CTL, that is, whether or not the candidate peptide has an activity as a tumor antigen peptide.

Specifically, identification can be done by the method descried in J. Immunol., 154, p 2257, 1995. Thus, a candidate peptide is added to stimulate in vitro peripheral blood lymphocytes isolated from a human subject positive for an HLA antigen which is expected to present the candidate peptide. When a CTL specifically recognizing the HLA-positive cell pulsed with the candidate peptide is induced, the candidate peptide is possibly a tumor antigen peptide. Whether or not the induction of CTL occurs may be examined by, for example, measuring the amount of various cytokines (e.g., IFN-γ) produced by the CTL in response to the antigen-presenting cell using ELISA or the like. Alternatively, the induction of CTL can also be examined by ⁵¹Cr release assay wherein the cytotoxicity of a CTL against an antigen-presenting cell labeled with ⁵¹Cr is measured (Int. J. Cancer, 58: p 317, 1994). Furthermore, the induction of CTL can be examined by pulsing a cell such as 293-EBNA cell (Invitrogen) with a candidate peptide, wherein the cell has been introduced with an expression plasmid for cDNA encoding a type of HLA antigens expected to present the candidate peptide, reacting the cell with a CTL restricted to the HLA antigen of the aforementioned type that is expected to present the candidate peptide, and measuring various cytokines (e.g., IFN-γ) produced by the CTL (J. Exp. Med., 187: 277, 1998).

Examples of the HLA antigen include HLA-A24 antigen and HLA-A2 antigen. To select an HLA-A24-restricted tumor antigen peptide, HLA-A2402 cDNA (Cancer Res., 55: 4248-4252 (1995), Genbank Accession No. M64740) can be used as the cDNA encoding the HLA antigen. To select an HLA-A2-restricted tumor antigen peptide, HLA-A0201 cDNA (GenBank Acc. No. M84379) can be used as the cDNA encoding the HLA antigen.

As for CTLs, in addition to those obtained by stimulating human peripheral blood lymphocytes with a peptide, CTLs established by a method described in literatures (Int. J. Cancer, 39, 390-396, 1987; N. Eng. J. Med, 333, 1038-1044, 1995) may be used.

The in vivo activity of the peptide of the present invention can be determined by an assay which uses an animal model for human (WO 02/47474, Int J. Cancer 100, 565-570 (2002)).

In the above case, the regularity (motif) of the sequence of a tumor antigen peptide is known; however, when the motif of a peptide is unknown, as is the case for HLA-B55 or HLA-A26, the tumor antigen peptide of the present invention can be identified according to the method described in, for example, WO97/46676, only if a CTL cell line capable of recognizing a complex between the HLA antigen and a tumor antigen peptide is available.

Specific examples of the peptide of the present invention include a partial peptide derived from Lengsin consisting of the amino acid sequence of SEQ ID NO: 2, BJ-TSA-9 consisting of the amino acid sequence of SEQ ID NO: 4, C20orf42 consisting of the amino acid sequence of SEQ ID NO: 6, BUB1 consisting of the amino acid sequence of SEQ ID NO: 8, C10orf3 consisting of the amino acid sequence of SEQ ID NO: 10, or HIFPH3 consisting of the amino acid sequence of SEQ ID NO: 12, and being capable of binding to an HLA antigen and being recognized by a CTL. Preferred examples include a peptide capable of binding to HLA-A24 or HLA-A2 antigen, considering the HLA antigen to which the peptide of the present invention binds. The length of the peptide may be preferably 8 to 14 amino acids, more preferably 8 to 11 amino acids.

Specifically, the peptide of the present invention includes a peptide comprising the amino acid sequence of any one of SEQ ID NOS: 13 to 201 and being capable of binding to an HLA antigen and being recognized by a CTL. The length of the peptide may be preferably 9 to 14 amino acids, more preferably 9 to 11 amino acids. More specifically, as an HLA-A24-binding tumor antigen peptide, a peptide consisting of any one of the amino acid sequences of SEQ ID NOS: 13 to 31, 42 to 49, 59 to 78, 89 to 117, 158 to 165, 176 to 183 and 195 to 201, being capable of binding to HLA-A24 antigen and being recognized by a CTL (see Tables 5, 7, 9, 11, 13 and 15 below) is exemplified. Preferably, a peptide consisting of the amino acid sequence of SEQ ID NO: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 or 183 is exemplified.

More preferably, a peptide consisting of the amino acid sequence of SEQ ID NO: 158, 22, 23, 26, 27, 198, 200, 201, 177, 178, 179 or 183 is exemplified.

Further, as an HLA-A2-binding tumor antigen peptide, a peptide consisting of any one of the amino acid sequences of SEQ ID NOS: 32 to 41, 50 to 58, 79 to 88, 118 to 157, 166 to 175 and 184 to 194, being capable of binding to HLA-A2 antigen and being recognized by a CTL is exemplified (see Tables 6, 8, 10, 12, 14 and 16 below).

In the scope of the present invention, the peptide of the present invention includes not only a peptide consisting of a part of the amino acid sequence of SEQ ID NO: 2, 4, 6, 8, 10 or 12 but also a variant (modified) peptide produced by partly modifying the aforementioned peptide, provided that the variant peptide has characteristics of being capable of binding to an HLA antigen and being recognized by a CTL. Specifically, a variant peptide comprising an amino acid sequence which is the same as the amino acid sequence of the peptide of the present invention consisting of a part of the amino acid sequence of Lengsin, BJ-TSA-9, C20orf42, BUB1, C10orf3 or HIFPH3, specifically the amino acid sequence of SEQ ID NO: 2, 4, 6, 8, 10 or 12, except that at least one amino acid modification has been introduced, and having an activity as a tumor antigen peptide, i.e. being capable of binding to an HLA antigen and being recognized by a CTL, falls within the scope of the present invention.

The “modification” of an amino acid residue means substitution, deletion and/or addition of an amino acid residue including addition to the N- and/or C-terminus of peptide, and is preferably substitution of an amino acid residue. When the modification involves substitution of an amino acid residue, the number or position of the amino acid residue(s) to be substituted can be selected arbitrarily as far as an activity as a tumor antigen peptide is maintained; however, it is preferred that the substitution involves 1 to several amino acid residues since tumor antigen peptides are generally about 8-14 amino acids in length as mentioned above.

The variant peptide of the present invention is preferably 8 to 14 amino acids in length (in the cases of HLA-DR, -DP, or -DQ, however, peptides consisting of 14 amino acids or more are acceptable).

As mentioned above, the motif in an antigen peptide that binds to an HLA antigen and is presented is known in regard to certain HLA types, such as HLA-A1, -A0201, -A0204, -A0205, -A0206, -A0207, -A11, -A24, -A31, -A6801, -B7, -B8, -B2705, -B37, -Cw0401 and -Cw0602. Further, it is possible to search for a peptide sequence expected to be able to bind to an HLA antigen via internet (http://bimas.dcrt.nih.gov/molbio/hla_bind/). Thus, one can prepare the variant peptide above on the basis of the motif and the like.

For example, as hereinbefore described, the motif of an antigen peptide being capable of binding to HLA-A24 and being presented is known as a sequence characterized in that, in an 8 to 11 amino acids peptide, the amino acid at position 2 is tyrosine, phenylalanine, methionine or tryptophan, and the C terminal amino acid is phenylalanine, leucine, isoleucine, tryptophan or methionine (J. Immunol., 152: p 3913, 1994; Immunogenetics, 41: p 178, 1995; J. Immunol., 155: p 4307, 1994). As for HLA-A2, the motif is known as a sequence characterized in that, in a 8 to 11 amino acids peptide, the amino acid at position 2 is leucine, methionine, valine, isoleucine or glutamine and the C terminal amino acid is valine or leucine (Immunogenetics, 41: p 178, 1995; J. Immunol., 155: p 4749, 1995). Furthermore, some peptide sequences that are expected to be able to bind to an HLA antigen are published via internet (httt://bimas.dcrt.nih.gov/molbio/hla bind/). Amino acids having similar characteristics to those available for the motif above are also acceptable. Thus, the present invention includes a variant peptide comprising an amino acid sequence which is the same as the amino acid sequence of the peptide of the present invention except that an amino acid(s) at position(s) available for substitution in light of the motif (in the case of HLA-A24 and HLA-A2, position 2 and C-terminus) is substituted by another amino acid, preferably by an amino acid expected to provide a binding activity from the internet site as mentioned above, for example, and having an activity of binding to the HLA antigen and being recognized by a CTL.

More preferably, the present invention includes a variant peptide whose amino acid residue(s) at the aforementioned position(s) is substituted by another amino acid known to be available in light of the motif and having an activity as a tumor antigen peptite. Thus, in the case of HLA-A24-binding peptides as shown in SEQ ID NOS: 13 to 31, 42 to 49, 59 to 78, 89 to 117, 158 to 165, 176 to 183 and 195 to 201, examples of variant peptides include those comprising an amino acid sequence which is the same as the amino acid sequence of any one of SEQ ID NOS: 13 to 31, 42 to 49, 59 to 78, 89 to 117, 158 to 165, 176 to 183 and 195 to 201 except that the amino acid at position 2 is substituted by tyrosine, phenylalanine, methionine or tryptophan, and/or the C terminal amino acid by phenylalanine, leucine, isoleucine, tryptophan or methionine, and being capable of binding to HLA-A24 antigen and being recognized by a CTL. Above all, a peptide whose amino acid at position 2 is substituted by tyrosine is more preferred.

More preferably, the variant peptide consists of an amino acid sequence which is the same as the amino acid sequence of any one of SEQ ID NOS: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 and 183 except that the amino acid at position 2 is substituted by tyrosine, phenylalanine, methionine or tryptophan, and/or the C terminal amino acid by phenylalanine, leucine, isoleucine, tryptophan or methionine, and is capable of binding to HLA-A24 antigen and is recognized by a CTL. More preferably, the variant peptide consists of an amino acid sequence which is the same as the amino acid sequence of any one of SEQ ID NOS: 158, 22, 23, 26, 27, 198, 200, 201, 177, 178, 179 and 183 except that the amino acid at position 2 and/or the C terminal amino acid are substituted as mentioned above, and is capable of binding to HLA-A24 antigen and is recognized by a CTL.

In the case of HLA-A2-binding peptides as shown in SEQ ID NOS: 32 to 41, 50 to 58, 79 to 88, 118 to 157, 166 to 175 and 184 to 194, a preferable variant peptide is that comprising an amino acid sequence which is the same as the amino acid sequence of any one of SEQ ID NOS: 32 to 41, 50 to 58, 79 to 88, 118 to 157, 166 to 175 and 184 to 194 except that the amino acid at position 2 is substituted by leucine, methionine, valine, isoleucine or glutamine and/or the C terminal amino acid by valine or leucine, and being capable of binding to HLA-A2 antigen and being recognized by a CTL.

The peptide of the present invention further includes an epitope peptide comprising the tumor antigen peptide of the present invention as mentioned above.

Recently, a peptide composed of multiple (plural) CTL epitopes (antigen peptides) ligated together (“epitope peptide”) has been shown to induce CTLs efficiently. For example, it has been reported that a peptide (about 30-mer) composed of CTL epitopes originated from a tumor antigen protein PSA each restricted to HLA-A2-, -A3, -A11, or B53 ligated together induced in vivo CTLs specific for respective CTL epitopes (Journal of Immunology 1998, 161: 3186-3194).

In addition, a peptide (epitope peptide) composed of a CTL epitope and a helper epitope ligated together has been shown to induce a CTL efficiently. In this context, “helper epitope” means a peptide capable of activating CD4-positive T cells (Immunity., 1:751, 1994), and examples thereof include HBVc128-140 of hepatitis B virus origin, TT947-967 of tetanus toxin origin, and the like. CD4⁺ T cells activated with the helper epitope exert some activities including induction and maintenance of CTLs, and activation of effectors such as macrophages, and hence are considered to be important in the immunological anti-tumor response. As a specific example of the peptide composed of a helper epitope and a CTL epitope ligated together, it is reported that a DNA (minigene) composed of six kinds of HBV-derived HLA-A2-restricted antigen peptides, three kinds of HLA-A11-restricted antigen peptides and a helper epitope induced in vivo CTLs directed to the respective epitopes efficiently (Journal of Immunology 1999, 162: 3915-3925). Practically, a peptide composed of a CTL epitope (a tumor antigen peptide corresponding to position 280-288 of melanoma antigen gp100) and a helper epitope (tetanus toxin-derived T helper epitope) ligated has been subjected to clinical test (Clinical Cancer Res., 2001, 7:3012-3024).

Accordingly, the tumor antigen peptide of the present invention also includes a peptide (epitope peptide) composed of multiple epitopes including the peptide of the present invention ligated together and having an activity of inducing a CTL.

In this respect, the “epitope peptide” is defined as (1) a peptide composed of two or more CTL epitopes (tumor antigen peptides) ligated together, or (2) a peptide composed of a CTL epitope(s) and a helper epitope(s) ligated together, which is processed in an antigen-presenting cell(s) to give a tumor antigen peptide(s) then being presented by the cell(s) and induces a CTL(s).

When a CTL epitope is ligated to the peptide of the present invention, the CTL epitope may be that derived from the amino acid sequence of Lengsin as shown in SEQ ID NO: 2, BJ-TSA-9 as shown in SEQ ID NO: 4, C20orf42 as shown in SEQ ID NO: 6, BUB1 as shown in SEQ ID NO: 8, C10orf3 as shown in SEQ ID NO: 10 or HIFPH3 as shown in SEQ ID NO: 12 and being restricted to HLA-A1, -A0201, -A0204, -A0205, -A0206, -A0207, -A11, -A24, -A31, -A6801, -B7, -B8, -B2705, -B37, -B55, -Cw0401, -Cw0602, and the like. CTL epitopes derived from other tumor antigen proteins are also usable. Plural number of CTL epitopes can be ligated together, and the length of a CTL epitope may be about 8-14 amino acids based on the analysis of antigen peptides binding to various HLA molecules (Immunogenetics, 41: 178, 1995).

When the a helper epitope is ligated to the peptide of the present invention, the helper epitope may be the aforementioned HBVc128-140 of hepatitis B virus origin, TT947-967 of tetanus toxin origin, and the like. The helper epitope may be about 13-30 amino acids, preferably about 13-17 amino acids in length.

The peptide (epitope peptide) composed of multiple epitopes ligated together can be prepared by the aforementioned conventional method for peptide synthesis. It can also be prepared by a conventional method for DNA synthesis and genetic engineering on the basis of the sequence information of a polynucleotide encoding an epitope peptide composed of multiple epitopes ligated together. That is, an epitope peptide composed of multiple epitopes ligated together can be prepared by inserting a polynucleotide encoding the epitope peptide into a known expression vector, transforming a host cell with the resultant recombinant expression vector, culturing the transformant, and recovering the desired epitope peptide from the culture. These processes can be conducted according to, for example, a method described in literatures (Molecular Cloning, T. Maniatis et al., CSH Laboratory (1983), DNA Cloning, D M. Glover, IRL PRESS (1985)).

The epitope peptide produced as mentioned above, which is composed of multiple epitopes ligated together, can be examined for CTL-inducing activity in vitro by means of an assay as mentioned above, or in vivo by means of an assay described in WO02/47474 or Int J. Cancer. 100, 565-570 (2002) using a model animal for human.

Also, the amino group of the N-terminal amino acid or the carboxyl group of the C-terminal amino acid of the tumor antigen peptide of the present invention can be modified. The peptide undergone such modification also falls within the scope of the present invention.

The modification of the amino group of the N-terminal amino acid involves 1 to 3 groups selected from C₁₋₆ alkyl group, phenyl group, cycloalkyl group and acyl group, for example. The acyl group specifically includes C₁₋₆ alkanoyl group, C₁₋₆ alkanoyl group substituted by phenyl group, carbonyl group substituted by C₅₋₇ cycloalkyl group, C₁₋₆ alkylsulfonyl group, phenylsulfonyl group, C₂₋₆ alkoxycarbonyl group, alkoxycarbonyl group substituted by phenyl group, carbonyl group substituted by C₅₋₇ cycloalkoxy group, phenoxycarbonyl group, and the like.

The peptide modified at the carboxyl group of the C-terminal amino acid may be ester or amide form. The ester specifically includes C₁₋₆ alkyl ester, C₀₋₆ alkyl ester substituted by phenyl group, C₅₋₇ cycloalkyl ester, and the like. The amide specifically includes amide, amide substituted by one or two C₁₋₆ alkyl groups, amide substituted by one or two C₀₋₆ alkyl groups wherein the alkyl group is substituted by phenyl group, amide forming 5- to 7-membered azacycloalkane including nitrogen atom of amide group, and the like.

### 3) Nucleic Acid of the Present Invention

The nucleic acid of the present invention specifically refers to

(1) a nucleic acid comprising the polynucleotide encoding Lengsin, BJ-TSA-9, C20orf42, BUB1, C10orf3 or HIFPH3, and
(2) a nucleic acid comprising the polynucleotide encoding the peptide of the present invention.
(1) Polynucleotide Encoding Lengsin, BJ-TSA-9, C20orf42, BUB1, C10orf3 or HIFPH3 and Nucleic Acid Comprising the Same

The polynucleotide encoding Lengsin, BJ-TSA-9, C20orf42, BUB1, C10orf3 or HIFPH3 can be cDNA or mRNA, cRNA or genomic DNA of various cells or tissues such as those originated from prostate cancer, or synthetic DNA. It may be either single- or double-stranded. Specifically, the polynucleotide includes the followings:

(a) a polynucleotide comprising the base sequence of SEQ ID NO: 1, 3, 5, 7, 9 or 11;
(b) a polynucleotide comprising a base sequence encoding the amino acid sequence of SEQ ID NO: 2, 4, 6, 8, 10 or 12; and
a polynucleotide comprising a base sequence similar to that of the polynucleotide (a) or (b).

In this respect, the base sequence of SEQ ID NO: 1 corresponds to the base sequence of human Lengsin gene which is registered with GenBank database under Accession No. NM_{—}016571. The amino acid sequence of SEQ ID NO: 2 corresponds to that of human Lengsin which is registered with GenBank database under Accession No. NM_{—}016571, Accession No. NP_{—}057655. The human Lengsin gene was disclosed in Non-patent literature 7 (Mol. Vis. Jun. 15; 8:185-95 (2002)).

The base sequence of SEQ ID NO: 3 corresponds to the base sequence of human BJ-TSA-9 gene which is registered with GenBank database under Accession No. NM_{—}032899. The amino acid sequence of SEQ ID NO: 4 corresponds to that of human BJ-TSA-9 which is registered with GenBank database under Accession No. NM_{—}032899, Accession No. NP_{—}116288. The human BJ-TSA-9 gene was disclosed in Non-patent literature 8 (Proc. Natl. Acad. Sci. USA., 99(26):16899-903 (2002)).

The base sequence of SEQ ID NO: 5 corresponds to the base sequence of human C20orf42 gene which is registered with GenBank database under Accession No. NM_{—}017671. The amino acid sequence of SEQ ID NO: 6 corresponds to that of human C20orf42 which is registered with GenBank database under Accession No. NM_{—}017671, Accession No. NP_{—}060141. The human C20orf42 gene was disclosed in Non-patent literature 9 (Biochim. Biophys. Acta 1637: 207-216 (2003)).

The base sequence of SEQ ID NO: 7 corresponds to the base sequence of human BUB1 gene which is registered with GenBank database under Accession No. NM_{—}004336. The amino acid sequence of SEQ ID NO: 8 corresponds to that of human BUB1 which is registered with GenBank database under Accession No. NM_{—}004336, Accession No. NP_{—}004327. The human BUB1 gene was disclosed in a literature (Genomics 46:379-388 (1997)).

The base sequence of SEQ ID NO: 9 corresponds to the base sequence of human C10orf3 gene which is registered with GenBank database under Accession No. NM_{—}018131. The amino acid sequence of SEQ ID NO: 10 corresponds to that of human C10orf3 which is registered with GenBank database under Accession No. NM_{—}018131, Accession No. NP_{—}060601. The human C10orf3 gene was disclosed in Non-patent literature 16 (Proc. Natl. Acad. Sci. USA., December 24; 99(26):16899-903 (2002)).

The base sequence of SEQ ID NO: 11 corresponds to the base sequence of human HIFPH3 gene which is registered with GenBank database under Accession No. NM_{—}022073. The amino acid sequence of SEQ ID NO: 12 corresponds to that of human HIFPH3 which is registered with GenBank database under Accession No. NM_{—}022073, Accession No. NP_{—}071356. The human HIFPH3 gene was disclosed in Non-patent literature 17 (Cell 107: 43-54 (2001)).

The aforementioned (a) polynucleotide comprising the base sequence of SEQ ID NO: 1, 3, 5, 7, 9 or 11 and (b) polynucleotide comprising a base sequence encoding the amino acid sequence of SEQ ID NO: 2, 4, 6, 8, 10 or 12 specifically include a polynucleotide consisting of the base sequence of SEQ ID NO: 1, 3, 5, 7, 9 or 11 and a polynucleotide consisting of a base sequence encoding the amino acid sequence of SEQ ID NO: 2, 4, 6, 8, 10 or 12. Further example includes a polynucleotide consisting of a base sequence which contains the base sequence of SEQ ID NO: 1, 3, 5, 7, 9 or 11 or that encoding the amino acid sequence of SEQ ID NO: 2, 4, 6, 8, 10 or 12, to which an additional base sequence is added at the 5′- and/or 3′-terminus. “Additional base sequence” may be a base sequence encoding a structural gene other than Lengsin, BJ-TSA-9, C20orf42, BUB1, C10orf3 and HIFPH3.

Such a polynucleotide encoding Lengsin, BJ-TSA-9, C20orf42, BUB1, C10orf3 or HIFPH3 is characterized in that the protein encoded by the polynucleotide has an activity as a tumor antigen protein. The activity and methods of determining the same are described in “1) The Protein of the Present Invention”.

A polynucleotide comprising the base sequence of SEQ ID NO: 1 can be cloned by screening a cDNA library derived from, for example, a lung adenocarcinoma cell line (such as 1-87) using an appropriate portion of the base sequence disclosed in GenBank Accession No. NM_{—}016571 or herein disclosed in SEQ ID NO: 1 as a probe for hybridization or a primer for PCR.

A polynucleotide comprising the base sequence of SEQ ID NO: 3 can be cloned by screening a cDNA library derived from, for example, a lung adenocarcinoma cell line (such as 1-87) using an appropriate portion of the base sequence disclosed in GenBank Accession No. NM_{—}032899 or herein disclosed in SEQ ID NO: 3 as a probe for hybridization or a primer for PCR.

A polynucleotide comprising the base sequence of SEQ ID NO: 5 can be cloned by screening a cDNA library derived from, for example, a colon cancer cell line (such as SW480 (ATCC Number: CCL-228)) using an appropriate portion of the base sequence disclosed in GenBank Accession No. NM_{—}017671 or herein disclosed in SEQ ID NO: 5 as a probe for hybridization or a primer for PCR.

A polynucleotide comprising the base sequence of SEQ ID NO: 7 can be cloned by screening a cDNA library derived from, for example, a pancreas cancer cell line (such as PUN) using an appropriate portion of the base sequence disclosed in GenBank Accession No. NM_{—}004336 or herein disclosed in SEQ ID NO: 7 as a probe for hybridization or a primer for PCR.

A polynucleotide comprising the base sequence of SEQ ID NO: 9 can be cloned by screening a cDNA library derived from, for example, a lung adenocarcinoma cell line (such as 1-87) using an appropriate portion of the base sequence disclosed in GenBank Accession No. NM_{—}018131 or herein disclosed in SEQ ID NO: 9 as a probe for hybridization or a primer for PCR.

A polynucleotide comprising the base sequence of SEQ ID NO: 11 can be cloned by screening a cDNA library derived from, for example, a renal cancer cell line (such as SMKTR-1) using an appropriate portion of the base sequence disclosed in GenBank Accession No. NM_{—}022073 or herein disclosed in SEQ ID NO: 11 as a probe for hybridization or a primer for PCR.

One ordinary skilled in the art can easily conduct the cloning according to the method described in Molecular Cloning 2nd Edt. Cold Spring Harbor Laboratory Press (1989), etc.

A polynucleotide comprising a base sequence similar to that of the polynucleotide (a) or (b) above specifically includes the followings:

(c) a polynucleotide capable of hybridizing to a complementary strand of the polynucleotide (a) or (b) under stringent conditions, which encodes a protein having an activity as a tumor antigen protein;

(d) a polynucleotide comprising a base sequence having at least 70% sequence identity with the polynucleotide (a) or (b), which encodes a protein having an activity as a tumor antigen protein; and

(e) a polynucleotide encoding a protein comprising an amino acid sequence which is the same as the amino acid sequence encoded by the polynucleotide (a) or (b) except that one or more amino acids are deleted, substituted and/or added, wherein the protein has an activity as a tumor antigen protein.

Preferred examples include a polynucleotide consisting of a base sequence similar to that of the polynucleotide (a) or (b) above. The polynucleotide consisting of a base sequence similar to that of the polynucleotide (a) or (b) above includes the polynucleotides (c′) to (e′) below: (c′) a polynucleotide capable of hybridizing to a complementary strand of the polynucleotide (a) or (b) under stringent conditions, which encodes a protein having an activity as a tumor antigen protein;

(d′) a polynucleotide consisting of a base sequence having at least 70% sequence identity with the polynucleotide (a) or (b), which encodes a protein having an activity as a tumor antigen protein; and

(e′) a polynucleotide encoding a protein consisting of an amino acid sequence which is the same as the amino acid sequence encoded by the polynucleotide (a) or (b) except that one or more amino acids are deleted, substituted and/or added, wherein the protein has an activity as a tumor antigen protein.

Examples of the “polynucleotide capable of hybridizing to a complementary strand of the polynucleotide (a) or (b) above under stringent conditions” include a polynucleotide comprising a base sequence having at least about 40%, preferably about 60%, more preferably about 70%, still more preferably about 80%, further more preferably about 90%, and most preferably about 95% sequence identity with the base sequence of the polynucleotide (a) or (b) above, and specifically, a polynucleotide consisting of a partial sequence of the polynucleotide (a) or (b) above.

Hybridization can be conducted according to a method known per se or a method equivalent thereto, for example, a method described in a fundamental text “Molecular Cloning 2nd Edt. Cold Spring Harbor Laboratory Press (1989)”, and the like. Also, it can be performed using a commercially available library according to the instructions attached thereto.

The “stringent conditions” herein used can be determined on the basis of the melting temperature (Tm) of nucleic acids forming a complex or a nucleic acid binding to a probe as described in literatures (Berger and Kimmel, 1987, “Guide to Molecular Cloning Techniques Methods in Enzymology”, Vol. 152, Academic Press, San Diego Calif.; or “Molecular Cloning” 2nd Edt. Cold Spring Harbor Laboratory Press (1989)).

For example, hybridization can be carried out in a solution containing 6×SSC (20×SSC corresponds to 333 mM sodium citrate, 333 mM NaCl), 0.5% SDS and 50% formamide at 42° C., or in a solution containing 6×SSC (without 50% formamide) at 65° C.

Washing after the hybridization can be conducted under a condition around “1×SSC, 0.1% SDS, 37° C.”. The complementary strand preferably remains to bind to the target forward strand when washed under such washing conditions. More stringent hybridization conditions may involve washing under the conditions of around “0.5×SSC, 0.1% SDS, 42° C.” and still more stringent hybridization conditions around “0.1×SSC, 0.1% SDS, 65° C.”, although it is not limited thereto.

The “polynucleotide comprising a base sequence having at least 70% sequence identity with the polynucleotide of (a) or (b) above” includes a polynucleotide comprising a base sequence having at least about 70%, preferably about 80%, more preferably about 90%, and most preferably about 95% sequence identity with the base sequence of the polynucleotide of (a) or (b) above, and specifically, a polynucleotide consisting of a partial sequence of the polynucleotide of (a) or (b) above.

The term “sequence identity” herein used refers to identity or homology between two polynucleotides. The “sequence identity” is determined by comparing two sequences by aligning them optimally over the region corresponding to the sequence to be compared. In this context, The optimum alignment of the two polynucleotides to be compared may have an addition or deletion (e.g., “gap”). Such sequence identity can be calculated by preparing alignment using, for example, Vector NTI, ClustalW algorithm (Nucleic Acid Res., 22 (22): 4673-4680 (1994)). The sequence identity can be determined using software for sequence analysis, specifically, Vector NTI or GENETYX-MAC, or a sequencing tool provided by a public database. Such a public database is commonly available at Web site (http://www.ddbj.nig.ac.ip).

A polynucleotide having such sequence identity can be prepared according to the aforementioned hybridization method, or conventional PCR reaction or a reaction for modifying a polynucleotide (deletion, addition or substitution) hereinafter described.

The “polynucleotide encoding a protein comprising an amino acid sequence which is the same as the amino acid sequence of the protein encoded by the polynucleotide (a) or (b) above except that one or more amino acids are deleted, substituted and/or added” includes a nucleic acid encoding a variant protein produced artificially or an allele variant present in a living body.

In this respect, there is no limitation regarding the number or position of amino acid modification (mutation) as far as the activity of the protein of the invention is maintained. Criteria based on which one can determine the number or position of the amino acid residue to be deleted, substituted and/or added without reducing the activity can be obtained using a computer program well known in the art, such as DNA Star software. For example, the number of mutation would typically be within 10%, preferably 5% of the total amino acid residues. Furthermore, the amino acid introduced by substitution preferably has similar characteristics such as polarity, charge, solubility, hydrophobicity, hydrophilicity, amphipathicity, and the like, to that to be removed in view of retention of structure. For instance, Ala, Val, Leu, Ile, Pro, Met, Phe and Trp are classified into nonpolar amino acids; Gly, Ser, Thr, Cys, Tyr, Asn and Gln into non-charged amino acids; Asp and Glu into acidic amino acids; and Lys, Arg and His into basic amino acids. One of ordinary skill in the art can select an appropriate amino acid(s) within the same group on the basis of these criteria.

The polynucleotide encoding such a variant protein may be prepared by various methods such as site-directed mutagenesis and PCR technique described in Molecular Cloning 2nd Edt., Cold Spring Harbor Laboratory Press (1989). It also can be prepared by a known method such as Gapped duplex or Kunkel method using a commercially available kit.

The polynucleotide encoding the protein of the invention as mentioned above encodes a protein having an activity as a tumor antigen protein. “Having an activity as a tumor antigen protein” means that the protein is positive in a conventional assay for the activity of a tumor antigen protein. Specifically, it refers to the characteristics that a cell expressing the polynucleotide encoding the protein of the invention is recognized by a CTL, that is, the cell exhibits reactivity to a CTL, in other words, the protein of the invention or a tumor antigen peptide derived therefrom activates or induces a CTL. The activity and the method of determination thereof are as described in “1) Protein of the present invention” above.

The nucleic acid comprising the polynucleotide of the present invention may be either single- or double-stranded and may be either DNA or RNA. When the polynucleotide of the present invention is double stranded, an expression vector for expressing the protein of the present invention can be constructed by incorporating the above-mentioned polynucleotide into an expression vector. Thus, the nucleic acid of the present invention encompasses a recombinant expression vector constructed by inserting a double-stranded polynucleotide of the present invention to an expression vector.

A suitable expression vector can be selected depending on the host to be used, purposes and the like, and includes plasmids, phage vectors, virus vectors, and the like.

When the host is Escherichia coli, the vector may be a plasmid vector such as pUC118, pUC119, pBR322 and pCR3; or a phage vector such as λZAPII and λgt11. When the host is yeast, the vector may be pYES2, pYEUra3 and the like. When the host is an insect cell, the vector may be pAcSGHis NT-A and the like. When the host is an animal cell, the vector may be a plasmid vector such as pCEP4, pKCR, pCDM8, pGL2, pcDNA3.1, pRc/RSV and pRc/CMV; or a virus vector such as retrovirus vector, adenovirus vector and adeno-associated virus vector.

The expression vector may optionally contain a factor(s) such as promoter capable of inducing expression, a gene encoding a signal sequence, a marker gene for selection and terminator.

Furthermore, the expression vector may contain an additional sequence for expressing the protein as a fusion protein with thioredoxin, His-tag, GST (glutathione S-transferase), or the like, so as to facilitate isolation and purification of the protein. The vector usable in such a case includes a GST fusion protein vector containing an appropriate promoter (lac, tac, trc, trp, CMV, SV40 early promoter and the like) that functions in a host cell, such as pGEX4T; a vector containing Tag sequence (Myc, His and the like) such as pcDNA3.1/Myc-His; and a vector capable of expressing a fusion protein with thioredoxin and His such as pET32a.

By transforming a host cell with the expression vector obtained in the above, a transformant containing the vector of the present invention can be prepared.

The host cell usable herein includes Escherichia coli, yeast, insect cells and animal cells. Examples of Escherichia coli include strains of E. coli K-12 such as HB101, C600, JM109, DH5α and AD494 (DE3). Examples of yeast include Saccharomyces cerevisiae. Examples of animal cells include L929, BALB/c3T3, C127, CHO, COS, Vero, Hela and 293-EBNA cells. Examples of insect cells include sf9.

Introduction of an expression vector into a host cell can be done using a conventional method suited for the respective host cell above. Specifically, it can be done with calcium phosphate method, DEAE-dextran method, electroporation method, or a method using lipid for gene transfer (Lipofectamine, Lipofectin; Gibco-BRL). Following the introduction, the cell is cultured in a conventional medium containing a selection marker, whereby the transformant containing the expression vector can be selected.

The protein of the invention can be produced by culturing the transformant under appropriate conditions. The resultant protein may be further isolated and purified according to standard biochemical procedures. The purification procedure includes salting out, ion exchange chromatography, absorption chromatography, affinity chromatography, gel filtration chromatography, and the like. When the protein of the present invention is expressed as a fusion protein with thioredoxin, His tag, GST, or the like, as mentioned above, it can be isolated and purified by an appropriate purification procedure making use of the characteristics of such a fusion protein or tag.

### (2) Polynucleotide Encoding the Peptide of the Present Invention and Nucleic Acid Comprising the Same

As mentioned above, a nucleic acid comprising a polynucleotide encoding the peptide of the present invention falls within the scope of the nucleic acid of the present invention.

The polynucleotide encoding the peptide of the present invention may be either DNA or RNA and single- or double-stranded. The polynucleotide encoding the peptide of the present invention can be easily prepared on the basis of information about the amino acid sequence of the peptide or DNA encoding the same. Specifically, it can be prepared by a conventional method such as DNA synthesis or amplification by PCR.

Specifically, the polynucleotide encoding the peptide of the present invention includes a polynucleotide encoding the epitope peptide as mentioned above.

The nucleic acid comprising the polynucleotide encoding the peptide of the present invention may be either single- or double-stranded and also either DNA or RNA. When the polynucleotide of the present invention is double-stranded, a recombinant expression vector for expressing the peptide (epitope peptide) of the present invention can be constructed by introducing the above-mentioned polynucleotide into an expression vector.

The expression vector, host cell, method for transforming a host cell, and the like herein used are similar to those described in (1) above.

### 4) Antigen-Presenting Cell of the Present Invention

An antigen-presenting cell can be prepared by bringing a cell having an antigen-presenting ability into contact in vitro with any one of the protein, peptide and nucleic acid of the present invention as mentioned above. Specifically, the present invention provides a method of preparing an antigen-presenting cell characterized in that a cell having an antigen-presenting ability isolated from a tumor patient is brought into contact in vitro with any one of the protein, peptide and nucleic acid of the present invention and the antigen presenting cell prepared thereby.

In this context, the “cell having an antigen-presenting ability” is not limited to a particular cell and may be any cell that expresses on the surface an HLA antigen capable of presenting the peptide of the present invention; however, dendritic cells known to have especially high antigen-presenting ability are preferred.

Further, any of the protein, peptide and nucleic acid of the present invention may be used for preparing the antigen-presenting cell of the present invention from a cell having an antigen-presenting ability.

The antigen-presenting cell of the present invention can be prepared by isolating, from a tumor patient, cells having an antigen-presenting ability, pulsing the cells in vitro with the protein or peptide of the present invention, and allowing the cells to present a complex between an HLA antigen and the peptide of the present invention (Cancer Immunol. Immunother., 46: 82, 1998; J. Immunol. 158: p 1796, 1997; Cancer Res., 59:1184, 1999). When dendritic cells are used, the antigen-presenting cell of the present invention may be prepared, for example, by isolating lymphocytes from peripheral blood of a tumor patient using Ficoll method, removing non-adherent cells, incubating the adherent cells in the presence of GM-CSF and IL-4 to induce dendritic cells, and incubating and pulsing the dendritic cells with the protein or peptide of the present invention.

When the antigen-presenting cell of the present invention is prepared by introducing the nucleic acid of the present invention into the cell having an antigen-presenting ability, the nucleic acid may be in the form of DNA or RNA. In particular, DNA may be used according to the teaching in Cancer Res., 56:5672, 1996 or J. Immunol., 161: p 5607, 1998, and RNA according to the teaching in J. Exp. Med., 184:p 465, 1996, for example.

The antigen presenting cell of the present invention is characterized in that the cell presents a complex between the peptide of the present invention and an HLA antigen, and specifically includes an antigen presenting cell which is a dendritic cell and presents a complex between the peptide consisting of the amino acid sequence of SEQ ID NO: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 or 183 (preferably the amino acid sequence of 158, 22, 23, 26, 27, 198, 200, 201, 177, 178, 179 or 183) and HLA-A24 antigen on the cell surface. Such an antigen presenting cell can be prepared by isolating cells having an antigen-presenting ability from a HLA-A24⁺ prostate cancer patient, pulsing the cells in vitro with the peptide consisting of the amino acid sequence of SEQ ID NO: SEQ ID NO: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 or 183 (preferably the amino acid sequence of 158, 22, 23, 26, 27, 198, 200, 201, 177, 178, 179 or 183), and allowing the cells to present a complex between the peptide and HLA-A24 antigen.

### 5) CTL of the Present Invention

Any one of the protein, peptide and nucleic acid of the present invention can be used to induce a CTL in vitro when brought into contact with peripheral blood lymphocytes. Specifically, the present invention provides a method of inducing a CTL wherein peripheral blood lymphocytes from a tumor patient are brought into contact in vitro with any one of the protein, peptide and nucleic acid of the present invention and the CTL induced thereby.

For melanoma, adoptive immunotherapy has shown a therapeutic effect, wherein tumor-infiltrating T cells obtained from a patient were cultured ex vivo in large quantities and returned into the same patient (J. Natl. Cancer. Inst., 86: 1159, 1994). Further, in mouse melanoma, suppression of metastasis has been observed by stimulating splenocytes with a tumor antigen peptide TRP-2 in vitro to induce proliferation of a CTL specific to the tumor antigen peptide, and administering the CTL to a melanoma-grafted mouse (J. Exp. Med., 185:453, 1997). This resulted from the in vitro proliferation of a CTL that specifically recognizes the complex between an HLA antigen on antigen-presenting cells and the tumor antigen peptide. Accordingly, a therapeutic method comprising stimulating in vitro peripheral blood lymphocytes from a patient with the protein, peptide or nucleic acid of the present invention to proliferate a tumor-specific CTL, and returning the CTL into the patient is believed to be effective.

The CTL used in the adoptive immunotherapy can be prepared by isolating peripheral blood lymphocytes from a patient and stimulating the lymphocytes in vitro with the protein, peptide or nucleic acid of the present invention (Journal of Experimental Medicine 1999, 190:1669).

The CTL of the invention is characterized in that it is induced by bringing peripheral blood lymphocytes into contact in vitro with any one of the protein, peptide and nucleic acid of the present invention, and may be either a single CTL clone or CTL mixture (group) composed of various CTL clones. A specific example of such a CTL is that specifically recognizing a complex between the peptide consisting of the amino acid sequence of SEQ ID NO: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 or 183 and HLA-A24 antigen. More preferable example of such a CTL is that specifically recognizing a complex between the peptide consisting of the amino acid sequence of SEQ ID NO: 158, 22, 23, 26, 27, 198, 200, 201, 177, 178, 179 or 183 and HLA-A24 antigen.

### 6) Pharmaceutical Composition of the Present Invention

The protein, peptide, nucleic acid, antigen presenting cell and CTL of the present invention as mentioned above can be an active ingredient of a pharmaceutical composition in an appropriate form for each substance. The pharmaceutical composition of the present invention can be an active ingredient of an inducer of CTL, that is, a cancer vaccine, which is described in detail below.

### (1) Inducer of CTL Comprising the Protein of the Invention as an Active Ingredient

The protein of the present invention (Lengsin, BJ-TSA-9, C20orf42, BUB1, C10orf3, HIFPH3) has an activity of inducing CTLs and therefore, the protein of the present invention can be used as an active ingredient of a medicament for the treatment or prevention of tumor (cancer vaccine). Thus, the inducer of CTL comprising the protein of the present invention as an active ingredient exerts a therapeutic or preventive effect on tumor. The protein, when administered to a tumor patient, is incorporated by antigen-presenting cells and intracellularly degradated; the resultant tumor antigen peptide(s) generated by the intracellular degradation binds to an HLA antigen to form a complex; the complex is then presented on the surface of antigen-presenting cells; and a CTL specific for the complex efficiently proliferates in the body and destroys the tumor cells. In this way, treatment or prevention of tumor is achieved.

The inducer of CTL comprising the protein of the present invention as an active ingredient can be administered to any tumor patient who is positive for Lengsin, BJ-TSA-9, C20orf42, BUB1, C10orf3 or HIFPH3 protein.

Specifically, the inducer of CTL comprising Lengsin as an active ingredient can be used for the prevention or treatment of cancer (tumor) such as lung adenocarcinoma, lung squamous cell carcinoma or gastric cancer.

The inducer of CTL comprising BJ-TSA-9 as an active ingredient can be used for the prevention or treatment of cancer (tumor) such as leukemia, lung adenocarcinoma, lung squamous cell carcinoma, small cell lung cancer, oral cancer, gastric cancer, pancreas cancer or lymphoma.

The inducer of CTL comprising C20orf42 as an active ingredient can be used for the prevention or treatment of cancer (tumor) such as lung squamous cell carcinoma, lung adenocarcinoma, liver cancer, gastric cancer, leukemia, tissues of malignant lymphoma, rectal cancer, colon cancer or pancreas cancer.

The inducer of CTL comprising BUB1 as an active ingredient can be used for the prevention or treatment of cancer (tumor) such as breast cancer, lung adenocarcinoma, lung squamous cell carcinoma, ovarian cancer, oral squamous cell carcinoma, renal cancer, large bowel cancer (colon cancer, rectal cancer), gastric cancer, pancreas cancer, liver cancer, leukemia, lymphoma or melanoma.

The inducer of CTL comprising C10orf3 as an active ingredient can be used for the prevention or treatment of cancer (tumor) such as breast cancer, colon cancer, rectal cancer, renal cancer, gastric cancer, ovarian cancer, liver cancer, pancreas cancer, lung squamous cell carcinoma, lung adenocarcinoma, small cell lung cancer or melanoma.

The inducer of CTL comprising HIFPH3 as an active ingredient can be used for the prevention or treatment of cancer (tumor) such as breast cancer, colon cancer, gastric cancer, renal cancer, pancreas cancer, liver cancer, lung adenocarcinoma or lung squamous cell carcinoma.

The inducer of CTL comprising the protein of the present invention as an active ingredient may be administered as a mixture with, or together with, a pharmaceutically acceptable carrier, for example, an appropriate adjuvant, so that cellular immunity can be established effectively.

Examples of adjuvant applicable include those described in a literature (Clin. Microbiol. Rev., 7:277-289, 1994). Specifically, the followings are contemplated: a component derived from a microorganism or derivatives thereof, cytokines, a component derived from a plant or derivatives thereof, a component derived from a marine organism or derivatives thereof, mineral gels such as aluminium hydroxide, surfactants such as lysolecithin and Pluronic® polyols, polyanion, peptide, oil emulsion (emulsion preparation) and the like. In addition, liposomal preparations, particulate preparations in which the ingredient is bound to beads having a diameter of several μm, preparations in which the ingredient is attached to lipids, microsphere preparations, and microcapsules are also contemplated.

In this context, the “component derived from a microorganism or derivatives thereof” can be specifically classified into (1) killed bacteria, (2) Cell Wall Skeleton (hereinafter, “CWS”) derived from bacteria, and (3) a particular component derived from a microorganism and derivatives thereof.

(1) Examples of the killed bacteria include powdery hemolytic streptococcus (e.g., Picibanil®, Chugai Co., Ltd.), cocktail of killed bacterium suspension (e.g., Broncasma Berna®, Sanwa Kagaku Kenkyusho Co., Ltd) or killed bacteria of Mycobacterium tuberculosis.

(2) Examples of CWS derived from bacteria include CWS from Microbacterium (e.g., Mycobacterium bovis CWS), CWS from Nocardia (e.g., Nocardia rubra CWS), Corynebacterium CWS.

(3) Examples of a particular component derived from a microorganism and derivatives thereof include microorganism-derived polysaccharides such as polysaccharides from Mycobacterium tuberculosis (e.g., Ancer®, Zeria Pharmaceutical Co., Ltd.); polysaccharides from Basidiomycetes (Lentinan®, Ajinomoto, Co., Ltd.; Krestin®, Sankyo, Co., Ltd.; Basidiomycetes, Coriolus versicolor (Fr) Quel); muramyl dipeptide (MDP) associated compounds; lipopolysaccharides (LPS); lipid A (MPL) associated compounds; glycolipids trehalose dimycolate (TDM); bacterium DNA (e.g., CpG oligonucleotide); and derivatives thereof.

These microorganism-derived components and derivatives thereof can be available from commercial source or can be produced and isolated according to the methods described in known literatures (e.g., Cancer Res., 33, 2187-2195 (1973); J. Natl. Cancer Inst., 48, 831-835 (1972), J. Bacteriol., 94, 1736-1745 (1967); Gann, 69, 619-626 (1978), J. Bacteriol., 92, 869-879 (1966) or J. Natl. Cancer Inst., 52, 95-101 (1974)).

The term “cytokine”, for example, refers to IFN-α, IL-12, GM-CSF, IL-2, IFN-γ, IL-18 or IL-15. The cytokine may be a product of nature or genetic engineering. When the cytokine is commercially available, one can pursue and use the same. Alternatively, cytokine can be prepared recombinantly by cloning a desired gene in a conventional manner on the basis of the base sequence registered with database such as GenBank, EMBL or DDBJ, ligating the gene into an appropriate expression vector, transforming a host cell with the resultant recombinant expression vector, and allowing the cell to express and produce the intended cytokine.

Examples of the “component derived from a plant or derivatives thereof” include saponin-derived component Quil A (Accurate Chemical & Scientific Corp), QS-21 (Aquila Biopharmaceuticals Inc.), or glycyrrhizin (SIGMA-ALDRICH, etc.).

Examples of the “component derived from a marine organism or derivatives thereof” include sponge-derived glycolipid α-galactosylceramide.

Examples of oil emulsion (emulsion preparation) include emulsion preparations of water-in-oil type (w/o), oil-in-water type (o/w) and water-in-oil-in-water type (w/o/w).

In the water-in-oil type (w/o) emulsion preparation, an active ingredient is dispersed in water used as the disperse phase. In the oil-in-water type (o/w) emulsion preparation, an active ingredient is dispersed in water used as the disperse medium. Further, in the water-in-oil-in-water type (w/o/w) emulsion preparation, an active ingredient is dispersed in water which is the most internal phase. Such emulsion preparations can be produced in accordance with the teaching in, for example, JP-A-8-985, JP-A-9-122476, or the like.

The “liposomal preparation” refers to a microparticle wherein an active ingredient is encapsulated in a liposome having a lipid bilayer structure in the water phase or within the lipid bilayer. Representative lipids for preparation of the liposome include phosphatidyl choline and sphingomyelin. Dicetyl phosphate, phosphatidic acid, phosphatidyl serine or the like that confers charge may also be added for stabilization of liposomes. The method of producing liposomes include ultrasonic method, ethanol injection method, ether injection method, reverse phase evaporation method, French press extraction method, and the like.

The “microsphere preparation” refers to a microparticle composed of a homogeneous polymer matrix wherein an active ingredient is dispersed in the matrix. The matrix can be composed of a biodegradable polymer such as albumin, gelatin, chitin, chitosan, starch, polylactic acid, polyalkyl cyanoacrylate, and the like. The microsphere preparation can be prepared by any of known methods without limitation, including those described in literatures (Eur. J. Pharm. Biopharm. 50:129-146, 2000; Dev. Biol. Stand. 92:63-78, 1998; Pharm. Biotechnol. 10:1-43, 1997, etc.).

The “microcapsule preparation” refers to a microparticle containing an active ingredient as a core substance which is enveloped with a film. The coating material used for the film includes a film-forming polymer such as carboxymethylcellulose, cellulose acetate phthalate, ethyl cellulose, gelatin, gelatin/acacia, nitrocellulose, polyvinyl alcohol, hydroxypropyl cellulose, and the like. The microcapsule preparation can be prepared by coacervation method, surface polymerization, and the like.

Administration may be achieved, for example, intradermally, subcutaneously, intramuscularly, or intravenously. Although the dosage of the protein of the present invention in the formulation to be administered may be adjusted as appropriate depending on, for example, the disease to be treated, the age and the body weight of the patient, it is usually within the range of 0.0001-1000 mg, preferably, 0.001-100 mg, more preferably 0.01-10 mg, which can be administered once in every several days to every several months.

### (2) Inducer of CTL Comprising a Peptide of the Present Invention as an Active Ingredient

The peptide of the present invention has an activity of inducing a CTL. The induced CTL can exert an anti-tumor effect through cytotoxic action or production of lymphokines. Accordingly, the peptide of the present invention can be used as an active ingredient of a medicament for the treatment or prevention of tumor (cancer vaccine). When an inducer of CTL comprising the peptide of the present invention as an active ingredient is administered to a tumor patient, the peptide of the present invention is presented to an HLA antigen in antigen-presenting cells. Then, a CTL specific for the presented binding complex between the HLA antigen and the peptide of the present invention proliferates, which in turn destroys tumor cells. In this way, the treatment or prevention of tumor in a patient can be achieved.

The inducer of CTL comprising the peptide of the present invention as an active ingredient can be administered to any tumor patient who is positive for the protein of the invention. Specifically, it can be used for the prevention or treatment of cancer (tumor) as described in 6)-(1) above.

The inducer of CTL comprising the peptide of the present invention as an active ingredient may comprise as an active ingredient a single CTL epitope (peptide of the present invention) or a epitope peptide composed of the peptide of the present invention and other peptide(s) (CTL epitope or helper epitope) ligated together. Recently, an epitope peptide composed of multiple (plural) CTL epitopes (antigen peptides) ligated together has been shown to have an activity of inducing CTLs efficiently. For example, it has been reported that an epitope peptide of approximately 30-mer composed of CTL epitopes each restricted to HLA-A2-, -A3-, -A11 or B53 originated from tumor antigen protein PSA ligated induced CTLs specific for respective CTL epitopes (Journal of Immunology 1998, 161: 3186-3194). In addition, it has been reported that an epitope peptide composed of a CTL epitope and a helper epitope ligated can induce a CTL efficiently. When the peptide of the present invention is administered in the form of an epitope peptide, the peptide is incorporated into antigen-presenting cells; then the antigen peptide generated by intracellular degradation binds to an HLA antigen to form a complex; the complex is presented on the surface of antigen-presenting cells in high density; a CTL specific for the complex efficiently proliferates in the body, and destroys tumor cells. In this way, treatment or prevention of tumor is achieved.

Specific examples of the inducer of CTL comprising the peptide of the present invention as an active ingredient include one comprising as an active ingredient the tumor antigen peptide consisting of the amino acid sequence of any one of SEQ ID NOS: 13 to 201. Preferable example of the inducer of CTL is one comprising as an active ingredient the peptide consisting of the amino acid sequence of any one of SEQ ID NOS: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 and 183. More preferable example of the inducer of CTL is one comprising as an active ingredient the peptide consisting of the amino acid sequence of any one of SEQ ID NOS: 158, 22, 23, 26, 27, 198, 200, 201, 177, 178, 179 and 183.

The inducer of CTL comprising the peptide of the present invention as an active ingredient may be administered in a mixture with, or together with, a pharmaceutically acceptable carrier, for example, an appropriate adjuvant, so that cellular immunity can be established effectively.

Examples of adjuvant applicable include those described in a literature (Clin. Microbiol. Rev., 7:277-289, 1994). Specifically, the followings are contemplated: a component derived from a microorganism or derivatives thereof, cytokines, a component derived from a plant or derivatives thereof, a component derived from a marine organism or derivatives thereof, mineral gels such as aluminium hydroxide, surfactants such as lysolecithin and Pluronic® polyols, polyanion, peptides, oil emulsion (emulsion preparation) and the like. In addition, liposomal preparations, particulate preparations in which the ingredient is bound to beads having a diameter of several μm, preparations in which the ingredient is attached to lipids, microsphere preparations, and microcapsules are also contemplated. Concrete examples of these adjuvants are the same as those descried in the above “6)-1) Inducer of CTL comprising the protein of the invention as an active ingredient”.

Administration may be achieved, for example, intradermally, subcutaneously, intramuscularly, or intravenously. Although the dosage of the peptide of the present invention in the formulation to be administered may be adjusted as appropriate depending on, for example, the disease to be treated, the age and the body weight of the patient, it is usually within the range of 0.0001-1000 mg, preferably 0.001-1000 mg, more preferably 0.1-10 mg, which can be administered once in every several days to every several months.

### (3) Inducer of CTL Comprising the Nucleic Acid of the Present Invention as an Active Ingredient

The nucleic acid of the present invention has an activity of inducing a CTL and thus can be an active ingredient of a medicament for the treatment or prevention of tumor (cancer vaccine). The inducer of CTL comprising the nucleic acid of the present invention as an active ingredient, when administered, can exert a therapeutic or preventive effect on tumor through the expression of the nucleic acid.

For example, when the nucleic acid of the present invention incorporated into an expression vector is administered to a tumor patient in the following manner, the tumor antigen protein is highly expressed in antigen-presenting cells. Thereafter, tumor antigen peptides generated by intracellular degradation form a complex with an HLA antigen; the complex is then presented on the surface of antigen-presenting cells in high density; and tumor-specific CTLs proliferate in the body efficiently and destroy tumor cells. In this way, treatment or prevention of tumor is achieved.

The inducer of CTL comprising the nucleic acid of the present invention as an active ingredient can be administered to any tumor patient who is positive for the protein of the invention. Specifically, it can be used for the prevention or treatment of cancer (tumor) as described in 6)-(1) above.

Administration and introduction of the nucleic acid of the present invention into cells may be achieved by using a viral vector or by other procedures (Nikkei-Science, April, 1994, pp. 20-45; Gekkan-Yakuji, 36(1), 23-48 (1994); Jikken-Igaku-Zokan, 12(15), 1994, and references cited therein).

The method of introducing a viral vector may comprises incorporation of the DNA of the present invention into a DNA or RNA virus such as retrovirus, adenovirus, adeno-associated virus, herpesvirus, vaccinia virus, poxvirus, poliovirus, or Sindbis virus, and introduction of the virus into cells. Above all, the method involving a retrovirus, adenovirus, adeno-associated virus, or vaccinia virus is particularly preferred.

Other than the above method, a method wherein an expression plasmid is directly injected intramuscularly (DNA vaccination), liposome method, Lipofectin method, microinjection, calcium phosphate method and electroporation are exemplified, and among them DNA vaccination and liposome method are particularly preferred.

The nucleic acid of the present invention can act as a medicament in practice in, for example, an in vivo method wherein the nucleic acid is directly introduced into the body, or an ex vivo method wherein the nucleic acid is introduced extracorporeally into a certain cell obtained from a human subject and the cell is reintroduced into the body of the subject (Nikkei-Science, April, 1994, pp. 20-45; Gekkan-Yakuji, 36(1), 23-48 (1994); Jikkenn-Igaku-Zokan, 12(15), 1994; and references cited therein). An in vivo method is more preferred.

In case of the in vivo method, administration can be effected through any appropriate route depending on the disease and symptom to be treated and other factors. For example, it may be administered via intravenous, intraarterial, subcutaneous, intracutaneous, intramuscular route, or the like. When administered in the in vivo method, the nucleic acid of the present invention may be formulated into a liquid preparation, and typically into an injectable form containing the nucleic acid of the present invention as an active ingredient, to which a pharmaceutically acceptable carrier may also be added, if necessary. As to a liposome or membrane-fused liposome (such as Sendai virus (HVJ)-liposomes) containing the nucleic acid of the present invention, a liposomal formulation in the form of suspension, frozen preparation, centrifugally-concentrated frozen preparation, or the like may be accepted.

Although the dosage of the nucleic acid of the present invention in the formulation to be administered may be adjusted as appropriate depending on, for example, the disease to be treated, the age and the body weight of the patient, it is usually, as the amount of polynucleotide in the nucleic acid, within the range of 0.0001-100 mg, preferably, 0.001-10 mg, which can be administered once in every several days to every several months.

Recently, a polynucleotide encoding an epitope peptide composed of multiple (plural) CTL epitopes (tumor antigen peptides) ligated or of a CTL epitope(s) and a helper epitope(s) ligated has been shown to induce CTLs in vivo efficiently. For example, it is reported that a DNA (minigene) encoding an epitope peptide composed of six kinds of HBV-originated HLA-A2-restricted antigen peptides, three kinds of HLA-A11-restricted antigen peptides and a helper epitope ligated induced in vivo CTLs directed to the respective epitopes efficiently (Journal of Immunology 1999, 162: 3915-3925).

Accordingly, a polynucleotide prepared by ligating one or more polynucleotides each encoding the peptide of the present invention, and optionally other polynucleotide(s) encoding different peptide(s), can be an active ingredient of an inducer of CTL when introduced into an appropriate expression vector. Such an inducer of CTL may be applied in the same administration manner or form that described above.

### (4) Inducer of CTL Comprising the Antigen Presenting Cell of the Present Invention as an Active Ingredient

The antigen presenting cell of the present invention has an activity of inducing a CTL and thus can be an active ingredient of a medicament for the treatment or prevention of tumor (cancer vaccine). The inducer of CTL comprising the antigen presenting cell of the present invention as an active ingredient can exert a therapeutic or preventive effect on tumor through administration of the antigen presenting cell to a tumor patient.

The inducer of CTL comprising the antigen presenting cell of the present invention as an active ingredient can be administered to any tumor patient who is positive for the protein of the invention. Specifically, it can be used for the prevention or treatment of cancer (tumor) as described in 6)-(1) above.

The inducer of CTL comprising the antigen presenting cell as an active ingredient preferably contains physiological saline, phosphate buffered saline (PBS), medium, or the like to stably maintain the antigen-presenting cell. It may be administered, for example, intravenously, subcutaneously, or intradermally. Dosage of the inducer is exemplified in the previous literature. Reintroduction of the inducer of CTL comprising the antigen-presenting cell as an active ingredient into a patient positive for the protein of the invention can cause efficient induction of a specific CTL in the body of the patient, and, result in the treatment of tumor.

### (5) Cancer Vaccine Comprising the CTL of the Present Invention as an Active Ingredient

The CTL of the present invention has a cytotoxic activity against tumor cells and thus can be an active ingredient of a medicament for the treatment or prevention of tumor (cancer vaccine).

The pharmaceutical composition comprising the CTL of the present invention as an active ingredient for treating or preventing tumor can be administered to any tumor patient who is positive for the protein of the invention. Specifically, it can be used for the prevention or treatment of cancer (tumor) as described in 6)-(1) above.

The pharmaceutical composition comprising the CTL of the present invention as an active ingredient for treating or preventing tumor preferably contains physiological saline, phosphate buffered saline (PBS), medium, or the like to stably maintain the CTL. It may be administered, for example, intravenously, subcutaneously, or intradermally. Reintroduction of the pharmaceutical composition comprising the CTL of the present invention as an active ingredient into a patient positive for the protein of the invention can cause promotion of the cytotoxic activity of CTLs against tumor cells in the body of the patient, followed by destruction of the tumor cells, and result in treatment of tumor.

### 7) Antibody Against the Peptide of the Present Invention

The present invention provides an antibody specifically binding to the peptide of the present invention. The antibody of the present invention is not limited in terms of the form, and may be a polyclonal or monoclonal antibody raised against the peptide of the present invention.

The antibody of the present invention may not be limited in any sense provided that it specifically binds to the peptide of the present invention as mentioned above, and the specific example is an antibody that specifically binds to a tumor antigen peptide consisting of the amino acid sequence of any one of SEQ ID NOS: 13 to 201, preferably the amino acid sequence of SEQ ID NO: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 or 183, more preferably, 158, 22, 23, 26, 27, 198, 200, 201, 177, 178, 179 or 183.

Methods of preparing an antibody are well known in the art and the antibody of the present invention can be prepared according to any one of these conventional methods (Current protocols in Molecular Biology edit. Ausubel et al. (1987) Publish. John Wiley and Sons. Section 11.12-11.13, Antibodies; A Laboratory Manual, Lane, H, D. et al., ed., Cold Spring Harbor Laboratory Press, New York 1989).

Specifically, the antibody of the present invention can be obtained by immunizing a non-human animal such as rabbit using the peptide of the present invention (e.g., a peptide consisting of the amino acid sequence of any one of SEQ ID NOS: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 and 183) as an immunogen, and recovering the antibody from serum of the immunized animal in a conventional manner. When the antibody is monoclonal, it can be obtained by immunizing a non-human animal such as mouse with the peptide of the present invention (e.g., a peptide consisting of the amino acid sequence of any one of SEQ ID NOS: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 and 183), subjecting the resultant splenocyte to cell fusion with a myeloma cell to prepare a hybridoma cell, and recovering the antibody from the hybridoma cell (Current protocols in Molecular Biology edit. Ausubel et al. (1987) Publish. John Wiley and Sons. Section 11.4-11.11).

The antibody against the peptide of the present invention can also be produced while enhancing the immunological response using different adjuvants depending on the host. Examples of the adjuvants include Freund adjuvant; mineral gels such as aluminium hydroxide; surfactants such as lysolecithin and Pluronic® polyol, polyanion, peptides, oil emulsion, keyhole limpet hemocyanin and dinitrophenol; human adjuvants such as BCG (Bacille de Calmette-Guerin) and Corynebacterium.

As mentioned above, an antibody that recognizes the peptide of the present invention and an antibody that neutralizes the activity of the peptide may easily be prepared by immunizing an animal in a conventional manner. The antibody may be used in affinity chromatography, immunological diagnostic method, and the like. Immunological diagnostic method may be selected as appropriate from immunoblotting, radioimmunoassay (RIA), enzyme-linked immunosorbent assay (ELISA), fluorescent or luminescent assay, and the like. The immunological diagnostic method would be effective for diagnosing cancer which expresses the gene encoding the protein of the present invention.

### 8) HLA Monomer, HLA Dimer, HLA Tetramer and HLA Pentamer of the Present Invention

The present invention also provides an HLA monomer, HLA dimer, HLA tetramer or HLA pentamer comprising the tumor antigen peptide of the present invention and an HLA antigen.

In cancer immunotherapy, a significant indicator for selecting a patient highly responsive to a tumor antigen (tumor antigen peptide), monitoring the therapeutic effect, or evaluating the suitability to treatment can be obtained through examination of frequency or amount of CTL precursor cells directed to the tumor antigen (tumor antigen peptide) in a patient before initiation of treatment, or examination of frequency or amount of CTLs in a patient undergoing treatment with the tumor antigen (tumor antigen peptide). An HLA monomer, HLA dimer, HLA tetramer and HLA pentamer each comprising a tumor antigen peptide and an HLA antigen are useful as a reagent in detection of a CTL specific for the antigen (antigen peptide), specifically, in the measurement of frequency or amount of the CTL.

As used herein, the HLA tetramer refers to a tetramer prepared by biotinylating a complex composed of HLA antigen α-chain and β2-microglobulin associated with a peptide (antigen peptide) (HLA monomer), and allowing to bind to avidin for tetramerization (Science 279: 2103-2106 (1998); and Science 274: 94-96 (1996)).

The HLA monomer is a monomer that is used in the preparation of the above-mentioned HLA tetramer and is formed by biotinylating an associate of HLA antigen α-chain, β2-microglobulin and an antigen peptide.

The HLA dimer is a dimer prepared by fusing HLA antigen α-chain and Ig (immunoglobulin, for example, IgG1), and binding the resultant fusion to β2-microglobulin and an antigen peptide (Proc. Natl. Acad. Sci. USA 90: 6671-6675 (1993)). The CTL specific for the antigen peptide bound to the HLA dimer can be detected by, for example, allowing labeled anti-IgG1 antibody to bind to the IgG1.

The HLA pentamer is a recently developed technique and refers to a pentamer comprising five molecules of a complex of an HLA antigen and an antigen peptide polymerized through Coiled-Coil domain. Since the complex of an HLA antigen and an antigen peptide can be labeled with fluorescence or the like, the analysis can be carried out by flow cytometry or the like similarly to the HLA tetramer (see, http://www.proimmune.co.uk/).

The HLA-monomer, dimer, tetramer and pentamer as mentioned above are all available by custom production from a manufacture such as ProImmune or BD Biosciences. At present, HLA tetramers and the like which comprise different antigen peptides are commercially available (Medical & Biological Laboratories Co., Ltd., etc.).

Examples of the HLA monomer, dimer, tetramer and pentamer of the present invention, specifically, include a HLA monomer, dimer, tetramer and pentamer each comprising the peptide consisting of the amino acid sequence of SEQ ID NO: 42, 43, 44, 45, 46, 47, 49, 59, 60, 61, 62, 63, 64, 65, 66, 67, 68, 69, 70, 71, 72, 73, 74, 75, 76, 77, 78, 158, 159, 160, 161, 15, 16, 21, 22, 23, 24, 25, 26, 27, 30, 195, 197, 198, 199, 200, 201, 177, 178, 179 and 183, preferably 158, 22, 23, 26, 27, 198, 200, 201, 177, 178, 179 and 183 and HLA-A24 antigen. Above all, an HLA tetramer or an HLA pentamer is preferred for detection of a CTL.

The HLA monomer, HLA tetramer and HLA pentamer are preferably labeled with fluorescence so that the bound CTL can be easily sorted out or detected by a known detection method such as flow cytometry, fluorescent microscopy, and the like. Examples include HLA monomers, tetramers and pentamers labeled with phycoerythrin(PE), fluorescein isothiocyanate (FITC), peridinyl chlorophyll protein (PerCP), allophycocyanin (APC), or the like.

Among HLA antigens which may be a component of the HLA monomer, dimer, tetramer and pentamer of the present invention, HLA-A24 antigen (α-chain of HLA-A24 antigen) can be cloned easily by a conventional method such as PCR on the basis of a known base sequence of HLA-A2402 disclosed in Genbank Accession No. M64740. HLA-A2 antigen (α-chain of HLA-A2 antigen) can be cloned easily by a conventional method such as PCR on the basis of a known base sequence of HLA-A0201 gene disclosed in Genbank Accession No. M84379.

The β2-microglobulin which is a component of the HLA monomer, dimer, tetramer and pentamer of the present invention is preferably originated from human. The human β2-microglobulin can be cloned easily by a conventional method such as PCR on the basis of a known base sequence of human β2-microglobulin disclosed in Genbank Accession No. AB021288.

The processes for preparing the HLA monomer, dimer, tetramer and pentamer are well known from the respective literatures mentioned above. The preparation will be hereinafter described briefly regarding the HLA tetramer of HLA-A24.

First, an appropriate host cell such as E. coli or a mammalian cell capable of expressing a protein is transformed with an HLA-A24 α-chain expression vector and a β2-microglobulin expression vector, and allowed to express them. E. coli (e.g., BL21) is preferably used here. The resultant HLA-A24 complex in a monomeric form and the peptide of the invention are then mixed to form a soluble HLA-peptide complex. The C-terminal sequence of HLA-A24 α-chain of the HLA-peptide complex is biotinylated with BirA enzyme. When the biotinylated HLA-peptide complex and fluorescently labeled avidin are mixed at the molar ratio of 4:1, an HLA tetramer is formed. It is preferred to purify the resulting protein in each step above by gel filtration or the like.

The HLA monomer, dimer, tetramer and pentamer described above may be used effectively as a detecting reagent for a CTL specific for a tumor antigen peptide derived from the protein of the invention.

The CTL-detecting reagent of the present invention can be used for the following purposes, for example.

1) To examine the frequency or amount of a CTL precursor for the tumor antigen peptide of the present invention before the initiation of treatment with the protein, peptide or nucleic acid of the present invention. In doing so, responsiveness of a patient to the tumor antigen peptide can be assessed.
2) To examine the frequency or amount of CTLs in a patient under treatment with the protein, peptide or nucleic acid of the present invention. In doing so, it becomes possible to conduct monitoring of a therapeutic effect, evaluation of suitability of treatment, confirmation of favorable progress of treatment, and the like.

Detection of a CTL can be carried out by, specifically, isolating a biological sample containing CTLs (e.g., PBMCs) from a subject patient, bringing the HLA tetramer or the like of the present invention into contact with the biological sample, and measuring the frequency or amount of the existing CTL specific for the peptide of the invention bound to the HLA tetramer by, for example, flow cytometry.

### 9) Disease Marker of the Invention

The present invention also provides a disease marker for cancer (tumor) making use of Lengsin, BJ-TSA-9, C20orf42, BUB1, C10orf3 or HIFPH3 gene, or a expression product thereof.

As described above, the inventors found that the expression levels and/or frequencies of Lengsin, BJ-TSA-9, C20orf42, BUB1, C10orf3 and HIFPH3 genes were significantly increased in cancer tissues compared to normal tissues. Antibodies recognizing those genes or expression products thereof (i.e., proteins) are, therefore, useful as disease markers for cancers expressing those genes.

Use of the antibody recognizing a polynucleotide of Lengsin, BJ-TSA-9, C20orf42, BUB1, C10orf3 or HIFPH3 gene or an expression product thereof (i.e., a protein) as a disease marker is explained hereinafter.

### (1) Disease Marker for Cancer and Application Thereof

### (1-1) Polynucleotide

As described above, human Lengsin, BJ-TSA-9, C20orf42, BUB1, C10orf3 and HIFPH3 genes are known in the art.

The present invention is, as already mentioned, based on the finding that the expression levels and/or frequencies of Lengsin, BJ-TSA-9, C20orf42, BUB1, C10orf3 and HIFPH3 genes were specifically increased in tissues of cancer patients compared to normal tissues, and also the idea that detection of presence or absence of expression, or expression levels, of those genes can provide specific determination of presence or absence of, severity of, or recovery from cancer and thus precise diagnosis of the cancer.

Accordingly, the present invention provides a polynucleotide useful as a tool (i.e., a disease marker) which allows diagnosis of presence or absence or severity of cancer in a test subject through detection of presence or absence of expression, or expression levels, of those genes in the test subject.

The “polynucleotide” herein used includes RNA and DNA and may be either single- or double-stranded. The “DNA” includes cDNA, genomic DNA and a synthetic DNA. The “RNA” includes total RNA, mRNA and a synthetic RNA. The disease marker of the present invention is characterized in that it consists of a polynucleotide and/or a complementary polynucleotide thereof, wherein the polynucleotide comprises at least 15 contiguous nucleotides from the base sequence of Lengsin, BJ-TSA-9, C20orf42, BUB1, C10orf3 or HIFPH3 gene.

Specifically, the disease marker of the present invention may consist of a polynucleotide and/or a complementary polynucleotide thereof, wherein the polynucleotide comprises at least 15 contiguous nucleotides from the base sequence of Lengsin gene (SEQ ID NO: 1), BJ-TSA-9 gene (SEQ ID NO: 3), C20orf42 gene (SEQ ID NO: 5), BUB1 gene (SEQ ID NO: 7), C10orf3 gene (SEQ ID NO: 9) or HIFPH3 gene (SEQ ID NO: 11).

In this context, the term “complementary polynucleotide (complementary strand, reverse strand)” refers to a polynucleotide which is complementary based on the base-pairing rules (e.g., A:T, G:C) to a full sequence of the polynucleotide consisting of any one of the base sequences of the above SEQ ID NOS or a partial sequence thereof comprising at least 15 contiguous nucleotides from any one of those base sequences, which is herein also referred to as “forward strand” for convenience. The “complementary strand” may be a sequence being completely complementary to a base sequence of a target forward strand, or a sequence being complementary enough to hybridize with the base sequence of the target forward strand under stringent conditions. The stringent conditions herein used can be determined on the basis of the melting temperature (Tm) of nucleic acids forming a complex or binding to a probe as described in literatures (Berger and Kimmel, 1987, “Guide to Molecular Cloning Techniques Methods in Enzymology”, Vol. 152, Academic Press, San Diego Calif.). For example, washing after hybridization can be conducted under a condition around “1×SSC, 0.1% SDS, 37° C. in usual. The complementary strand preferably remains bound to the target forward strand when washed under such washing conditions. More stringent hybridization conditions may involve washing under the conditions of around “0.5×SSC, 0.1% SDS, 42° C.” and still more stringent hybridization conditions involve washing conditions of around “0.1×SSC, 0.1% SDS, 65° C.”, although it is not limited thereto. Specifically, such a complementary strand may consist of a base sequence having a complete complementarity, or a homology of at least 90%, preferably 95% compared to a base sequence of a target forward strand.

The polynucleotide of a forward strand may have a full or partial sequence of the base sequence of Lengsin, BJ-TSA-9, C20orf42, BUB1, C10orf3 or HIFPH gene, or may be complementary to the base sequence of the above-mentioned complementary strand.

The polynucleotide of forward or complementary (reverse) strand as mentioned above can be used as a disease marker in either single-stranded or double-stranded form.

Specifically, the disease marker of the present invention may be a polynucleotide consisting of a base sequence (full sequence) of Lengsin, BJ-TSA-9, C20orf42, BUB1, C10orf3 or HIFPH3 gene, or a complementary sequence thereof. Alternatively, the disease marker may be a polynucleotide consisting of a partial sequence of the full sequence as mentioned above or a complementary sequence thereof as long as the polynucleotide selectively (specifically) recognizes Lengsin, BJ-TSA-9, C20orf42, BUB1, C10orf3 or HIFPH3 gene or a polynucleotide originated therefrom. The partial sequence may be a polynucleotide comprising at least 15 contiguous nucleotides selected as appropriate from the base sequence of the aforementioned full sequence or a complementary sequence thereof.

The term “selectively (specifically) recognizes” used herein means that, in the case of Northern blotting, Lengsin, BJ-TSA-9, C20orf42, BUB1, C10orf3 or HIFPH3 gene or a polynucleotide derived therefrom can be specifically detected and in the case of RT-PCR method, Lengsin, BJ-TSA-9, C20orf42, BUB1, C10orf3 or HIFPH3 gene or a polynucleotide derived therefrom is produced. However, the above definition is not restrictive and any criteria can be used as long as one ordinary skilled in the art can determine that the substance detected or a product generated by using the polynucleotide originates from any of those genes.

For example, the disease marker of the invention can be designed from the base sequence of Lengsin gene (SEQ ID NO: 1), BJ-TSA-9 gene (SEQ ID NO: 3), C20orf42 gene (SEQ ID NO: 5), BUB1 gene (SEQ ID NO: 7), C10orf3 gene (SEQ ID NO: 9) or HIFPH3 gene (SEQ ID NO: 11) by means of primer 3 (http://www.genome.wi.mit.edu/cgi-bin/primer/primer3.cgi) or a vector NTI (Infomax). Specifically, a candidate sequence obtained from the base sequence of Lengsin, BJ-TSA-9, C20orf42, BUB1, C10orf3 or HIFPH3 gene by using primer 3 or vector NTI software, or a sequence comprising at least a part of the candidate sequence, can be used as a primer or probe.

The disease marker of the present invention comprises at least 15 contiguous nucleotides in length as mentioned above, and depending on the application of the marker, the length can be appropriately selected and designated.

### (1-2) Polynucleotide as a Probe or Primer

Detection (diagnosis) of cancer of the present invention is conducted by examining presence or absence of expression, or expression level, of at least one of Lengsin, BJ-TSA-9, C20orf42, BUB1, C10orf3 and HIFPH3 genes in a biological sample from a test subject. Here, the disease marker of the present invention may be used as a primer which specifically recognizes and amplifies RNA expressed from any of those genes or a polynucleotide derived therefrom, or as a probe for detecting specifically such RNA or a polypeptide derived therefrom.

When the disease maker of the present invention is used as a primer for detecting cancer (i.e., used in genetic diagnosis), the primer may comprise generally 15 to 100 bp, preferably 15 or 50 bp, more preferably 15 to 35 bp in length. When used as a probe for detecting cancer, the probe may comprise generally 15 bp to 1 kp, preferably 100 bp to 1 kb in length.

The disease marker of the invention can be used as a primer or probe in a conventional manner in any of known methods for detecting specifically a particular gene such as Northern blotting, RT-PCR, in situ hybridization, and the like. Then, presence or absence of expression, or expression level, of Lengsin, BJ-TSA-9, C20orf42, BUB1, C10orf3 or HIFPH3 gene which is involved in cancer can be examined.

A sample to be examined may be total RNA prepared with a conventional method from a biological sample, such as a biopsy sample, of a target tissue of a test subject or polynucleotides prepared from the total RNA.

The expression level of Lengsin, BJ-TSA-9, C20orf42, BUB1, C10orf3 or HIFPH3 gene in a biological sample can be detected or quantified by using a DNA chip. Here, the disease marker of the invention may be used as a probe for the DNA chip (in the case of Gene Chip Human Genome U95 A, B, C, D, E (Affymetrix), used as a polynucleotide probe being 25 bp in length). When a DNA chip containing the disease marker of the invention as its probe is hybridized with labeled-DNA or RNA prepared from RNA of a biological sample, the disease marker of the invention (i.e., the probe) and the labeled DNA or RNA form a complex. By detecting the complex by means of the label of the labeled DNA or RNA, presence or absence of expression, or expression level, of Lengsin, BJ-TSA-9, C20orf42, BUB1, C10orf3 or HIFPH3 gene can be examined.

The DNA chip may contain one or more of the disease markers of the invention which can bind to any of Lengsin, BJ-TSA-9, C20orf42, BUB1, C10orf3 and HIFPH3 genes. When the DNA chip contains more than one markers, presence or absence expression, or expression level, can be examined simultaneously for more than one gene in one biological sample.

The disease marker of the present invention is useful for diagnosis or detection of cancer, that is, diagnosis of presence or absence or severity of cancer. Specifically, diagnosis of cancer using the disease marker can be conducted by evaluating the difference in the expression level of Lengsin, BJ-TSA-9, C20orf42, BUB1, C10orf3 or HIFPH3 gene in a biological sample of a tissue between a test subject and a healthy subject.

Here, the difference between the expression levels of both subjects may be presence or absence of expression, or alternatively, when the expression is observed in both subjects, may be at least 1.5-fold, preferably 2-fold, more preferably 3-fold.

A polynucleotide derived from Lengsin gene is specifically useful as a disease marker for lung adenocarcinoma, lung squamous cell carcinoma or gastric cancer.

A polynucleotide derived from BJ-TSA-9 gene is specifically useful as a disease marker for leukemia, lung adenocarcinoma, lung squamous cell carcinoma, small cell lung cancer, oral cancer, gastric cancer, pancreas cancer or lymphoma.

A polynucleotide derived from C20orf42 gene is specifically useful as a disease marker for lung squamous cell carcinoma, lung adenocarcinoma, liver cancer, gastric cancer, leukemia, malignant lymphoma tissues, rectal cancer, colon cancer or pancreas cancer.

A polynucleotide derived from BUB1 gene is specifically useful as a disease marker for breast cancer, lung adenocarcinoma, lung squamous cell carcinoma, ovarian cancer, oral squamous cell carcinoma, renal cancer, large bowel cancer (colon cancer, rectal cancer), gastric cancer, pancreas cancer, liver cancer, leukemia, lymphoma or melanoma.

A polynucleotide derived from C10orf3 gene is specifically useful as a disease marker for breast cancer, colon cancer, rectal cancer, renal cancer, gastric cancer, ovarian cancer, liver cancer, pancreas cancer, lung squamous cell carcinoma, lung adenocarcinoma, small cell lung cancer or melanoma.

A polynucleotide derived from HIFPH3 gene is specifically useful as a disease marker for breast cancer, colon cancer, gastric cancer, renal cancer, pancreas cancer, liver cancer, lung adenocarcinoma or lung squamous cell carcinoma.

### (1-3) Antibody

The present invention provides an antibody specifically recognizing an expression product of Lengsin, BJ-TSA-9, C20orf42, BUB1, C10orf3 or HIFPH3 gene (i.e., protein Lengsin, BJ-TSA-9, C20orf42, BUB1, C10orf3 or HIFPH3) which can be used as a disease marker for cancer.

Specifically, the present invention provides an antibody which specifically recognizes Lengsin, BJ-TSA-9, C20orf42, BUB1, C10orf3 or HIFPH3 protein having the amino acid sequence of SEQ ID NO: 2, 4, 6, 8, 10, or 12, respectively.

The present invention is, as already mentioned, based on the finding that the expression levels and/or frequencies of Lengsin, BJ-TSA-9, C20orf42, BUB1, C10orf3 and HIFPH3 genes were specifically increased in tissues of cancer patients compared to normal cells or tissues and also the idea that detection of presence or absence of expression, or expression levels, of those genes can provide specific determination of presence or absence of, severity of, or recovery from cancer and thus precise diagnosis of the cancer.

Accordingly, the antibody is useful as a tool (i.e., a disease marker) which allows diagnosis of presence or absence or severity of cancer for a test subject through detection of presence or absence of expression, or expression level, of such a protein in the test subject.

As described above, human Lengsin, BJ-TSA-9, C20orf42, BUB1, C10orf3 and HIFPH3 proteins are known in the art.

The antibody of the present invention is not limited in terms of the form, and may be a polyclonal or monoclonal antibody raised against Lengsin, BJ-TSA-9, C20orf42, BUB1, C10orf3 or HIFPH3. Further, the antibody of the present invention includes that having an affinity to a polypeptide consisting of as least, generally 8, preferably 15, more preferably 20 contiguous amino acids from the amino acid sequence of any of those proteins.

Methods of preparing such an antibody are well known in the art and the antibody of the invention can be prepared according to any one of those conventional methods (Current protocols in Molecular Biology edit. Ausubel et al. (1987) Publish. John Wiley and Sons. Section 11.12-11.13). Specifically, when the antibody is polyclonal, it can be obtained by immunizing a non-human animal such as rabbit using as an immunogen Lengsin, BJ-TSA-9, C20orf42, BUB1, C10orf3 or HIFPH3 protein expressed in and purified from E coli in a conventional manner, or an oligopeptide having a part of the amino acid sequence of Lengsin, BJ-TSA-9, C20orf42, BUB1, C10orf3 or HIFPH3 synthesized in a conventional manner, and recovering the antibody from serum of the immunized animal in a conventional manner. When the antibody is monoclonal, it can be obtained by immunizing a non-human animal such as mouse with Lengsin, BJ-TSA-9, C20orf42, BUB1, C10orf3 or HIFPH3 protein expressed in and purified from E coli in a conventional manner, or an oligopeptide having a part of the amino acid sequence of any of those proteins synthesized in a conventional manner, subjecting the resultant splenocyte to cell fusion with a myeloma cell to prepare a hybridoma cell, and recovering the antibody from the hybridoma cell (Current protocols in Molecular Biology edit. Ausubel et al. (1987) Publish. John Wiley and Sons. Section 11.4-11.11).

The protein Lengsin, BJ-TSA-9, C20orf42, BUB1, C10orf3 or HIFPH3 used as an immunogen in the preparation of the antibody can be obtained from the sequence of any of SEQ ID NOS 1, 3, 5, 7, 9 and 11 provided herein, through procedures such as DNA cloning, construction of an corresponding plasmid, transfection of the plasmid into a host cell, culture of the resulting transformant, and recovery of the protein from culture of the transformant. These procedures can be conducted by any one of methods known to those skilled in the art or described in a literature (Molecular Cloning, T. Maniatis et al., CSH Laboratory (1983), DNA Cloning, D M. Glover, IRL PRESS (1985)), and the like.

Specifically, the protein used as an immunogen for preparation of the antibody of the invention can be obtained by constructing a recombinant DNA providing expression of Lengsin, BJ-TSA-9, C20orf42, BUB1, C10orf3 or HIFPH3 gene in a selected host cell (i.e., an expression vector), transfecting the DNA into a host cell to provide a transformant, culturing the transformant, and recovering the objective protein from culture of the transformant. Also, a partial peptide of Lengsin, BJ-TSA-9, C20orf42, BUB1, C10orf3 or HIFPH3 can be prepared by a usual method for chemical (peptide) synthesis based on the amino acid sequence provided herein (SEQ ID NO: 2, 4, 6, 8, 10 or 12).

Here, Lengsin, BJ-TSA-9, C20orf42, BUB1, C10orf3 or HIFPH3 of the invention includes not only a protein of an amino acid sequence of SEQ ID NO: 2, 4, 6, 8, 10 or 12 but also a homolog thereof. The homolog includes a protein consisting of an amino acid sequence which is the same as the amino acid sequence of SEQ ID NO: 2, 4, 6, 8, 10 or 12 except that one or more amino acids are deleted, substituted and/or added, and having an immunological activity equivalent to that known for the protein.

The homolog having an immunological activity equivalent to that known for the protein is, for example, a protein capable of inducing a specific immunoreaction in an appropriate animal or cells thereof and specifically binding to an antibody against Lengsin, BJ-TSA-9, C20orf42, BUB1, C10orf3 or HIFPH3.

There is no limitation regarding the number or position of the amino acid mutation in the protein as far as the immunological activity of the protein is maintained. Criteria based on which one can determine the number or position of the amino acid residue to be deleted, substituted and/or added without reducing the immunological activity can be obtained using a computer program well known in the art, such as DNA Star software. For example, the number of mutation would typically be within 10%, preferably 5%, more preferably 1% of the total amino acid residues. The amino acid to be substituted is not limited as far as the resulting protein maintains an immunological activity equivalent to Lengsin, BJ-TSA-9, C20orf42, BUB1, C10orf3 or HIFPH3. The amino acid introduced by substitution preferably has similar characteristics to that to be substituted in view of retention of structure, wherein the characteristics include polarity, charge, solubility, hydrophobicity, hydrophilicity, amphipathicity, and the like. For instance, Ala, Val, Leu, Ile, Pro, Met, Phe and Trp are classified into nonpolar amino acids; Gly, Ser, Thr, Cys, Tyr, Asn and Gln into non-charged amino acids; Asp and Glu into acidic amino acids; and Lys, Arg and His into basic amino acids. One of ordinary skill in the art can select an appropriate amino acid(s) falling within the same group on the basis of these criteria.

The antibody of the present invention may be obtained by using an oligopeptide having a part of the amino acid sequence of Lengsin, BJ-TSA-9, C20orf42, BUB1, C10orf3 or HIFPH3. The oligopeptide for the preparation of an antibody is not necessary to possess a functional biological activity, but it is desired to possess a similar immunogenicity to the corresponding protein. Preferably, the oligopeptide has such an immunogenicity and consists of at least 8, preferably 15, more preferably 20 contiguous amino acids from the amino acid sequence of BJ-TSA-9, C20orf42, BUB1, C10orf3 or HIFPH3.

To prepare the antibody against the oligopeptide, various adjuvants may be administered to a host simultaneously with the immunogen so as to enhance the immunoreactivity. Examples of the adjuvants are, but not limited to, Freund adjuvants; mineral gels such as aluminium hydroxide; surfactants such as lysolecithin, Pluronic® polyol, polyanion, peptide, oil emulsion, keyhole limpet hemocyanin and dinitrophenol; human adjuvants such as BCG (Bacille de Calmette-Guerin) or Corynebacterium.

In addition, commercially available antibodies including anti-Bub1 antibody (Upstate), anti-C10orf3 antibody (Abnova), anti-C20orf42 antibody (Imgenex), and anti-HIFPH3 antibody (Bethyl) can be used in the present invention.

The antibody of the invention has a characteristic of specifically binding to Lengsin, BJ-TSA-9, C20orf42, BUB1, C10orf3 or HIFPH3, and therefore can specifically detect the protein (and homologs thereof) expressed in a tissue of a test subject. Thus, the antibody of the invention is useful as a probe for detecting presence or absence of expression, or expression level, of Lengsin, BJ-TSA-9, C20orf42, BUB1, C10orf3 or HIFPH3 in a tissue of a test subject.

Specifically, Lengsin, BJ-TSA-9, C20orf42, BUB1, C10orf3 or HIFPH3 can be detected by obtaining a biological sample of a target tissue of a patient by biopsy and the like, preparing a tissue extract or proteins therefrom in a conventional manner, and conducting detection by a known method such as Western blotting, ELISA, or the like using the antibody as a probe in a conventional manner.

Diagnosis of cancer may be provided by examining the level of at least one of Lengsin, BJ-TSA-9, C20orf42, BUB1, C10orf3 and HIFPH3 in a tissue of a test subject compared to that of a healthy subject. Difference of the protein level may be presence or absence of the protein, or may be at least 2-fold, preferably 3-fold.

The antibody specifically recognizing Lengsin is particularly useful as a disease marker for lung adenocarcinoma, lung squamous cell carcinoma or gastric cancer.

The antibody specifically recognizing BJ-TSA-9 is particularly useful as a disease marker for leukemia, lung adenocarcinoma, lung squamous cell carcinoma, small cell lung cancer, oral cancer, gastric cancer, pancreas cancer or lymphoma.

The antibody specifically recognizing C20orf42 is particularly useful as a disease marker for lung squamous cell carcinoma, lung adenocarcinoma, liver cancer, gastric cancer, leukemia, malignant lymphoma tissues, rectal cancer, colon cancer or pancreas cancer.

The antibody specifically recognizing BUB1 is particularly useful as a disease marker for breast cancer, lung adenocarcinoma, lung squamous cell carcinoma, ovarian cancer, oral squamous cell carcinoma, renal cancer, large bowel cancer (colon cancer, rectal cancer), gastric cancer, pancreas cancer, liver cancer, leukemia, lymphoma or melanoma.

The antibody specifically recognizing C10orf3 is particularly useful as a disease marker for breast cancer, colon cancer, rectal cancer, renal cancer, gastric cancer, ovarian cancer, liver cancer, pancreas cancer, lung squamous cell carcinoma, lung adenocarcinoma, small cell lung cancer or melanoma.

The antibody specifically recognizing HIFPH3 is particularly useful as a disease marker for breast cancer, colon cancer, gastric cancer, renal cancer, pancreas cancer, liver cancer, lung adenocarcinoma or lung squamous cell carcinoma.

### (2) Method for Detecting (or Diagnosing) Cancer

The present invention provides a method for detecting (or diagnosing) cancer which utilizes the disease marker of the invention as mentioned above.

Specifically, the detection method of the present invention comprises collecting a biological sample of a target tissue from a test subject by biopsy and the like, detecting and measuring the expression level (amount) of Lengsin, BJ-TSA-9, C20orf42, BUB1, C10orf3 or HIFPH3 gene, or that of a protein encoded by the gene, and detecting (or diagnosing) presence or absence of, or severity of, cancer. The detection (diagnosis) method of the present invention can be used to detect (or diagnose) whether or not a therapeutic agent administered to a cancer patient for amelioration of cancer can actually provide improvement of the disease, and/or how much improvement the therapeutic agent can provide.

The method for detecting cancer of the present invention comprises the following steps (a), (b) and (c):

(a) bringing a biological sample prepared from a test subject into contact with the disease marker of the invention;

(b) measuring the expression level of Lengsin, BJ-TSA-9, C20orf42, BUB1, C10orf3 or HIFPH3 gene in the sample by using the disease marker as an indicator, and

(c) determining whether or not the test subject has cancer based on the result of (b).

The biological sample herein used may be a sample prepared from a target tissue of a test subject. Specifically, the biological sample may be a sample containing RNA prepared from the (target) tissue, a sample containing polynucleotides prepared from the RNA, or a sample containing proteins prepared from the target tissue. Such a sample containing RNA, polynucleotides, or proteins can be prepared by a conventional method after a part of a tissue of a test subject is collected by biopsy and the like

The diagnosis method of the present invention can be conducted as illustrated below depending on the biological sample to be examined.

### (2-1) When the Biological Sample to be Examined is RNA

When the biological sample to be examined is RNA, detection of cancer can be conducted by a method comprising the following steps (a), (b) and (c):

(a) hybridizing the disease marker(s) of the present invention (i.e., a polynucleotide having at least 15 contiguous nucleotides from the base sequence of Lengsin, BJ-TSA-9, C20orf42, BUB1, C10orf3 or HIFPH3 gene and/or a complementary polynucleotide thereof) with RNA prepared from a biological sample of a test subject or complementary polynucleotides transcribed therefrom;

b) detecting RNA prepared from the biological sample or complementary polynucleotides transcribed therefrom hybridized with the disease marker by using the disease marker as an indicator; and

(c) determining whether or not the test subject has cancer based on the result of the detection in (b).

When RNA is to be examined, the detection (diagnosis) method of the invention can be achieved by detecting and measuring the expression level of Lengsin, BJ-TSA-9, C20orf42, BUB1, C10orf3 or HIFPH3 gene in the RNA. Specifically, the method can be carried out by a known method such as Northern blotting, RT-PCR, DNA chip analysis, in situ hybridization, or the like by using the disease marker(s) of the invention (i.e., a polynucleotide comprising at least 15 contiguous nucleotides from the base sequence of Lengsin, BJ-TSA-9, C20orf42, BUB1, C10orf3 or HIFPH3 gene and/or a polynucleotide complementary thereto) as a primer or probe.

In the case of Northern blotting, the disease marker of the invention is used as a probe, and allows detection and measurement of presence or absence of expression, or expression level, of Lengsin, BJ-TSA-9, C20orf42, BUB1, C10orf3 or HIFPH3 gene in RNA. For example, the disease marker of the invention (which is a complementary strand) is labeled with a radioisotope (32P, 33P, etc.: RI) or a fluorescent substance. Then, the labeled disease marker is hybridized with RNA prepared from a tissue of a test subject which is transferred onto a nylon membrane or the like in a conventional manner. After that, a duplex formed between the labeled disease marker (DNA) and the RNA may be detected by detecting and measuring the signal from the label (RI or fluorescent substance) with a radiation detector (BAS-1800II, Fuji Photo Film., Inc.) or a fluorescence detector. Alternatively, the disease marker (i.e., a probe DNA) may be labeled with AlkPhos Direct Labelling and Detection System (Amersham Pharmacia Biotech) in accordance with the manufacturer's protocol and hybridized with RNA prepared from a tissue of a test subject, and then the signal from the label on the disease marker may be detected and measured using Multi-biomeasure STORM860 (Amersham Pharmacia Biotech).

In the case of RT-PCR, the disease marker of the invention is used as a primer, and presence or absence of expression, or expression level, of Lengsin, BJ-TSA-9, C20orf42, BUB1, C10orf3 or HIFPH3 gene in RNA can be detected and measured. For example, cDNA is prepared from RNA derived from a tissue of a test subject in a conventional manner and the cDNA is hybridized with a pair of primers (a forward primer hybridizing to the above-mentioned cDNA being a reverse strand, and a reverse primer binding to the forward strand) prepared from the disease marker of the present invention in order to amplify a target region corresponding to Lengsin, BJ-TSA-9, C20orf42, BUB1, C10orf3 or HIFPH3 gene using the cDNA as a template. Then, PCR is conducted in a conventional manner and the resulting amplified DNA duplex is detected. The detection of the amplified DNA duplex can be achieved by, for example, a method wherein the PCR is conducted with a primer previously labeled with RI or a fluorescent substance and the resulting labeled DNA duplex is detected; a method wherein the resulting DNA duplex is transferred onto a nylon membrane or the like, and is then detected due to hybridization with a labeled disease marker that serves as a probe. The resulting labeled DNA duplex products can be measured using Agilent 2100 Bioanalyser (Yokogawa Analytical Systems Inc.) or the like. The measurement may also be achieved by preparing RT-PCR reaction solution using SYBR Green RT-PCR Reagents (Applied Biosystems) according to the manufacture's protocol, conducting the reaction using ABI PRISM 7700 Sequence Detection System (Applied Biosystems), and detecting the reaction products.

In the case of DNA chip analysis, for example, a DNA chip on which the disease marker of the invention is attached is prepared as a DNA probe (which is either single- or double-stranded), and the DNA chip is hybridized with cRNA prepared from RNA derived from a tissue of a test subject in a conventional manner. Then, a duplex formed by DNA and cRNA is detected by binding a labeled probe prepared from the disease marker of the invention to the duplex. A DNA chip capable of detecting and measuring the expression level of Lengsin, BJ-TSA-9, C20orf42, BUB1, C10orf3 or HIFPH3 gene may be used for the present invention. An example of such a DNA chip is Gene Chip Human Genome U95 A, B, C, D, E (Affymetrix). Detection and measurement of the expression level of Lengsin, BJ-TSA-9, C20orf42, BUB1, C10orf3 or HIFPH3 gene in RNA of a test subject using such a DNA chip is described in Examples below.

### (2-2) When the Biological Sample to be Examined is Proteins

When the biological sample to be examined is proteins, the detection (diagnosis) method of the invention is conducted by detecting and measuring the level (amount) of Lengsin, BJ-TSA-9, C20orf42, BUB1, C10orf3 or HIFPH3 in a biological sample. Specifically, the method may be comprises the following steps (a), (b) and (c):

(a) allowing proteins prepared from a biological sample of a test subject to bind to the disease marker of the present invention being an antibody (i.e., the antibody recognizing Lengsin, BJ-TSA-9, C20orf42, BUB1, C10orf3 or HIFPH3);

b) detecting proteins prepared from the biological sample or partial peptides derived therefrom bound to the disease marker by using the disease marker as an indicator; and

(c) determining whether or not the test subject has cancer based on the result of the detection in (b).

Specifically, the method may be a known method such as Western blotting for detecting and measuring Lengsin, BJ-TSA-9, C20orf42, BUB1, C10orf3 or HIFPH3 by using the disease marker of the invention being an antibody (i.e., the antibody specifically recognizing Lengsin, BJ-TSA-9, C20orf42, BUB1, C10orf3 or HIFPH3).

Western blotting can be carried out by using the antibody, that is, the disease marker of the invention, as the first antibody and, as the second antibody which binds to the first antibody, an antibody labeled with a radio isotope such as ¹²⁵I, fluorescent substance, or the like, and detecting and measuring the signal from the radio isotope or fluorescent substance in a resulting labeled complex using a radiation detector (BAS-1800II, Fuji Photo Film., Inc., etc), a fluorescence detector, or the like. Alternatively, after using the disease marker of the invention as the first antibody, detection and measurement may be carried out with ECL Plus Western Blotting Detection System (Amersham Pharmacia Biotech) according to the manufacture's protocol, and with Multi-biomeasure STORM860 (Amersham Pharmacia Biotech).

### (2-3) Diagnosis of Cancer

Diagnosis of cancer can be carried out by comparing the expression level of Lengsin, BJ-TSA-9, C20orf42, BUB1, C10orf3 or HIFPH3 gene, or the expression level (or amount) of Lengsin, BJ-TSA-9, C20orf42, BUB1, C10orf3 or HIFPH3 protein in a tissue of a test subject with that in a normal tissue and assessing the difference between the two.

A biological sample (RNA or protein) collected or prepared from a normal tissue to be required can be obtained from a tissue of a subject without cancer or from a non-cancerous, normal tissue of a cancer patient, by biopsy or by collecting a postmortem tissue with his consent. The “subject without cancer” means a subject being at least asymptomatic, preferably not diagnosed as cancer as a result of any other diagnosis method such as X-ray examination. The “subject without cancer” may be herein referred to as a “healthy subject” simply.

Comparison of the expression level of the gene or protein in a tissue of a test subject with that in a normal tissue (i.e., a tissue of a subject without cancer or a non-cancerous, normal tissue of a cancer patient) can be carried out by measuring in parallel biological samples of both test and healthy subjects. The expression level of the gene or protein in a healthy subject is not necessarily that measured in parallel and, an average value or a statistical median value of the expression level of Lengsin, BJ-TSA-9, C20orf42, BUB1, C10orf3 or HIFPH3 gene or protein which is determined by a measurement of plural (at least 2, preferably equal to or more than 3, more preferably equal to or more than 5) normal tissue samples under a constant condition may be used.

The test subject is determined as having cancer when the expression level of Lengsin, BJ-TSA-9, C20orf42, BUB1, C10orf3 or HIFPH3 gene or the expression product thereof, that is, Lengsin, BJ-TSA-9, C20orf42, BUB1, C10orf3 or HIFPH3 protein in the tissue of the test subject is 2-fold, preferably 3-fold higher than that of a healthy subject. The test subject is determined as having cancer or suspected to have cancer when the expression level of the gene or protein in the test subject is higher than that in a healthy subject.

Specifically, the test subject is suspected to have cancer when the expression level of Lengsin gene or protein, among the genes and proteins above, is higher in lung or stomach of the test subject than that in a corresponding normal tissue.

Also, the test subject is suspected to have cancer when the expression level of BJ-TSA-9 gene or protein, is higher in leukocytes, lung, oral cavity, stomach, pancreas or lymph node of the test subject than that in a corresponding normal tissue.

Also, the test subject is suspected to have cancer when the expression level of C20orf42 gene or protein, is higher in lung, liver, stomach, leukocyte, lymph node, rectum, colon or pancreas of the test subject than that in a corresponding normal tissue.

Also, the test subject is suspected to have cancer when the expression level of BUB1 gene or protein, is higher in mammary gland, lung, ovary, oral cavity, kidney, large intestine (colon, rectum), stomach, pancreas, liver, leukocyte, lymph node or skin of the test subject than that in a corresponding normal tissue.

Also, the test subject is suspected to have cancer when the expression level of C10orf3 gene or protein, is higher in mammary gland, colon, rectum, kidney, stomach, ovary, liver, pancreas, lung or skin of the test subject than that in a corresponding normal tissue.

Also, the test subject is suspected to have cancer when the expression level of HIFPH3 gene or protein, is higher in mammary gland, colon, stomach, kidney, pancreas, liver, lung of the test subject than that in a corresponding normal tissue.

The present invention is specifically explained by the following examples, but the examples should not be deemed to limit the present invention in any sense.

### Example 1

### Preparation of Total RNA from Human Tissue Samples and Human Cancer cell lines

Total RNA was prepared by a conventional method from samples of cancer and normal tissues of human major organs as follows: lung adenocarcinoma (57 samples), normal lung (derived from lung adenocarcinoma patients) (46 samples), lung squamous cell carcinoma (48 samples), normal lung (derived from lung squamous cell carcinoma patients) (18 samples), colon cancer (108 samples), normal colon (derived from colon cancer patients) (114 samples), rectal cancer (43 samples), normal rectum (derived from rectal cancer patients) (31 samples), gastric cancer (38 samples), normal stomach (derived from gastric cancer patients) (13 samples), breast cancer (237 samples), normal breast (derived from breast cancer patients) (39 samples), ovarian cancer (37 samples), normal ovarian (derived from ovarian cancer patients) (5 samples), liver cancer (19 samples), normal liver (derived from liver cancer patients) (8 samples), renal cancer (89 samples), normal kidney (derived from renal cancer patients) (64 samples), pancreas cancer (55 samples), normal pancreas (derived from pancreas cancer patients) (16 samples), leukemia (6 samples), lymphoma (90 samples), normal bone marrow (2 samples), melanoma (10 samples), and normal skin (72 samples).

### Example 2

### DNA Chip Analysis

Using the total RNA prepared from the samples as described in Example 1, DNA chip analysis was performed. For the DNA chip analysis, Gene Chip Human Genome U133 set (Affymetrix) was used. Specifically, the analysis comprised the following steps: (1) preparation of cDNA from the total RNA, (2) preparation of labeled cRNA from the cDNA, (3) fragmentation of the labeled cRNA, (4) hybridization of the fragmented cRNA with a probe array, (5) staining of the probe array, (6) scanning of the probe array, and (7) analysis of gene expression.

(1) Preparation of cDNA from Total RNA

A mixture containing each total RNA (10 μg) prepared in Example 1 and T7-(dT)24 primer (Amersham) (100 pmol) (11 μl) was heated at 70° C. for 10 min and cooled on ice. After the cooling, 5× First Strand cDNA Buffer (4 μL), 0.1 M DTT (dithiothreitol) (2 μl), and 10 mM dNTP Mix (11) (SuperScript Choice System for cDNA Synthesis (Gibco-BRL)) were added and the mixture was heated at 42° C. for 2 min. Then, Super ScriptII RT (2 μl, 400U) (included in the kit as mentioned above) was added and the mixture was heated at 42° C. for 1 h and then cooled on ice. After the cooling, sterile distilled water treated with DEPC (nacalai tesque) (91 μl) and 5× Second Strand Reaction Buffer (30 μL), 10 mM dNTP Mix (3 μl), E. coli DNA Ligase (1 μl, 10U), E. Coli DNA Polymerase I (4 μl, 40U) and E. coli RNaseH (1 μl, 2U) (included in the kit as mentioned above) were added, and the mixture was incubated at 16° C. for 2 h. After T4 DNA Polymerase (2 μl, 10U) (included in the kit as mentioned above) was added, the mixture was further incubated at 16° C. for 5 min, and then added with 0.5M EDTA (10 μl). Phenol/chloroform/isoamyl alcohol solution (NIPPON GENE CO., LTD.) (162 μl) was added and mixed. The mixture was moved to Phase Lock Gel Light (Eppendorf) previously centrifuged at room temperature, at 14,000 rpm for 30 seconds, and centrifuged at room temperature, at 14,000 rpm for 2 min, and its aqueous phase (145 μl) was moved to an Eppendorf tube. The obtained solution was added with 7.5M ammonium acetate solution (72.5 μl) and ethanol (362.5 μl) and mixed, and then centrifuged at 4° C., at 14,000 rpm for 20 min. After the centrifugation, a pellet containing cDNA prepared was obtained by discarding the supernatant. Then, the pellet was added with 80% ethanol (0.5 mL) and centrifuged at 4° C., at 14,000 rpm for 5 min and the supernatant was discarded. After the same procedure was repeated, the pellet was dried, and lysed with DEPC-treated water (12 μl). As a result, cDNA was obtained from each total RNA prepared in Example 1.

(2) Preparation of Labeled cRNA from cDNA

Each cDNA solution (5 μl) was mixed with DEPC-treated water (17 μl) and 10×HY Reaction Buffer (4 μl), 10× Biotin Labeled Ribonucleotides (4 μl), 10×DTT (4 μl), 10× RNase Inhibitor Mix (4 μl) and 20×T7 RNA Polymerase (2 μl) (BioArray High Yield RNA Transcript Labeling Kit (ENZO)), and incubated at 37° C. for 5 h to prepare labeled cRNA. After the incubation, DEPC-treated water (60 μl) was added to the solution, and the labeled cRNA prepared was purified by RNeasy Mini Kit (GIAGEN) according to the manufacture's protocol.

(3) Fragmentation of Labeled cRNA

A solution containing each labeled cRNA (20 μg) was added with 5× Fragmentation Buffer (200 mM Tris-acetic acid pH 8.1 (Sigma), 500 mM potassium acetate (Sigma), 150 mM magnesium acetate (Sigma)) (8 μl), and the solution (401) was heated at 94° C. for 35 min and placed on ice. As a result, fragmentation of the labeled cRNA was achieved.

(4) Hybridization of Fragmented cRNA with a Probe Array

Each fragmented cRNA (40 μl) was added with 5 nM Control Oligo B2 (Amersham) (4 μl), 100× Control cRNA Cocktail (4 μl), Herring sperm DNA (Promega) (40 μg), Acetylated BSA (Gibco-BRL) (200 μg), 2×MES Hybridization Buffer (200 mM MES, 2M [Na⁺], 40 mM EDTA, 0.02% Tween20 (Pierce), pH 6.5-6.7) (200 μl) and DEPC-treated water (144 μl) to yield a hybridization cocktail (4001). Each hybridization cocktail obtained was heated at 99° C. for 5 min and then 45° C. for 5 min. After heated, the hybridization cocktail was centrifuged at room temperature, at 14,000 rpm for 5 min to obtain the supernatant.

In the meantime, a prove array was prepared by filling Human genome U133 pribe array (Affymetrix) with 1×MES hybridization buffer, rotating the array in a hybridization oven at 45° C., 60 rpm for 10 min and then removing the 1×MES hybridization buffer. The supernatant of each hybridization cocktail obtained as above (200 μl) was added to the probe array, and the probe array was rotated in a hybridization oven at 45° C., 60 rpm for 16 h to hybridize the fragmented cRNA to the probe array.

### (5) Staining of a Probe Array

After the hybridization cocktail was removed from each probe array hybridized with the fragmented cRNA, the probe array was filled with Non-Stringent Wash Buffer (6×SSPE (diluted from 20×SSPE (nacalai tesque)), 0.01% Tween20 and 0.005% Antifoam0-30 (Sigma). Then, the probe array hybridized with the fragmented cRNA was placed to a given position in GeneChip Fluidics Station 400 (Affymetrix) set with Non-Stringent Wash Buffer and Stringent Wash Buffer (100 mM MES, 0.1 M NaCl, 0.01% Tween20). After that, according to a staining protocol EuKGE-WS2, the prove array was stained with the first staining solution (10 μg/mL Streptavidin Phycoerythrin (SAPE) (MolecuLar Probe), 2 mg/mL Acetylated BSA, 100 mM MES, 1 M NaCl (Ambion), 0.05% Tween20, 0.005% Antifoam0-30), and with the second staining solution (100 μg/mL Goat IgG (Sigma), 3 μg/mL Biotinylated Anti-Streptavidin antibody (Vector Laboratories), 2 mg/mL Acetylated BSA, 100 mM MES, 1 M NaCl, 0.05% Tween20, 0.005% Antifoam0-30).

### (6) Scanning of a Probe Array and (7) Analysis of Gene Expression Level

The stained probe array was applied to HP GeneArray Scanner (Affymetrix) and the staining pattern was read. Based on the staining pattern, gene expression on the probe array was analyzed by GeneChip Workstation System (Affymetrix). Then, after normalization according to an analysis protocol, expression level (average difference), and presence or absence of expression, of each probe (i.e., each gene) in each sample were calculated. For each probe, an average value of gene expression level was calculated for each of different kinds of samples, and differences in the expression level and frequency between the different kinds of samples was obtained.

### Example 3

### Analysis of Variation in Expression

As described below, genes showing increase of expression level and/or frequency in cancer tissues compared to normal tissues originated from various major organs (lung, colon, rectum, stomach, breast, liver, kidney, ovary, pancreas) were selected.

According to the result of the DNA chip analysis for gene expression, from a set of probes whose expression level and/or frequency were increased in cancer tissues compared to the corresponding normal tissues, probes showing specific increase of expression level and/or frequency in many cancer tissues were selected. Then, genes corresponding to those selected probes were checked and selected.

As a result, six genes, BUB1, C10orf3, C20orf42, Lengsin, HIFPH3 and BJ-TSA-9 genes, were selected. Ratios of variation in expression of those six genes are described in Table 2 and expression frequencies are in Table 3.

TABLE 2

| | | | | | | Lung | | | | | | | |
|---|---|---|---|---|---|---|---|---|---|---|---|---|---|
| | | | | | Lung | squamous | | | | | | | |
| | Breast | Colon | Renal | Liver | adeno- | cell | Pancreas | Gastric | Ovarian | Rectal | | Malignant | |
| Gene | cancer | cancer | cancer | cancer | carcinoma | carcinoma | cancer | cancer | cancer | cancer | Leukemia | lymphoma | Melanoma |
| BUB1 | 2.1 | 2.0 | 2.6 | 3.1 | 2.0 | 4.0 | 2.0 | 2.4 | 4.2 | 2.3 | 1.4 | 1.3 | 3.1 |
| C10orf3 | 2.5 | 2.3 | 2.9 | 6.7 | 3.0 | 6.2 | 2.4 | 2.4 | 9.0 | 2.9 | 0.9 | 0.9 | 3.9 |
| C20orf42 | 0.5 | 2.8 | 0.4 | 2.0 | 6.0 | 15.4 | 4.2 | 2.4 | 0.3 | 2.4 | 2.0 | 1.3 | 0.4 |
| Lengsin | 1.0 | 1.0 | 0.4 | 0.7 | 8.5 | 2.9 | 2.1 | 2.3 | 1.9 | 1.2 | 1.3 | 1.8 | 0.9 |
| HIFPH3 | 2.3 | 1.3 | 15.5 | 2.6 | 2.3 | 15.9 | 4.4 | 1.2 | 0.9 | 0.9 | 0.6 | 0.6 | 0.2 |
| BJ-TSA-9 | 1.0 | 0.8 | 1.0 | 1.5 | 9.4 | 8.1 | 2.6 | 3.6 | 4.3 | 0.9 | 14.9 | 2.3 | 0.5 |

TABLE 3

| | | | | | | | | | Lung | |
|---|---|---|---|---|---|---|---|---|---|---|
| | Breast | Normal | Colon | Normal | | | | | adeno- | |
| Gene | cancer | breast | cancer | colon | Renal cancer | Normal kidney | Liver cancer | Normal liver | carcinoma | Normal lung |
| BUB1 | 39 | 8 | 69 | 21 | 8 | 0 | 26 | 0 | 39 | 11 |
| C10orf3 | 62 | 15 | 89 | 54 | 15 | 0 | 21 | 0 | 60 | 13 |
| C20orf42 | 2 | 3 | 97 | 66 | 0 | 0 | 0 | 0 | 18 | 0 |
| Lengsin | 0 | 0 | 0 | 1 | 0 | 17 | 11 | 13 | 53 | 0 |
| HIFPH3 | 14 | 5 | 35 | 25 | 78 | 2 | 11 | 0 | 25 | 7 |
| BJ-TSA-9 | 2 | 3 | 0 | 1 | 0 | 0 | 0 | 0 | 39 | 0 |

| | Lung | | | | | | | | | |
|---|---|---|---|---|---|---|---|---|---|---|
| | squamous cell | | Pancreas | | Gastric | Normal | Ovarian | Normal | Rectal | Normal |
| Gene | carcinoma | Normal lung | cancer | Normal pancreas | cancer | stomach | cancer | ovarian | cancer | rectum |
| BUB1 | 63 | 11 | 22 | 6 | 61 | 23 | 70 | 0 | 74 | 13 |
| C10orf3 | 79 | 6 | 56 | 6 | 89 | 46 | 84 | 20 | 84 | 48 |
| C20orf42 | 48 | 0 | 53 | 0 | 68 | 38 | 3 | 20 | 95 | 81 |
| Lengsin | 8 | 0 | 0 | 0 | 13 | 0 | 5 | 0 | 5 | 0 |
| HIFPH3 | 52 | 0 | 25 | 0 | 16 | 8 | 27 | 20 | 26 | 32 |
| BJ-TSA-9 | 23 | 0 | 4 | 0 | 3 | 0 | 5 | 0 | 0 | 0 |

| | | | Normal | | |
|---|---|---|---|---|---|
| | | Malignant | bone | | |
| Gene | Leukemia | lymphoma | marrow | Melanoma | Normal skin |
| BUB1 | 100 | 76 | 100 | 70 | 3 |
| C10orf3 | 83 | 81 | 100 | 50 | 7 |
| C20orf42 | 0 | 1 | 0 | 10 | 24 |
| Lengsin | 0 | 0 | 0 | 0 | 0 |
| HIFPH3 | 17 | 0 | 0 | 0 | 63 |
| BJ-TSA-9 | 17 | 0 | 0 | 0 | 1 |

Values in Table 2 indicate ratio of variation in expression in various cancer tissues compared to the corresponding normal tissues, wherein the expression level in each cancer tissue was defined in relation to the expression level in each normal tissue analyzed with Human Genome U133 Chip regarded as 1. Values in Table 3 indicate expression frequencies (%) of the genes in various cancer and normal tissues.

BUB1 gene showed increase of expression level in all cancer tissues examined compared to normal tissues and the increase was particularly significant in lung squamous cell carcinoma and ovarian cancer. Expression frequency was also increased, and the increase was particularly significant in lung squamous cell carcinoma, ovarian cancer and melanoma tissues.

C10orf3 gene showed increase of expression level in breast cancer, colon cancer, renal cancer, liver cancer, lung adenocarcinoma, lung squamous cell carcinoma, pancreas cancer, gastric cancer, ovarian cancer, rectal cancer and melanoma tissues compared to normal tissues and the increase was particularly significant in ovarian cancer, liver cancer and lung squamous cell carcinoma. Expression frequency was also increased in all cancers except for blood cancer as observed in the expression level, and the increase was particularly significant in lung squamous cell carcinoma, pancreas cancer and ovarian cancer.

C20orf42 gene showed increase of expression level in colon cancer, liver cancer, lung adenocarcinoma, lung squamous cell carcinoma, pancreas cancer, gastric cancer, rectal cancer, leukemia and malignant lymphoma tissues compared to normal tissues, and the increase was particularly significant in lung squamous cell carcinoma and lung adenocarcinoma. Expression frequency was also increased and the increase was particularly significant in lung adenocarcinoma, lung squamous cell carcinoma and pancreas cancer.

Lengsin gene showed increase of expression level in lung adenocarcinoma, lung squamous cell carcinoma and gastric cancer compared to normal tissues, and the increase was particularly significant in lung adenocarcinoma. Expression frequency was also increased in lung adenocarcinoma, lung squamous cell carcinoma and gastric cancer as observed in the expression level, and the increase was particularly significant in lung adenocarcinoma.

HIFPH3 gene showed increase of expression level in tissues of breast cancer, colon cancer, renal cancer, liver cancer, lung adenocarcinoma, lung squamous cell carcinoma, pancreas cancer and gastric cancer compared to normal tissues, and the increase was particularly significant in renal cancer and lung squamous cell carcinoma. Expression frequency was also increased in breast cancer, colon cancer, renal cancer, liver cancer, lung adenocarcinoma, lung squamous cell carcinoma, pancreas cancer and gastric cancer as observed in the expression level, and the increase was particularly significant in renal cancer, lung squamous cell carcinoma and pancreas cancer.

BJ-TSA-9 gene showed increase of expression level in lung adenocarcinoma, lung squamous cell carcinoma and leukemia compared to normal tissues, and the increase was particularly significant in leukemia, lung adenocarcinoma and lung squamous cell carcinoma. Expression frequency was also increased in lung adenocarcinoma, lung squamous cell carcinoma and leukemia as observed in the expression level, and the increase was particularly significant in lung adenocarcinoma and lung squamous cell carcinoma.

AS shown above, the selected six genes showed increase of expression level and frequency specifically in cancer tissues compared to the corresponding normal tissues. Accordingly, those six genes and expression products thereof (i.e., proteins) were found to be useful as disease markers for cancers (especially for cancers as mentioned above).

### Example 4

### Expression Analysis of Tumor Antigen Genes in Different Cells and Tissues by RT-PCR Method

Expression of the above six genes in different cancer cell lines, cancer tissues, and normal tissues were analyzed by RT-PCR method. cDNAs were prepared with oligo dT primer from RNAs extracted from different cancer cell lines and cancer tissues using Isogen reagent (Nippon Gene) or RNAs derived from normal tissues which were commercially available (clontech), and amplified by PCR reactions (40 cycles) using the combinations of primers described in Table 4 (BUB1 gene, primer 1: SEQ ID NO: 208, primer 2: SEQ ID NO: 209; C10orf3 gene, primer 1: SEQ ID NO: 210, primer 2: SEQ ID NO: 211; C20orf42 gene, primer 1: SEQ ID NO: 206, primer 2: SEQ ID NO: 207; Lengsin gene, primer 1: SEQ ID NO: 202, primer 2: SEQ ID NO: 203; HIFPH3 gene, primer 1: SEQ ID NO: 212, primer 2: SEQ ID NO: 213; BJ-TSA-9 gene, primer 1: SEQ ID NO: 204, primer 2: SEQ ID NO: 205). After that, the amplified cDNAs were separated by electrophoresis to be analyzed. As a positive control, expression of G3PDH gene was confirmed by RT-PCR method in the same way as described above. A part of the cancer and normal tissues used were prepared after informed consent was obtained mainly from surgical samples. The results are shown in FIGS. 1 to 6.

TABLE 4

| Gene | Primer 1 | Primer 2 |
|---|---|---|
| lengsin | tggcactggaagaagatcaa | tcaccacaactttgttgttt |
| | | gtt |
| BJ-TSA-9 | cagatccaggtgcctctgac | tcaggtaatccaaccaccttg |
| C20orf42 | tgaatttctgaggccttgct | tgttctcagcagcaaacagg |
| BUB1 | ttatctgctggcttggcact | gcttttgccttaacaaatcca |
| C10orf3 | tgtccattgttaagaggtgg | tgagagggctacatgggttt |
| | tg | |
| HIFPH3 | catccctgtcttgtgtgtgg | ccaacagccctggattaaga |

BUB1 gene was not expressed, or expressed at a low level, in most of the normal tissues, but highly expressed in oral squamous cell carcinoma, renal cancer, large bowel cancer, gastric cancer, pancreas cancer, liver cancer, lymphoma and melanoma cell lines.

C10orf3 gene was not expressed, or expressed at a low level, in most of the normal tissues except for testis, but highly expressed in lung adenocarcinoma, lung squamous cell carcinoma and small cell lung cancer cell lines.

C20orf42 gene was not expressed in most of the normal tissues, but highly expressed in colon cancer and pancreas cancer cell lines.

Lengsin gene was not expressed in most of the normal tissues, but highly expressed in lung adenocarcinoma and lung squamous cell carcinoma cell lines.

HIFPH3 gene was expressed in most of the normal tissues, but expressed at a higher level in renal cancer cell lines. In addition, HIFPH3 gene was expressed in many of the cancer tissues originated from renal cancer patients at a higher level compared to the normal tissues.

BJ-TSA-9 was not expressed in most of the normal tissues, but highly expressed in lung adenocarcinoma, lung squamous cell carcinoma, small cell lung cancer, oral cancer, gastric cancer, pancreas cancer and lymphoma cell lines.

In summary, those six genes were highly expressed in various cancer cell lines and tissues, although the types of cancer were different from each other, but not expressed in normal tissues or expressed at a lower lever in normal tissues than in cancer cells or tissues. In addition to the results of the DNA chip analysis, those results strongly suggest that the six genes and expression products thereof (i.e., proteins) are useful as disease markers for cancers (especially for cancers as described above).

### Example 5

### Synthesis and Selection of Candidate Peptides

### (1) Selection of Candidate Peptides

From the amino acid sequence of Lengsin (SEQ ID NO: 2), peptides consisting of the amino acid sequences of SEQ ID NOS: 13 to 31 and 195 to 201 and those of SEQ ID NOS: 32 to 41 were selected as candidate peptides being potential to bind to HLA-A24 and HLA-A2 molecules, respectively.

From the amino acid sequence of BJ-TSA-9 (SEQ ID NO: 4), peptides consisting of the amino acid sequences of SEQ ID NOS: 42 to 49 and those of SEQ ID NOS: 50 to 58 were selected as candidate peptides being potential to bind to HLA-A24 and HLA-A2 molecules, respectively.

From the amino acid sequence of C20orf42 (SEQ ID NO: 6), peptides consisting of the amino acid sequences of SEQ ID NOS: 59 to 78 and those of SEQ ID NOS: 79 to 88 were selected as candidate peptides being potential to bind to HLA-A24 and HLA-A2 molecules, respectively.

From the amino acid sequence of BUB1 (SEQ ID NO: 8), peptides consisting of the amino acid sequences of SEQ ID NOS: 89 to 117 and those of SEQ ID NOS: 118 to 157 were selected as candidate peptides being potential to bind to HLA-A24 and HLA-A2 molecules, respectively.

From the amino acid sequence of C10orf3 (SEQ ID NO: 10), peptides consisting of the amino acid sequences of SEQ ID NOS: 158 to 165 and those of SEQ ID NOS: 166 to 175 were selected as candidate peptides being potential to bind to HLA-A24 and HLA-A2 molecules, respectively.

From the amino acid sequence of HIFPH3 (SEQ ID NO: 12), peptides consisting of the amino acid sequences of SEQ ID NOS: 176 to 183 and those of SEQ ID NOS: 184 to 194 were selected as candidate peptides being potential to bind to HLA-A24 and HLA-A2 molecules, respectively. The position on the amino acid sequence from which the peptide is derived as well as the length and the amino acid sequence of the peptide are listed in Tables 5 to 16 below.

TABLE 5
Lengsin A24 peptides

| Peptide | Amino Acid Sequence | SEQ ID NO |
|---|---|---|
| Lengsin_295(10) | KYNYIASFFI | 13 |
| Lengsin_368(9) | RYSKDRKDL | 14 |
| Lengsin_447(9) | FYQVEPSEI | 15 |
| Lengsin_489(9) | KYELENEEI | 16 |
| Lengsin_49(9) | DMSNSNDCM | 17 |
| Lengsin_56(9) | CMRDSSQIL | 18 |
| Lengsin_72(9) | RMKHIRQAM | 19 |
| Lengsin_86(10) | QFVRFEATDL | 20 |
| Lengsin_118(10) | CMPRGYLEVI | 21 |
| Lengsin_142(9) | CFNSDIVLM | 22 |
| Lengsin_160(10) | PWADRTARVI | 23 |
| Lengsin_172(10) | TFTVTGEPLL | 24 |
| Lengsin_199(9) | GFSLLSAFI | 25 |
| Lengsin_238(10) | PFMQELVDGL | 26 |
| Lengsin_265(10) | QMEISFLPEF | 27 |
| Lengsin_318(10) | LWDVDRKKNM | 28 |
| Lengsin_383(10) | TWGYNDNSCI | 29 |
| Lengsin_385(9) | GYNDNSCIF | 30 |
| Lengsin_479(9) | TFIRYFVAM | 31 |
| Lengsin_149(11) | LMPELSTFRVL | 195 |
| Lengsin_326(12) | NMFCSTSGTEQL | 196 |
| Lengsin_486(12) | AMKKYELENEEI | 197 |
| Lengsin_185(11) | RYIAKRQLSHL | 198 |
| Lengsin_247(11) | LYHTGANVESF | 199 |
| Lengsin_482(11) | RYFVAMKKYEL | 200 |
| Lengsin_207(12) | IYDFCIFGVPEI | 201 |

TABLE 6
Lengsin A2 peptides

| Peptide | Amino Acid Sequence | SEQ ID NO |
|---|---|---|
| Lengsin_313(9) | ILSHSLWDV | 32 |
| Lengsin_239(9) | FMQELVDGL | 33 |
| Lengsin_246(9) | GLYHTGANV | 34 |
| Lengsin_206(10) | FIYDFCIFGV | 35 |
| Lengsin_149(10) | LMPELSTFRV | 36 |
| Lengsin_79(10) | AMAKNRLQFV | 37 |
| Lengsin_312(10) | GILSHSLWDV | 38 |
| Lengsin_270(10) | FLPEFGISSA | 39 |
| Lengsin_231(10) | FLNNHDQPFM | 40 |
| Lengsin_336(10) | QLTITGKKWL | 41 |

TABLE 7
BJ-TSA-9 A24 peptides

| Peptide | Amino Acid Sequence | SEQ ID NO |
|---|---|---|
| BJ-TSA-9_20(10) | QWVRPARADF | 42 |
| BJ-TSA-9_120(10) | SWASAEKPYL | 43 |
| BJ-TSA-9_138(10) | YFQTVKHNNI | 44 |
| BJ-TSA-9_165(10) | LMDVFTDVEI | 45 |
| BJ-TSA-9_227(10) | IYCAKSGRKF | 46 |
| BJ-TSA-9_273(9) | KFTGQAVEL | 47 |
| BJ-TSA-9_281(9) | LFDEEFRHL | 48 |
| BJ-TSA-9_289(9) | LYASSKPVM | 49 |

TABLE 8
BJ-TSA-9 A2 peptides

| Peptide | Amino Acid Sequence | SEQ ID NO |
|---|---|---|
| BJ-TSA-9_164(9) | ILMDVFTDV | 50 |
| BJ-TSA-9_252(9) | VLSGSYSFT | 51 |
| BJ-TSA-9_100(9) | LQSGTYFPV | 52 |
| BJ-TSA-9_99(10) | SLQSGTYFPV | 53 |
| BJ-TSA-9_191(10) | VLLDQGGVKL | 54 |
| BJ-TSA-9_199(10) | KLFQEMCDKV | 55 |
| BJ-TSA-9_62(10) | FLSSVEAQYI | 56 |
| BJ-TSA-9_270(10) | ILSKFTGQAV | 57 |
| BJ-TSA-9_163(10) | VILMDVFTDV | 58 |

TABLE 9
C20orf42 A24 peptides

| Peptide | Amino Acid Sequence | SEQ ID NO |
|---|---|---|
| C20orf42_79(10) | KYGVQADAKL | 59 |
| C20orf42_182(9) | LYSKTMTPI | 60 |
| C20orf42_283(10) | KYDAVRINQL | 61 |
| C20orf42_292(10) | LYEQARWAIL | 62 |
| C20orf42_319(9) | QYHISKLSL | 63 |
| C20orf42_381(10) | SYQPEVLNIL | 64 |
| C20orf42_655(9) | EYIGGYIFL | 65 |
| C20orf42_52(9) | VMLKLVEQI | 66 |
| C20orf42_68(9) | CWLLKTHWTL | 67 |
| C20orf42_89(9) | LFTPQHKML | 68 |
| C20orf42_202(10) | TMTWFSDSPL | 69 |
| C20orf42_249(9) | GWLDSSRSL | 70 |
| C20orf42_271(10) | RFKYYSFFDL | 71 |
| C20orf42_392(10) | YWFIFKDTSI | 72 |
| C20orf42_460(10) | QYAQWMAACM | 73 |
| C20orf42_510(9) | NMDMNPECF | 74 |
| C20orf42_545(9) | QMPLVEAKL | 75 |
| C20orf42_554(9) | RFIQAWQSL | 76 |
| C20orf42_558(10) | AWQSLPEFGL | 77 |
| C20orf42_603(10) | TWRFTNIKQW | 78 |

TABLE 10
C20orf42 A2 peptides

| Peptide | Amino Acid Sequence | SEQ ID NO |
|---|---|---|
| C20orf42_69(9) | WLLKTHWTL | 79 |
| C20orf42_361(9) | SLLEDITDI | 80 |
| C20orf42_377(9) | KLFRPKKLL | 81 |
| C20orf42_417(9) | KLNLRGCEV | 82 |
| C20orf42_300(9) | ILLEEIDCT | 83 |
| C20orf42_610(9) | KQWNVNWET | 84 |
| C20orf42_621(9) | VVIEFDQNV | 85 |
| C20orf42_291(10) | QLYEQARWAI | 86 |
| C20orf42_95(10) | KMLRLRLPNL | 87 |
| C20orf42_88(10) | LLFTPQHKML | 88 |

TABLE 11
Bub1 A24 peptides

| Peptide | Amino Acid Sequence | SEQ ID NO |
|---|---|---|
| Bub1_43(10) | EYLITLLEHL | 89 |
| Bub1_61(9) | KYHNDPRFI | 90 |
| Bub1_89(9) | LYNHGIGTL | 91 |
| Bub1_101(9) | LYIAWAGHL | 92 |
| Bub1_139(9) | QYRLFQTRL | 93 |
| Bub1_670(9) | MYSASLLRL | 94 |
| Bub1_801(9) | VYEATQGDL | 95 |
| Bub1_870(9) | SYGTLLNAI | 96 |
| Bub1_1061(9) | HYTNKIRAL | 97 |
| Bub1_29(10) | RYIQWVEENF | 98 |
| Bub1_77(10) | EYNSDLHQFF | 99 |
| Bub1_206(9) | NMERRVITI | 100 |
| Bub1_235(9) | VMYCKEKLI | 101 |
| Bub1_292(9) | KMDELHKKL | 102 |
| Bub1_353(9) | TYQQTPVNM | 103 |
| Bub1_414(10) | EFKPQSGAEI | 104 |
| Bub1_474(10) | MFQAPTLPDI | 105 |
| Bub1_552(10) | TFGERSVSRL | 106 |
| Bub1_574(9) | EFLDDSTVW | 107 |
| Bub1_581(10) | VWGIRCNKTL | 108 |
| Bub1_741(10) | PWDDKLIFKL | 109 |
| Bub1_837(9) | LMERLKPSM | 110 |
| Bub1_880(10) | LYKNTPEKVM | 111 |
| Bub1_922(9) | NFILGNGFL | 112 |
| Bub1_952(10) | DMKLFPKGTI | 113 |
| Bub1_995(10) | VYCMLFGTYM | 114 |
| Bub1_1018(10) | LFRRLPHLDM | 115 |
| Bub1_1037(10) | MWNEFFHVML | 116 |
| Bub1_1057(10) | VFQQHYTNKI | 117 |

TABLE 12
Bub1 A2 peptides

| Peptide | Amino Acid Sequence | SEQ ID NO |
|---|---|---|
| Bub1_781(9) | QLGSKLVYV | 118 |
| Bub1_998(9) | MLFGTYMKV | 119 |
| Bub1_1026(9) | DMWNEFFHV | 120 |
| Bub1_392(9) | TVTDSMFAV | 121 |
| Bub1_900(9) | RMLYMIEQV | 122 |
| Bub1_1017(9) | GLFRRLPHL | 123 |
| Bub1_903(9) | YMIEQVHDC | 124 |
| Bub1_750(9) | LLSGLSKPV | 125 |
| Bub1_44(9) | YLITLLEHL | 126 |
| Bub1_88(9) | FLYNHGIGT | 127 |
| Bub1_292(9) | KMDELHKKL | 128 |
| Bub1_785(9) | KLVYVHHLL | 129 |
| Bub1_268(9) | WVNEDRHYM | 130 |
| Bub1_699(9) | RLTDTDAAI | 131 |
| Bub1_943(9) | ALIDLGQSI | 132 |
| Bub1_746(9) | LIFKLLSGL | 133 |
| Bub1_235(9) | VMYCKEKLI | 134 |
| Bub1_68(9) | FITYCLKFA | 135 |
| BUb1_613(9) | KLPVESVHI | 136 |
| Bub1_471(9) | IMNMFQAPT | 137 |
| Bub1_780(10) | FQLGSKLVYV | 138 |
| Bub1_575(10) | FLDDSTVWGI | 139 |
| Bub1_792(10) | LLGEGAFAQV | 140 |
| Bub1_749(10) | KLLSGLSKPV | 141 |
| Bub1_25(10) | GEWERYIQWV | 142 |
| Bub1_954(10) | KLFPKGTIFT | 143 |
| Bub1_997(10) | CMLFGTYMKV | 144 |
| Bub1_745(10) | KLIFKLLSGL | 145 |
| Bub1_686(10) | VLTCEAELGV | 146 |
| Bub1_879(10) | NLYKNTPEKV | 147 |
| Bub1_88(10) | FLYNHGIGTL | 148 |
| Bub1_287(10) | QLLKQKMDEL | 149 |
| Bub1_836(10) | QLMERLKPSM | 150 |
| Bub1_613(10) | KLPVESVHIL | 151 |
| Bub1_722(10) | WMQMSSLGTV | 152 |
| Bub1_470(10) | FIMNMFQAPT | 153 |
| Bub1_141(10) | RLFQTRLTET | 154 |
| Bub1_982(10) | WNYQIDYFGV | 155 |
| Bub1_447(10) | GMVQATPSKV | 156 |
| Bub1_1048(10) | DLLRQKLKKV | 157 |

TABLE 13
C10orf3 A24 peptides

| Peptide | Amino Acid Sequence | SEQ ID NO |
|---|---|---|
| C10orf3_193(10) | VYVKGLLAKI | 158 |
| C10orf3_446(10) | QYPATEHRDL | 159 |
| C10orf3_169(10) | EMEIQLKDAL | 160 |
| C10orf3_355(9) | QMQACTLDF | 161 |
| C10orf3_193(10) | VYVKGLLAKI | 162 |
| C10orf3_446(10) | QYPATEHRDL | 163 |
| C10orf3_169(10) | EMEIQLKDAL | 164 |
| C10orf3_355(9) | QMQACTLDF | 165 |

TABLE 14
C10orf3 A2 peptides

| Peptide | Amino Acid Sequence | SEQ ID NO |
|---|---|---|
| C10orf3_197(9) | GLLAKIFEL | 166 |
| C10orf3_376(9) | QLLVILKEL | 167 |
| C10orf3_99(9) | ALLEQLEET | 168 |
| C10orf3_341(9) | KQQEEQTRV | 169 |
| C10orf3_228(9) | YLQEEKQKC | 170 |
| C10orf3_392(9) | TQLESLKQL | 171 |
| C10orf3_282(10) | LLYSQRRADV | 172 |
| C10orf3_50(10) | KLTDKERHRL | 173 |
| C10orf3_350(10) | ALLEQQMQAC | 174 |
| C10orf3_328(10) | LLSQVQFLYT | 175 |

TABLE 15
HIFPH3 A24 peptides

| Peptide | Amino Acid Sequence | SEQ ID NO |
|---|---|---|
| HIFPH3_6(9) | IMRLDLEKI | 176 |
| HIFPH3_27(9) | GFCYLDNFL | 177 |
| HIFPH3_92(10) | SFLLSLIDRL | 178 |
| HIFPH3_112(10) | YYVKERSKAM | 179 |
| HIFPH3_155(10) | NWDAKLHGGI | 180 |
| HIFPH3_167(9) | IFPEGKSFI | 181 |
| HIFPH3_173(9) | SFIADVEPI | 182 |
| HIFPH3_295(9) | RYAMTVWYF | 183 |

TABLE 16
HIFPH3 A2 peptides

| Peptide | Ammino Acid Sequence | SEQ ID NO |
|---|---|---|
| HIFPH3_93(9) | FLLSLIDRL | 184 |
| HIFPH3_30(9) | YLDNFLGEV | 185 |
| HIFPH3_18(9) | YIVPCLHEV | 186 |
| HIFPH3_159(9) | KLHGGILRI | 187 |
| HIFPH3_60(9) | QLAGPRAGV | 188 |
| HIFPH3_42(9) | CVLERVKQL | 189 |
| HIFPH3_22(10) | CLHEVGFCYL | 190 |
| HIFPH3_34(10) | FLGEVVGDCV | 191 |
| HIFPH3_96(10) | SLIDRLVLYC | 192 |
| HIFPH3_93(10) | FLLSLIDRLV | 193 |
| HIFPH3_151(10) | YLNKNWDAKL | 194 |

### (2) Peptide Synthesis

Among the peptides as mentioned above (SEQ ID NOS: 13 to 201), 26 peptides derived from Lengsin (Table 5), 7 peptides derived from BJ-TSA-9 (Table 7), 20 peptides derived from C20orf42 (Table 9), and 4 peptides derived from C10orf3 (Table 13) were synthesized by Fmoc method

### Example 6

### Evaluation of Binding Affinities of the Peptides Derived from Different Antigens to HLA-A*2402 and HLA-A*0201

The binding affinities to HLA-A*2402 of the peptides synthesized in Example 1 were determined by the method as described in a literature (J. Immunol. 164:2565, 2000). A cell line RMA-S-A*2402 cell, which was obtained by stably introducing a chimera MHC gene composed of HLA-A*2402 and H-2 Kb into a mouse lymphoma cell line RMA-S lacking MHC class 1 molecule, was incubated at 26° C. for 18 hours. RMA-S-A*2402 cells were washed with PBS solution, suspended in culture solution OPTI-MEM (Invitrogen) containing 3 μL/mL human β₂-microglobulin and 100 μL/mL each peptide, and incubated at 26° C. for 3 hours and at 37° C. for 3 hours. The cells were washed with PBS solution and treated with anti-HLA-A24 antibodies at 4° C. for 30 minutes. Furthermore, the cells were washed with PBS solution, and treated with a PE-labeled anti-mouse IgG antibody at 4° C. for 30 minutes. The cells were washed, and suspended in 1 ml of PBS solution containing 1% formalin for fixation. The cells were measured by a device for flow cytometry, FACScan (BD Bioscience), and the binding affinity of the peptide was obtained from the mean fluorescence intensity. The binding affinities of 31 peptides examined are shown in FIGS. 7 to 9.

An EB virus-derived peptide (EBV) and HIV virus-derived peptide (HIV), which had been reported to bind to HLA-A*2402 (J. Immunol. 158:3325, 1997 and J. Immunol. 164:2565, 2000, respectively) and were used as positive controls, showed a strong binding activity. An ovalbumin-derived peptide (SL8), which had been reported to bind to H2-Kb (Eur J Immunol. 21:2891, 1991) and was used as a negative control, showed a weak binding activity. All of the 31 peptides examined were demonstrated to bind to HLA-A*2402 in a highly preferable manner. Accordingly, it was found that those 31 peptides were tumor antigen peptides and the proteins from which those peptides derived, BJ-TSA-9, C20orf42 and C10orf3, were tumor antigen proteins.

Similarly, the binding affinities of 23 peptides derived from Lengsin to HLA-A*2402 are shown in FIGS. 10 and 11. The peptides of SEQ ID NOS: 15, 16, 21, 22, 23, 24, 25, 26, 27, 30, 195, 197, 198, 199, 200 and 201 showed a significant binding affinity to HLA-A*2402. Accordingly, it was found that those peptides were tumor antigen peptides and the protein from which those peptides derived, Lengsin, was a tumor antigen protein.

The binding affinity to HLA-A*0201 can be confirmed by using a human lymphoma cell line T2 in the same way. Also, the binding affinities of the peptides derived from BUB1 and HIFPH3 to HLA can be confirmed similarly.

### Example 7

### CTL Induction by Tumor Antigen-Derived Peptides

The fact that the proteins BUB1, C10orf3, C20orf42, Lengsin, HIFPH3 and BJ-TSA-9 are tumor antigen proteins and partial peptides thereof are tumor antigen peptides can be confirmed by evaluating the activities of CTLs induced by the peptides showing the binding affinity to HLA in Example 6. Induction of CTL by a peptide and evaluation of the activity of the induced CTL can be conducted according to a known assay using human peripheral blood mononuclear cells or model animals for human as described in a literature (J. Immunol. 169:1611, 2002, WO 02/47474, Int J. Cancer: 100, 565-570 (2002)). In the case of an assay using human peripheral blood mononuclear cells, peripheral blood is collected from a cancer patient or a healthy subject after obtaining informed-consent, and mononuclear cells are separated by density gradient centrifugation method and cultured in AIM-V culture solution (Invitrogen). After 24-hour-cultivation, nonadherent cells are collected and cultured in AIM-V containing 100 U/mL IL-2. For preparation of antigen-presenting cells, adherent cells are cultured in AIM-V culture solution containing 1000 U/mL IL-4 and 1000 U/mL GM-CSF for 5 days, for another 1 day after addition of each of different tumor antigen peptides, and for another 1 day after addition of 10 ng/mL TNF and 1000 U/mL IFN-α. Non-adherent cells are cultured together with the antigen-presenting cells pulsed with the peptide. The non-adherent cells containing the peptide-pulsed CTL are received the second and third peptide-stimulations on 7 and 14 days after the first peptide-stimulation by using peptide-pulsed antigen presenting cells. After one week from the third stimulation, the cells are co-cultured with target cells (cells expressing HLA to which the administered peptide binds and also a tumor antigen providing the administered peptide or the administered peptide itself, or pulsed with the corresponding peptide previously) labeled with a radioactive agent such as ⁵¹Cr or a nonradioactive agent, and then lysis of the target cells by the CTL are evaluated from the amount of the radioactive or nonradioactive agent in the conditioned medium to assess the activity of the CTL. The activity of CTL can also be assessed by measuring IFN-γ produced in the conditioned medium as result of response between CTL and the target cells using ELISA or ELISPOT.

When using a model animal for human to assess the activity, a tumor antigen peptide suitably formulated is administered to a transgenic mouse expressing human HLA and after about 1 week, spleen cells or other lymphatic tissues are obtained. The cells obtained are stimulated in vitro with the same peptide as administered and cultured for about 5 days. The resulting cells are regard to correspond to a CTL population and the activity of the CTL can be assessed as described above.

### Example 8

### CTL Induction by the C10orf3-Derived Peptide

As described in Example 7, peripheral blood mononuclear cells (PBMCs) of a HLA-A*2402-positive cancer patient were stimulated with a C10orf3-derived peptide, C10orf3_{—}193(10), to induce a CTL. The CTL activity was determined by using as target cells T2A24 cell (produced by introducing HLA-A*2402 gene into T2 cell (ATCC No. CRL-1992) which does not have the gene) and a cancer cell line Sw480 which was positive for HLA-A*2402 and C10orf3 and the results are shown in FIG. 12. The CTL clone induced showed a peptide-specific cytotoxic activity. In addition, the CTL clone induced killed the HLA-A*2402 and C10orf3-positive cancer cell line but not HLA-negative K562 cell. This result confirms that the peptide C10orf3_{—}193(10) (SEQ ID NOS: 158, 162) is a tumor antigen peptide and also that C10orf3 is a tumor antigen protein.

### Example 9

### CTL Induction by the Lengsin-Derived Peptide

Some of the synthesized peptides being potential to bind to HLA-A*2402 were mixed and used for stimulation of PBMCs of HLA-A*2402-positive cancer patients to induce CTLs, as described in Example 7. After the induction, response to each peptide was detected by measuring IFN-γ by ELISPOT and the results are shown in FIG. 13. The peptides of SEQ ID NOS: 22, 23, 26, 27, 198, 200 and 201 induced peptide-specific CTLs. Those results confirm that the peptides of SEQ ID NOS: 22, 23, 26, 27, 198, 200 and 201 are tumor antigen peptides and also that Lengsin is a tumor antigen protein.

Then, PBMCs of a cancer patient was stimulated with the peptide of SEQ ID NO: 22 to induce a CTL and the CTL-inducing activity was detected as a cytotoxic activity. The result is shown in FIG. 14. The CTL showed a cytotoxic activity specifically against the target cell pulsed with the same peptide used for stimulation.

### Example 10

### CTL Induction by the HIFPH3-Derived Peptide

The peptides of SEQ ID NOS: 177, 178, 179 and 183 were mixed and used for stimulation of PBMCs of a HLA-A*2402-positive cancer patient to induce CTLs, as described in Example 7. After the induction, cytotoxic activities against different renal cancer cell lines were detected and the results are shown in FIG. 15. Lymphocytes stimulated with the peptides showed a cytotoxic activity against HLA-A24⁺ HIFPH3⁺ SMKT R-1 cell but not against HLA-A24⁻ HIFPH3⁺ SMKT R-4 cell, HLA-A24⁺ HIFPH3 Caki-1 cell, HLA-A24⁻ HIFPH3⁻ ACHN cell, and HLA⁻ K562 cell. Those results indicate that HLA-A24-restricted CTLs against HIFPH3 were induced by the peptide stimulation and then confirm that peptides HIFPH3_{—}27(9) (SEQ ID NO: 177), HIFPH3_{—}92(10) (SEQ ID NO: 178), HIFPH3_{—}112(10) (SEQ ID NO: 179) and HIFPH3-295(9) (SEQ ID NO: 183) are tumor antigen peptides and that HIFPH3 is a tumor antigen protein.

### Example 11

### CTL Induction by the C20orf42-Derived Peptide

Twenty peptides synthesized for HLA-A*2402 were divided into 5 groups each containing 4 peptides. PBMCs of a HLA-A*2402-positive cancer patients were plated onto 96-well plates and stimulated with each of the groups of peptides to induce CTLs according to Example 7. When PBMCs in 384 wells were stimulated with each group and the cytotoxic activities against the peptides used for the stimulation were examined, cytotoxic activities specific for the peptides were detected in 8 to 14 wells. This result confirms that the C20orf42-derived peptides are tumor antigen peptides and that C20orf42 is a tumor antigen protein.

### INDUSTRIAL APPLICABILITY

The present invention provides novel tumor antigen proteins and peptides as well as uses thereof in the field of cancer immunity. The tumor antigen proteins and peptides derived therefrom can be used for treating cancer patients expressing the tumor antigen proteins.

### BRIEF DESCRIPTION OF DRAWINGS

FIG. 1 shows the expression level of mRNA of Lengsin gene in major different normal tissues and lung cancer cell lines analyzed by RT-PCR. In the figure, the arrows of Lengsin and G3PDH (positive control) indicate the positions of the bands showing the mRNA level of the genes, respectively. The different normal or cancer tissues are as follows: Heart, Brain, Placenta, Lung, Liver, Skeleton Muscle, Kidney, Pancreas, Spleen, Thymus, Prostate, Testis, Ovary, Small Intestine, Large Intestine, PBMC, Adenocarcinoma, Squamous cell carcinoma, and Small cell carcinoma. The followings are the names of the different lung cancer cell lines: LNY-1, A549, LHK2, 1-87, LK79, Sq-1, LC817 and Lu65.

FIG. 2 shows the expression level of mRNA of BJ-TSA-9 gene in major different normal tissues and lung, oral, and other cancer cell lines analyzed by RT-PCR. In the figure, the arrows of BJ-TSA-9 and G3PDH (positive control) indicate the positions of the bands showing the mRNA level of the genes, respectively. The different normal or cancer tissues are as follows: Heart, Brain, Placenta, Lung, Liver, Skeleton Muscle, Kidney, Pancreas, Spleen, Thymus, Prostate, Testis, Ovary, Small Intestine, Large Intestine, PBMC, Adenocarcinoma, Squamous cell carcinoma, and Small cell carcinoma. The followings are the names of the cancer cell lines originated from different organs: LNY-1, A549, LHK2, 1-87, LK79, Sq-1, LC817, Lu65, OSC19, OSC20, OSC30, OSC40, OSC70, HSC2, HSC3, HSC4, KOSC3, HO-1-N-1, R3, SW450, SSTW, HS776, CHC20, CHC32, C1R, T2, 888mel, and LG2mel.

FIG. 3 shows the expression level of mRNA of C20orf42 gene in major different normal tissues and colon and pancreas cancer cell lines analyzed by RT-PCR. In the figure, the arrows of C20orf42 and G3PDH (positive control) indicate the positions of the bands showing the mRNA level of the genes, respectively. The different normal or cancer tissues are as follows: Heart, Brain, Placenta, Lung, Liver, Skeleton Muscle, Kidney, Pancreas, Spleen, Thymus, Prostate, Testis, Ovary, Small Intestine, colon, leukocyte, colon cancer, and pancreatic cancer. The followings are the names of the different colon and pancreas cancer cell lines: SW480, SW620, colo205, WiDR, CFPAC, PK8, SuSu86 and PUN.

FIG. 4 shows the expression level of mRNA of BUB1 gene in major different normal tissues and oral squamous cell carcinoma, renal cancer, large bowel cancer, gastric cancer, pancreas cancer, liver cancer, lymphoma, and melanoma cell lines analyzed by RT-PCR. In the figure, the arrows of BUB1 and G3PDH (positive control) indicate the positions of the bands showing the mRNA level of the genes, respectively. The followings are the names of the cancer cell lines originated from different organs: OSC19, HSC2, Cakil, R3, SW450, SSTW, HS776T, PUN, CHC20, CHC32, C1R, T2, 888mel, and LG2MEL.

FIG. 5 shows the expression level of mRNA of C10orf3 gene in major different normal tissues and lung cancer cell lines analyzed by RT-PCR. In the figure, the arrows of C10orf3 and G3PDH (positive control) indicate the positions of the bands showing the mRNA level of the genes, respectively. The different normal or cancer tissues are as follows: Heart, Brain, Placenta, Lung, Liver, Skeleton Muscle, Kidney, Pancreas, Spleen, Thymus, Prostate, Testis, Ovary, Small Intestine, Large Intestine, PBMC, Adenocarcinoma, and Squamous cell carcinoma, Small cell carcinoma. The followings are the names of the cancer cell lines originated from different organs: LNY-1, A549, LHK2, 1-87, LK79, Sq-1, LC817, and Lu65.

FIG. 6 shows the expression level of mRNA of HIFPH3 gene in major different normal tissues and renal cancer tissues as well as renal cancer cell lines analyzed by RT-PCR. In the figure, the arrows of HIFPH3 and G3PDH and β-actin (positive controls) indicate the positions of the bands showing the mRNA level of the genes, respectively. The different normal or cancer tissues are as follows: Heart, Brain, Placenta, Lung, Liver, Skeleton Muscle, Kidney, Pancreas, Spleen, Thymus, Prostate, Testis, Ovary, Small Intestine, colon, leukocyte, RCC (renal cell cancer). T and N mean tumor and normal tissues, respectively. The followings are the names of the renal cancer cell lines: SMKTR-1, SMKTR-2, SMKTR-3, SMKTR-4, Caki-1, and ACHN.

FIG. 7 shows the binding affinities to HLA-A*2402 of 8 peptides derived from BJ-TSA-9, a EB-virus-derived peptide (EBV) and a HIV-derived peptide (HIV) (positive controls), and an Ovalbumin-derived peptide (SL8) (negative control). In the figure, the horizontal axis indicates mean fluorescence intensity (MFI, corresponding to the binding affinity), and the vertical axis indicates the names of the peptides. The results obtained without peptide addition are mentioned as (−).

FIG. 8 shows the binding affinities to HLA-A*2402 of 20 peptides derived from C20orf42, a EB-virus-derived peptide (EBV) and a HIV-derived peptide (HIV) (positive controls), and an Ovalbumin-derived peptide (SL8) (negative control). In the figure, the horizontal axis indicates mean fluorescence intensity (MFI, corresponding to the binding affinity), and the vertical axis indicates the names of the peptides. The results obtained without peptide addition are mentioned as (−).

FIG. 9 shows the binding affinities to HLA-A*2402 of 4 peptides derived from C10orf3, a EB-virus-derived peptide (EBV) and a HIV-derived peptide (HIV) (positive controls), and an Ovalbumin-derived peptide (SL8) (negative control). In the figure, the horizontal axis indicates mean fluorescence intensity (MFI, corresponding to the binding affinity), and the vertical axis indicates the names of the peptides. The results obtained without peptide addition are mentioned as (−).

FIG. 10 shows the binding affinities to HLA-A*2402 of 16 peptides derived from Lengsin, a HIV-derived peptide (HIV) (positive control), and an Ovalbumin-derived peptide (SL8) (negative control). In the figure, the horizontal axis indicates mean fluorescence intensity (MFI, corresponding to the binding affinity), and the vertical axis indicates the names of the peptides. The results obtained without peptide addition are mentioned as (−).

FIG. 11 shows the binding affinities to HLA-A*2402 of 7 peptides derived from Lengsin, a EB-virus-derived peptide (EBV) (positive control), and an Ovalbumin-derived peptide (SL8) (negative control). In the figure, the horizontal axis indicates mean fluorescence intensity (MFI, corresponding to the binding affinity), and the vertical axis indicates the names of the peptides. The results obtained without peptide addition are mentioned as (−).

FIG. 12 shows the cytotoxic activity of the CTL induced by stimulating PBMCs of a HLA-A*2402-positive cancer patient with C10orf3_{—}193(10) peptide. In the figure, “peptide2” and “Sw480” indicate the results using as target cells T2-A24 cell pulsed with the peptide and a cancer cell line Sw480 which is positive for HLA-A*2402 and C10orf3, respectively. Similarly, “K562” and “(−)” indicate the results using as target cells HLA-negative K562 cell and T2-A24 cell without peptide addition, respectively.

FIG. 13 shows a response of a CTL induced by stimulating PBMCs of HLA-A*2402-positive cancer patients with a Lengsin-derived peptide which was detected by measuring INF-γ with ELOSPOT.

FIG. 14 shows a cytotoxic activity of a CTL induced by stimulating PBMCs of a cancer patient with Lengsin_{—}142(9) peptide. In the figure, “T2A24+peptide” and “T2A24” indicate the results using as target cells T2-A24 cells pulsed and not pulsed with the peptide, respectively. Similarly, “T2A24+HIV” and “K562” indicate the results using as target cells T2-A24 cell pulsed with a HIV-derived peptide and HLA-negative K562 cell, respectively.

FIG. 15 is a graph showing the result of inducing CTLs by stimulating PBMCs of a HLA-A*2402-positive cancer patient with a mixture of HIFPH3-derived peptides (SEQ ID NOS: 177, 178, 179 and 183). The vertical axis indicates the cytotoxic activity. In the figure, “E:T1Cr %”, “E:T3Cr %” and “E:T10Cr %” mean that the ratios between the peptide-stimulated PBMCs and cancer cells are 1, 3, and 10, respectively.

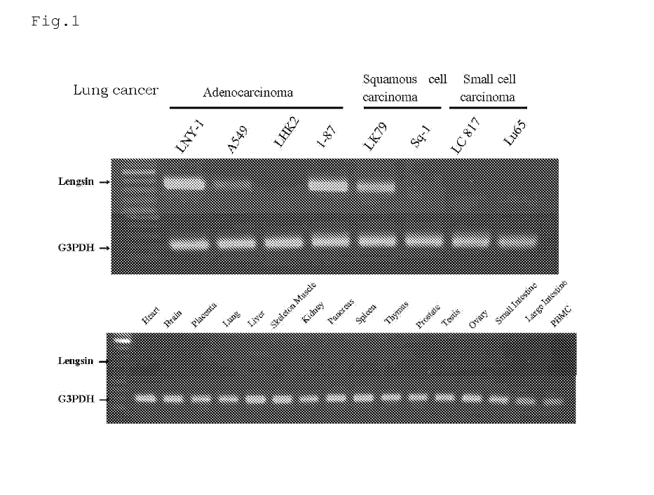

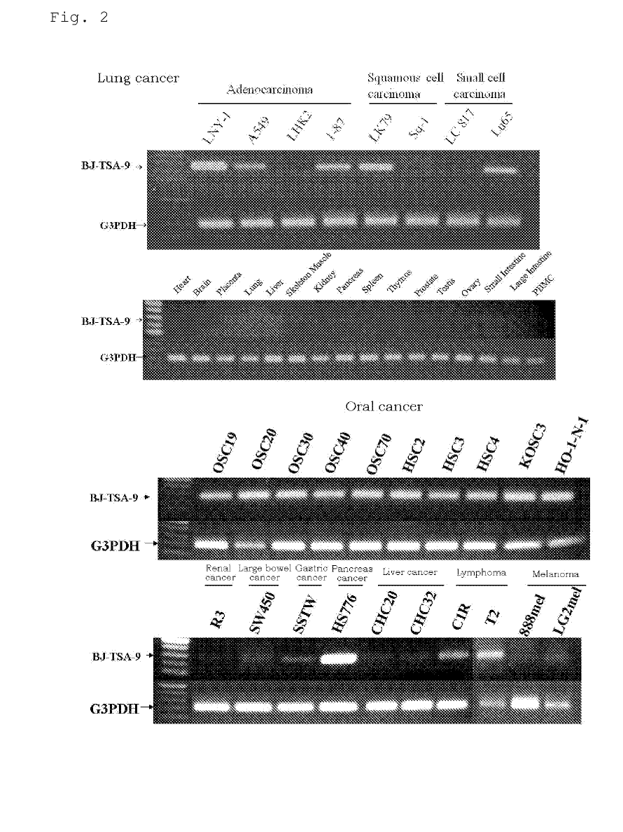

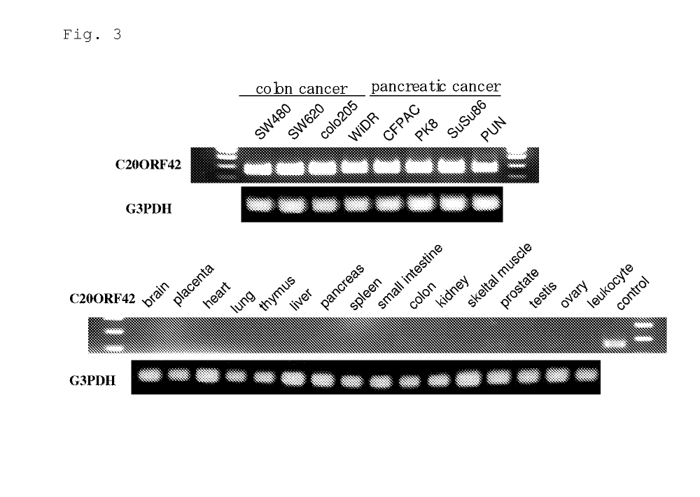

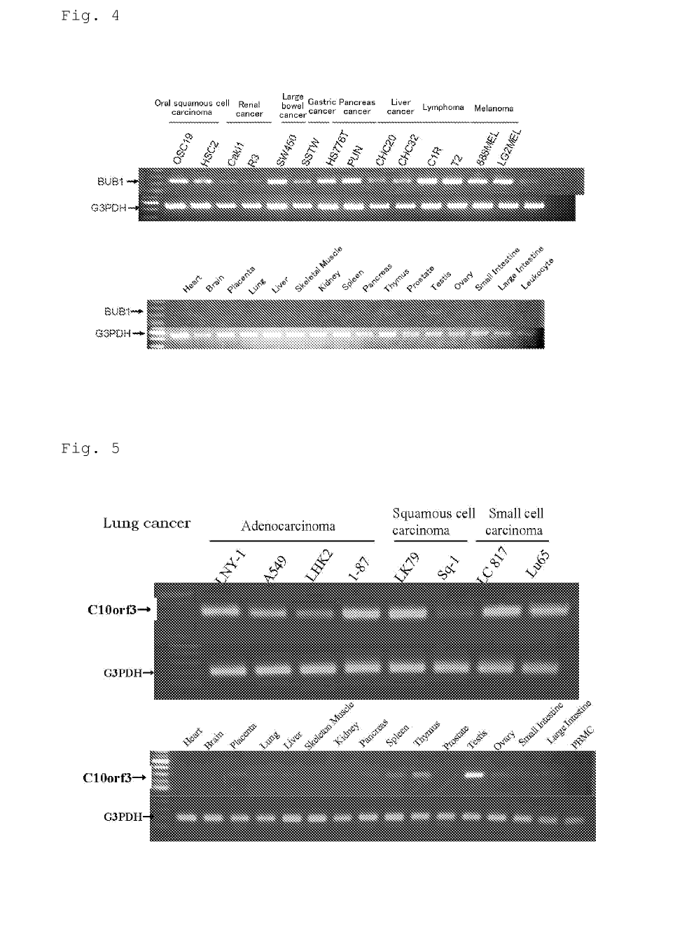

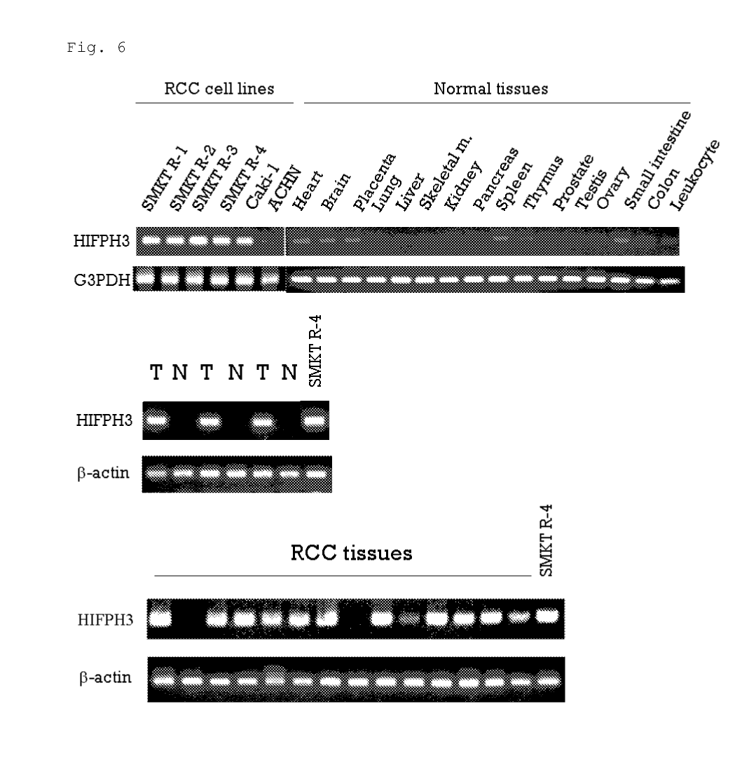

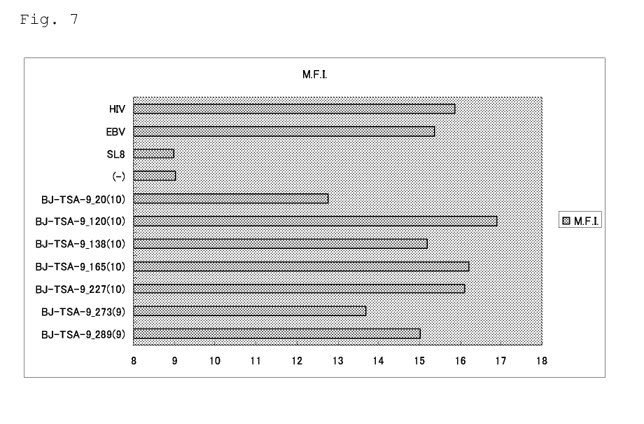

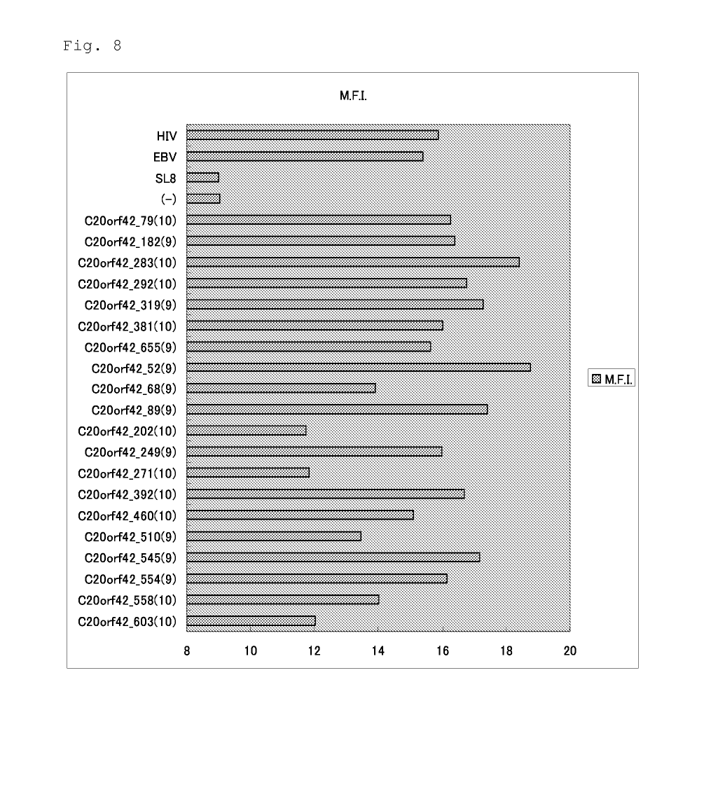

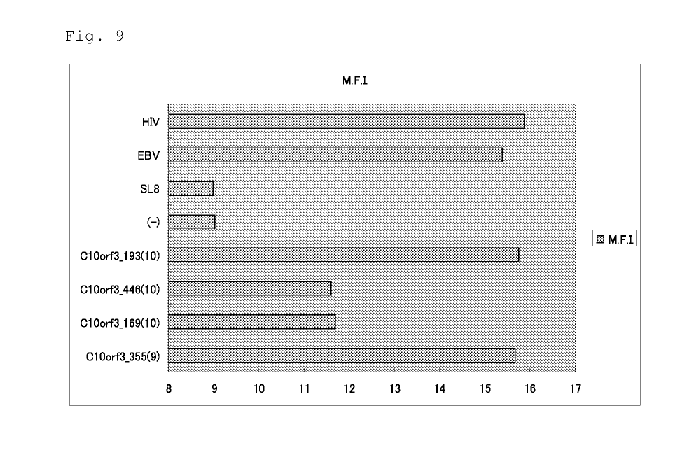

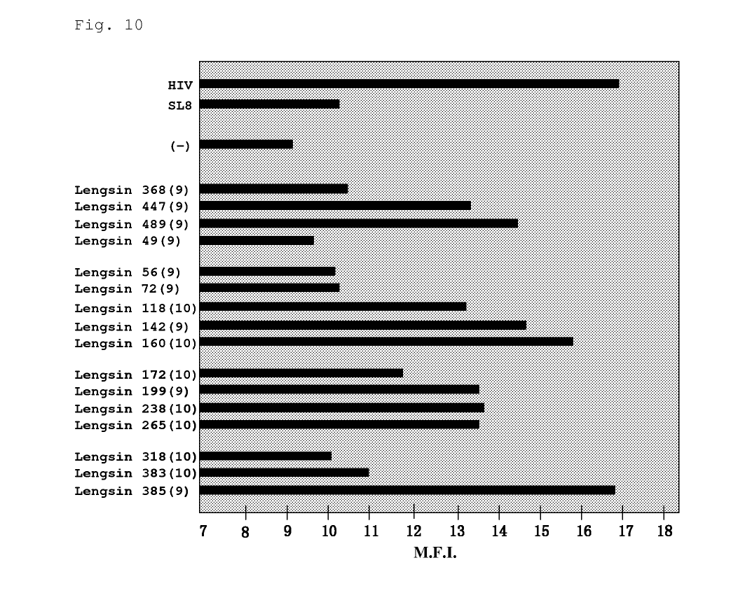

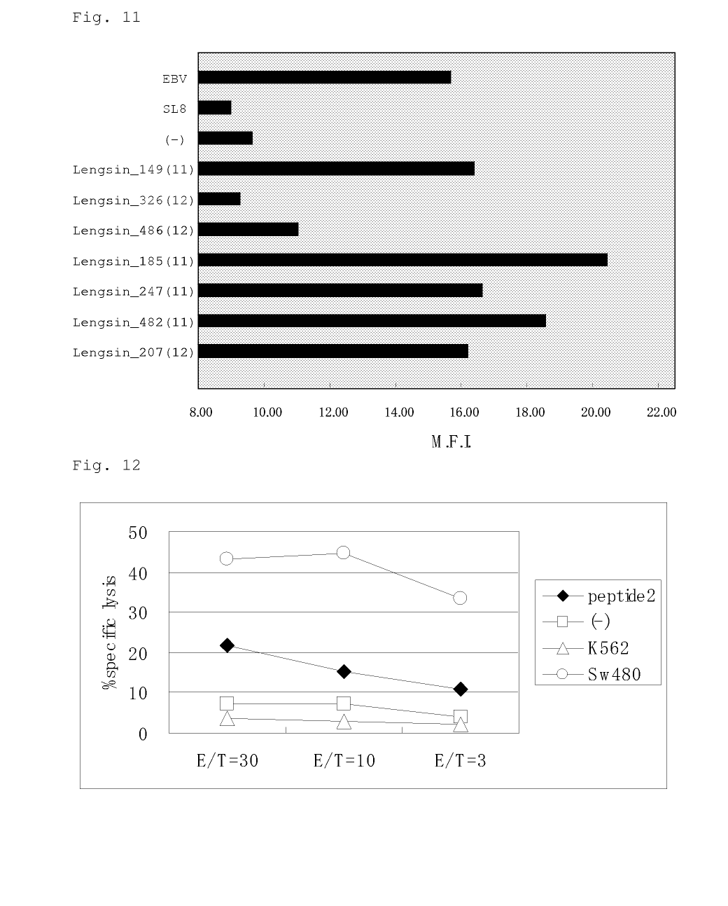

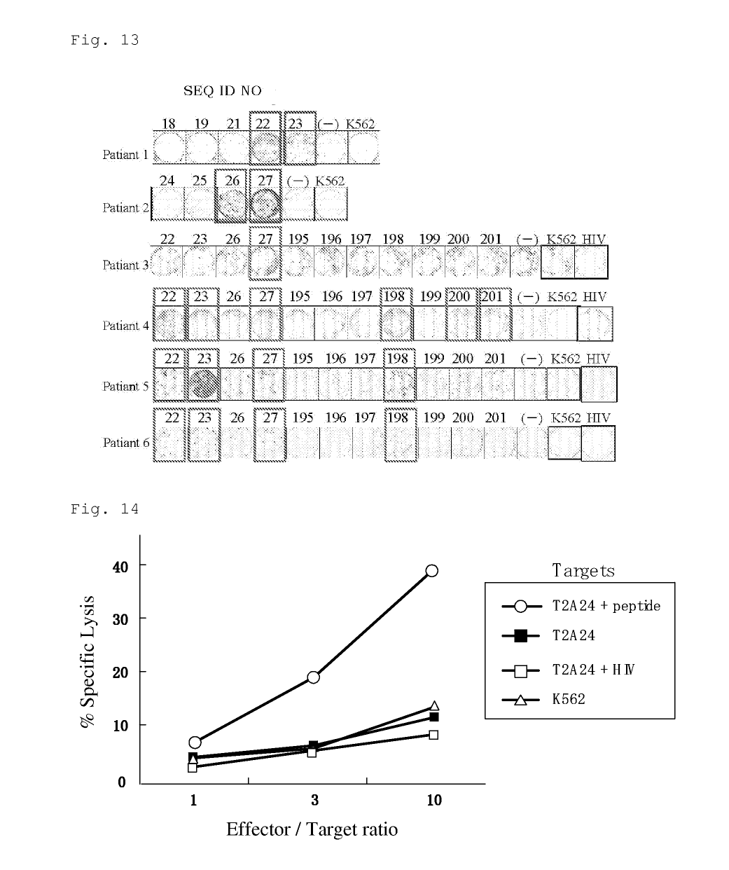

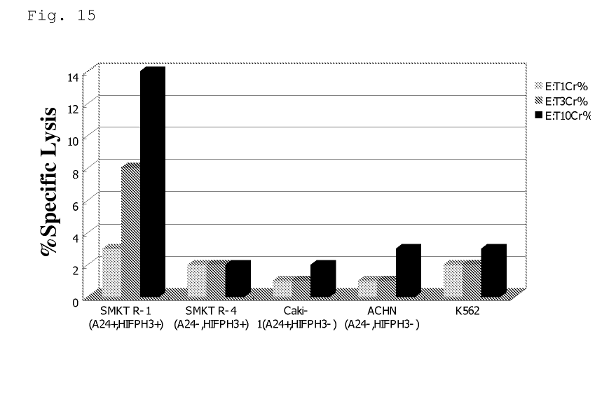

### Sequence Listing Free Text

The amino acid sequences of SEQ ID NOS: 13 to 201 refer to synthetic peptides.

The base sequences of SEQ ID NOS: 202 to 213 refer to primers.

1. A peptide which comprises a partial peptide derived from Lengsin (Glutamate-ammonia ligase (glutamine synthase) domain containing 1, also referred to as GLULD1), BJ-TSA-9 (Hypothetical protein MGC14128), C20orf42 (URP1, also referred to as Kindlerin), BUB1, C10orf3 or HIFPH3 (egl nine homolog3, also referred to as EGLN3) and is capable of binding to an HLA antigen and is recognized by a CTL.

2. The peptide of claim 1, wherein the HLA antigen is HLA-A24 or HLA-A2 antigen.

3. The peptide of claim 2, which comprises the amino acid sequence of any one of SEQ ID NOS: 13 to 201.

4. A peptide which comprises an amino acid sequence which is the same as the amino acid sequence of any one of SEQ ID NOS: 13 to 31, 42 to 49, 59 to 78, 89 to 117, 158 to 165, 176 to 183, and 195 to 201 except that the amino acid at position 2 is substituted by tyrosine, phenylalanine, methionine or tryptophan, and/or the C terminal amino acid by phenylalanine, leucine, isoleucine, tryptophan or methionine, and is capable of binding to HLA-A24 antigen and is recognized by a CTL.

5. A peptide which comprises an amino acid sequence which is the same as the amino acid sequence of any one of SEQ ID NOS:32 to 41, 50 to 58, 79 to 88, 118 to 157, 166 to 175, and 184 to 194 except that the amino acid at position 2 is substituted by leucine, methionine, valine, isoleucine or glutamine and/or the C terminal amino acid by valine or leucine, and is capable of binding to HLA-A2 antigen and is recognized by a CTL.

6. An epitope peptide which comprises the peptide of claim 1.

7. A pharmaceutical composition which comprises the peptide of claim 1 and a pharmaceutically acceptable carrier.

8. A nucleic acid which comprises a polynucleotide encoding the peptide of claim 1.

9. A pharmaceutical composition which comprises the nucleic acid of claim 8 and a pharmaceutically acceptable carrier.

10. A pharmaceutical composition which comprises Lengsin, BJ-TSA-9, C20orf42, BUB1, C10orf3 or HIFPH3 and a pharmaceutically acceptable carrier.

11. The pharmaceutical composition of claim 10, wherein Lengsin comprises the amino acid sequence of SEQ ID NO: 2.

12. The pharmaceutical composition of claim 10, wherein BJ-TSA-9 comprises the amino acid sequence of SEQ ID NO: 4.

13. The pharmaceutical composition of claim 10, wherein C20orf42 comprises the amino acid sequence of SEQ ID NO: 6.

14. The pharmaceutical composition of claim 10, wherein BUB1 comprises the amino acid sequence of SEQ ID NO: 8.

15. The pharmaceutical composition of claim 10, wherein C10orf3 comprises the amino acid sequence of SEQ ID NO: 10.

16. The pharmaceutical composition of claim 10, wherein HIFPH3 comprises the amino acid sequence of SEQ ID NO: 12.

17. A pharmaceutical composition which comprises a nucleic acid comprising a polynucleotide encoding Lengsin, BJ-TSA-9, C20orf42, BUB1, C10orf3 or HIFPH3 and a pharmaceutically acceptable carrier.

18. The pharmaceutical composition of claim 17, wherein the polynucleotide encoding Lengsin comprises the base sequence of SEQ ID NO: 1, or encodes the amino acid sequence of SEQ ID NO: 2.

19. The pharmaceutical composition of claim 17, wherein the polynucleotide encoding BJ-TSA-9 comprises the base sequence of SEQ ID NO: 3, or encodes the amino acid sequence of SEQ ID NO: 4.

20. The pharmaceutical composition of claim 17, wherein the polynucleotide encoding C20orf42 comprises the base sequence of SEQ ID NO: 5, or encodes the amino acid sequence of SEQ ID NO: 6.

21. The pharmaceutical composition of claim 17, wherein the polynucleotide encoding BUB1 comprises the base sequence of SEQ ID NO: 7, or encodes the amino acid sequence of SEQ ID NO: 8.

22. The pharmaceutical composition of claim 17, wherein the polynucleotide encoding C10orf3 comprises the base sequence of SEQ ID NO: 9, or encodes the amino acid sequence of SEQ ID NO: 10.

23. The pharmaceutical composition of claim 17, wherein the polynucleotide encoding HIFPH3 comprises the base sequence of SEQ ID NO: 11, or encodes the amino acid sequence of SEQ ID NO: 12.

24. A method of preparing an antigen presenting cell, wherein a cell having an antigen-presenting ability is brought into contact in vitro with any one of:
(a) the peptide of claim 1,
(b) a nucleic acid comprising a polypeptide encoding the peptide of (a) above,
(c) Lengsin, BJ-TSA-9, C20orf42, BUB1, C10orf3 or HIFPH3, and
(d) a nucleic acid comprising a polynucleotide encoding Lengsin, BJ-TSA-9, C20orf42, BUB1, C10orf3 or HIFPH3.

25. An antigen presenting cell prepared by the method of claim 24.

26. A pharmaceutical composition which comprises the antigen presenting cell of claim 25 and a pharmaceutically acceptable carrier.

27. A method of inducing a CTL, wherein peripheral blood lymphocytes are brought into contact in vitro with any one of:
(a) the peptide of claim 1,
(b) a nucleic acid comprising a polypeptide encoding the peptide of (a) above,
(c) Lengsin, BJ-TSA-9, C20orf42, BUB1, C10orf3 or HIFPH3, and
(d) a nucleic acid comprising a polynucleotide encoding Lengsin, BJ-TSA-9, C20orf42, BUB1, C10orf3 or HIFPH3.

28. A CTL induced by the method of claim 27.

29. A pharmaceutical composition which comprises the CTL of claim 28 and a pharmaceutically acceptable carrier.

30. (canceled)

31. (canceled)

32. An antibody which specifically binds to the peptide of claim 1.

33. An HLA monomer, HLA dimer, HLA tetramer or HLA pentamer which comprises the peptide of claim 1 and an HLA antigen.

34. A reagent for detecting a CTL specific to a tumor antigen peptide derived from Lengsin, BJ-TSA-9, C20orf42, BUB1, C10orf3 or HIFPH3, which comprises as a component the HLA monomer, HLA dimer, HLA tetramer or HLA pentamer of claim 33.

35. A disease marker consisting of a polynucleotide and/or a complementary polynucleotide thereof, wherein the polynucleotide comprises at least 15 contiguous nucleotides from the base sequence of Lengsin, BJ-TSA-9, C20orf42, BUB1, C10orf3 or HIFPH3 gene.

36. The disease marker of claim 35, which is used as a probe or primer for detecting cancer.

37. The disease marker of claim 35, wherein the disease marker derived from Lengsin gene is used for lung adenocarcinoma, lung squamous cell carcinoma or gastric cancer.

38. The disease marker of claim 35, wherein the disease marker derived from BJ-TSA-9 gene is used for leukemia, lung adenocarcinoma, lung squamous cell carcinoma, small cell lung cancer, oral cancer, gastric cancer, pancreas cancer or lymphoma.

39. The disease marker of claim 35, wherein the disease marker derived from C20orf42 gene is used for lung squamous cell carcinoma, lung adenocarcinoma, liver cancer, gastric cancer, leukemia, malignant lymphoma tissues, rectal cancer, colon cancer or pancreas cancer.

40. The disease marker of claim 35, wherein the disease marker derived from BUB1 gene is used for breast cancer, lung adenocarcinoma, lung squamous cell carcinoma, ovarian cancer, oral squamous cell carcinoma, renal cancer, large bowel cancer (colon cancer, rectal cancer), gastric cancer, pancreas cancer, liver cancer, leukemia, lymphoma or melanoma.

41. The disease marker of claim 35, wherein the disease marker derived from C10orf3 gene is used for breast cancer, colon cancer, rectal cancer, renal cancer, gastric cancer, ovarian cancer, liver cancer, pancreas cancer, lung squamous cell carcinoma, lung adenocarcinoma, small cell lung cancer or melanoma.

42. The disease marker of claim 35, wherein the disease marker derived from HIFPH3 gene is used for breast cancer, colon cancer, gastric cancer, renal cancer, pancreas cancer, liver cancer, lung adenocarcinoma or lung squamous cell carcinoma.

43. A method for detecting cancer which comprises the following steps (a), (b) and (c):
(a) allowing RNA prepared from a biological sample of a test subject or complementary polynucleotides transcribed therefrom to hybridize with the disease marker of claim 35;
b) detecting RNA prepared from the biological sample or complementary polynucleotides transcribed therefrom hybridized with the disease marker by using the disease marker as an indicator; and
(c) determining whether or not the test subject has cancer based on the result of the detection in (b).

44. The method of claim 43, wherein the test subject is determined to have cancer in the step (C) when the result of the detection from the test subject is compared with that from a healthy subject and the level of hybridization to the disease marker observed in the test subject is higher than that observed in the healthy subject.

45. A disease marker for cancer which comprises an antibody specifically recognizing Lengsin, BJ-TSA-9, C20orf42, BUB1, C10orf3 or HIFPH3.

46. The disease marker of claim 45, which is used as a probe for detecting cancer.

47. A method for detecting cancer which comprises the following steps (a), (b) and (c):
(a) allowing proteins prepared from a biological sample of a test subject to bind to the disease marker of claim 45;
b) detecting proteins prepared from the biological sample or partial peptides derived therefrom bound to the disease marker by using the disease marker as an indicator; and
(c) determining whether or not the test subject has cancer based on the result of the detection in (b).

48. The method of claim 47, wherein the test subject is determined to have cancer in the step (C) when the result of the detection from the test subject is compared with that from a healthy subject and the level of binding to the disease marker observed in the test subject is higher than that observed in the healthy subject.
